SEQUENCE LISTING (17 sequences)

SEQ ID NO: 1 - protein, 5 aa, Mus sp.
SYDMS

SEQ ID NO: 2 - protein, 7 aa, Mus sp.
KVSSGGG

SEQ ID NO: 3 - protein, 8 aa, Mus sp.
HNYGSFAY

SEQ ID NO: 4 - protein, 11 aa, Mus sp.
QASQSISNHLH

SEQ ID NO: 5 - protein, 11 aa, Mus sp.
YRSQSISNHLH

SEQ ID NO: 6 - protein, 9 aa, Mus sp.
QQSGSWPHT

SEQ ID NO: 7 - DNA, 351 bp, Mus sp.
  CDS (1)..(351)
caggtgcagctggtggagtctgggggaggcgttgtgcagcctggaaggtcccatagactctcctgtgcagcctctggattcaccttcagtagctatgacatgtcttgggttcgccaggctccgggcaagggtctggagtgggtcgcaaaagttagtagtggtggtggtagcaccatcatattagacactgtgcagggccgattcaccatctccagagacaatagtaagaacaccctatacctgcaaatgaactctctgagagccgaggacacagccgtgtattactgtgcaagacataactacggcagttttgcttactggggccaagggactacagtgactgtttctagt

SEQ ID NO: 8 - protein, 117 aa, Mus sp.
QVQLVESGGGVVQPGRSHRLSCAASGFTFSSYDMSWVRQAPGKGLEWVAKVSSGGGSTIILDTVQGRFTISRDNSKNTLYLQMNSLRAEDTAVYYCARHNYGSFAYWGQGTTVTVSS

SEQ ID NO: 9 - DNA, 321 bp, Mus sp.
  CDS (1)..(321)
gagattgtgctaactcagtctccagccaccctgtctctcagcccaggagaaagggcgactctttcctgccaggccagcgaaagtattagcaaccacctacactggtatcaacaaaggcctggtcaagccccaaggcttctcatcaagtatcgttcccagtccatctctgggatccccgccaggttcagtggcagtggatcagggacagatttcaccctcactatctccagtctggagcctgaagattttgcagtctattactgtcaacagagtggcagctggcctcacacgttcggaggggggaccaaggtggaaattaag

SEQ ID NO: 10 - protein, 107 aa, Mus sp.
EIVLTQSPATLSLSPGERATLSCQASESISNHLHWYQQRPGQAPRLLIKYRSQSISGIPARFSGSGSGTDFTLTISSLEPEDFAVYYCQQSGSWPHTFGGGTKVEIK

SEQ ID NO: 11 - protein, 5 aa, Mus sp.
EYYMY

SEQ ID NO: 12 - protein, 17 aa, Mus sp.
RIDPEDGSIDYVEKFKK

SEQ ID NO: 13 - protein, 9 aa, Mus sp.
GKFNYRFAY

SEQ ID NO: 14 - protein, 16 aa, Mus sp.
RSSQSLLHSSGNTLNW

SEQ ID NO: 15 - protein, 7 aa, Mus sp.
LVSKLES

SEQ ID NO: 16 - protein, 9 aa, Mus sp.
MQFTHYPYT

SEQ ID NO: 17 - protein, 347 aa, Homo sapiens
MVAGSDAGRALGVLSVVCLLHCFGFISCFSQQIYGVVYGNVTFHVPSNVPLKEVLWKKQKDKVAELENSEFRAFSSFKNRVYLDTVSGSLTIYNLTSSDEDEYEMESPNITDTMKFFLYVDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

(19) United States
(12) Patent Application Publication
(10) Pub. No.: US 2002/0168360 A1
(43) Pub. Date: Nov. 14, 2002

(54) Methods of preventing or treating inflammatory or autoimmune disorders by administering integrin alphanubeta3 antagonists in combination with other prophylactic or therapeutic agents

(76) Inventors: Christine Dingivan, Germantown, MD (US); Ronald Wilder, Rockville, MD (US)

Correspondence Address: PENNIE AND EDMONDS, 1155 AVENUE OF THE AMERICAS, NEW YORK, NY, 100362711

(21) Appl. No.: 10/091,236

(22) Filed: Mar. 4, 2002

Related U.S. Application Data
(60) Provisional application No. 60/273,098, filed on Mar. 2, 2001.
(60) Provisional application No. 60/316,321, filed on Aug. 31, 2001.
(60) Provisional application No. 60/346,918, filed on Oct. 19, 2001.
(60) Provisional application No. 60/358,424, filed on Feb. 19, 2002.

(51) Int. Cl. A61K 39/395

(52) U.S. Cl. 424/143.1

(57) ABSTRACT

The present invention provides to methods of preventing, treating or ameliorating one or more symptoms associated with an autoimmune or inflammatory disorder utilizing combinatorial therapy. In particular, the present invention provides methods of preventing, treating, or ameliorating one or more symptoms associated with an autoimmune or inflammatory disorder comprising administering to a subject in need thereof one or more integrin α_{V}β₃ antagonists and at least one other prophylactic or therapeutic agent. The present invention also provides compositions and articles of manufacture for use in preventing, treating or ameliorating one or more symptoms associated with an autoimmune or inflammatory disorder.

This application is entitled to and claims priority benefit to U.S. provisional application Serial No. 60/273,098, filed Mar. 2, 2001, U.S. provisional application Serial No. 60/316,321, filed Aug. 31, 2001, U.S. provisional application No. 60/346,918, filed Oct. 19, 2001, and U.S. provisional application Serial No. ______, filed Feb. 19, 2002, each of which is incorporated herein by reference in their entirety.

### 1. INTRODUCTION

The present invention provides to methods of preventing, treating or ameliorating one or more symptoms associated with an autoimmune or inflammatory disorder utilizing combinatorial therapy. In particular, the present invention provides methods of preventing, treating, or ameliorating one or more symptoms associated with an autoimmune or inflammatory disorder comprising administering to a subject in need thereof one or more integrin α_{V}β₃ antagonists and at least one other prophylactic or therapeutic agent. The present invention also provides compositions and articles of manufacture for use in preventing, treating or ameliorating one or more symptoms associated with an autoimmune or inflammatory disorder.

### 2. BACKGROUND OF THE INVENTION

Inflammation is a process by which the body's white blood cells and chemicals protect our bodies from infection by foreign substances, such as bacteria and viruses. It is usually characterized by pain, swelling, warmth and redness of the affected area. Chemicals known as cytokines and prostaglandins control this process, and are released in an ordered and self-limiting cascade into the blood or affected tissues. This release of chemicals increases the blood flow to the area of injury or infection, and may result in the redness and warmth. Some of the chemicals cause a leak of fluid into the tissues, resulting in swelling. This protective process may stimulate nerves and cause pain. These changes, when occurring for a limited period in the relevant area, work to the benefit of the body.

In autoimmune and/or inflammatory disorders, the immune system triggers an inflammatory response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (e.g., the thyroid or pancreas), muscles, joints, and skin. Examples of autoimmune disorders include, but are not limited to, Hashimoto's thyroiditis, pernicious anemia, Addison's disease, type 1 diabetes, rheumatoid arthritis, systemic lupus erythematosus, dermatomyositis, Sjogren's syndrome, dermatomyositis, lupus erythematosus, multiple sclerosis, autoimmune inner ear disease myasthenia gravis, Reiter's syndrome, Graves disease, autoimmune hepatitis, familial adenomatous polyposis and ulcerative colitis.

Rheumatoid arthritis (RA) and juvenile rheumatoid arthritis are types of inflammatory arthritis. Arthritis is a general term that describes inflammation in joints. Some, but not all, types of arthritis are the result of misdirected inflammation. Besides rheumatoid arthritis, other types of arthritis associated with inflammation include the following: psoriatic arthritis, Reiter's syndrome, ankylosing spondylitis arthritis, and gouty arthritis. Rheumatoid arthritis is a type of chronic arthritis that occurs in joints on both sides of the body (such as both hands, wrists or knees). This symmetry helps distinguish rheumatoid arthritis from other types of arthritis. In addition to affecting the joints, rheumatoid arthritis may occasionally affect the skin, eyes, lungs, heart, blood or nerves.

Rheumatoid arthritis affects about 1% of the world's population and is potentially disabling. There are approximately 2.9 million incidences of rheumatoid arthritis in the United States. Two to three times more women are affected than men. The typical age that rheumatoid arthritis occurs is between 25 and 50. Juvenile rheumatoid arthritis affects 71,000 young Americans (aged eighteen and under), affecting six times as many girls as boys.

Rheumatoid arthritis is an autoimmune disorder where the body's immune system improperly identifies the synovial membranes that secrete the lubricating fluid in the joints as foreign. Inflammation results, and the cartilage and tissues in and around the joints are damaged or destroyed. In severe cases, this inflammation extends to other joint tissues and surrounding cartilage, where it may erode or destroy bone and cartilage and lead to joint deformities. The body replaces damaged tissue with scar tissue, causing the normal spaces within the joints to become narrow and the bones to fuse together. Rheumatoid arthritis creates stiffness, swelling, fatigue, anemia, weight loss, fever, and often, crippling pain. Some common symptoms of rheumatoid arthritis include joint stiffness upon awakening that lasts an hour or longer; swelling in a specific finger or wrist joints; swelling in the soft tissue around the joints; and swelling on both sides of the joint. Swelling can occur with or without pain, and can worsen progressively or remain the same for years before progressing. The diagnosis of rheumatoid arthritis is based on a combination of factors, including: the specific location and symmetry of painful joints, the presence of joint stiffness in the morning, the presence of bumps and nodules under the skin (rheumatoid nodules), results of X-ray tests that suggest rheumatoid arthritis, and/or positive results of a blood test called the rheumatoid factor. Many, but not all, people with rheumatoid arthritis have the rheumatoid-factor antibody in their blood. The rheumatoid factor may be present in people who do not have rheumatoid arthritis. Other diseases can also cause the rheumatoid factor to be produced in the blood. That is why the diagnosis of rheumatoid arthritis is based on a combination of several factors and not just the presence of the rheumatoid factor in the blood.

The typical course of the disease is one of persistent but fluctuating joint symptoms, and after about 10 years, 90% of sufferers will show structural damage to bone and cartilage. A small percentage will have a short illness that clears up completely, and another small percentage will have very severe disease with many joint deformities, and occasionally other manifestations of the disease. The inflammatory process causes erosion or destruction of bone and cartilage in the joints. In rheumatoid arthritis, there is an autoimmune cycle of persistent antigen presentation, T-cell stimulation, cytokine secretion, synovial cell activation, and joint destruction. The disease has a major impact on both the individual and society, causing significant pain, impaired finction and disability, as well as costing millions of dollars in healthcare expenses and lost wages. (See, for example, the NIH website and the NIAID website).

Currently available therapy for arthritis focuses on reducing inflammation of the joints with anti-inflammatory or immunosuppressive medications. The first line of treatment of any arthritis is usually anti-inflammatories, such as aspirin, ibuprofen and Cox-2 inhibitors such as celecoxib and rofecoxib. “Second line drugs” include gold, methotrexate and steroids. Although these are well-established treatments for arthritis, very few patients remit on these lines of treatment alone. Recent advances in the understanding of the pathogenesis of rheumatoid arthritis have led to the use of methotrexate in combination with antibodies to cytokines or recombinant soluble receptors. For example, recombinant soluble receptors for tumor necrosis factor (TNF)-α have been used in combination with methotrexate in the treatment of arthritis. However, only about 50% of the patients treated with a combination of methotrexate and anti-TNF-α agents such as recombinant soluble receptors for TNF-α show clinically significant improvement. Many patients remain refractory despite treatment. Difficult treatment issues still remain for patients with rheumatoid arthritis. Many current treatments have a high incidence of side effects or cannot completely prevent disease progression. So far, no treatment is ideal, and there is no cure. Novel therapeutics are needed that more effectively treat rheumatoid arthritis and other autoimmune disorders.

Citation or identification of any reference in Section 2 or any other section of this application shall not be construed as an admission that such reference is available as prior art to the present invention.

### 3. SUMMARY OF THE INVENTION

The present invention is based, in part, on the recognition that integrin α_{V}β₃ antagonists potentiate and synergize with certain anti-inflammatory treatments including, in particular, anti-TNF-α agents and methotrexate. Thus, the invention encompasses treatment protocols that provide better prophylactic and therapeutic profiles than current single agent therapies for autoimmune and/or inflammatory disorders. The invention provides combination therapies for prevention, treatment or amelioration of one or more symptoms associated with an autoimmune or inflammatory disorder in a subject, said combination therapies comprising administering to said subject one or more integrin α_{V}β₃ antagonists and one or more prophylactic or therapeutic agents other than integrin α_{V}β₃ antagonists. In particular, the invention provides combination therapies for prevention, treatment or amelioration of one or more symptoms associated with an autoimmune or inflammatory disorder in a subject, said combination therapies comprising administering to said subject an integrin α_{V}β₃ antagonist, preferably VITAXIN™, and at least one other prophylactic or therapeutic agent which has a different mechanism of action than the integrin α_{V}β₃ antagonist.

The combination of one or more integrin α_{V}β₃ antagonists and one or more prophylactic or therapeutic agents other than integrin α_{V}β₃ antagonists produces a better prophylactic or therapeutic effect in a subject than either treatment alone. In certain embodiments, the combination of an integrin α_{V}β₃ antagonist and a prophylactic or therapeutic agent other than an integrin α_{V}β₃ antagonist achieves a 2 fold, preferably a 3 fold, 4 fold, 5 fold, 6 fold, 7 fold, 8 fold, 9 fold, 10 fold, 15 fold or 20 fold better prophylactic or therapeutic effect in a subject with an autoimmune or inflammatory disorder than either treatment alone. In other embodiments, the combination of an integrin α_{V}β₃ antagonist and a prophylactic or therapeutic agent other than an integrin α_{V}β₃ antagonist achieves a 10%, preferably 15%, 20%, 25%, 30%, 35%, 40%, 45%, 50%, 55%, 60%, 70%, 75%, 80%, 85%, 90%, 95%, 100%, 125%, 150%, or 200% better prophylactic or therapeutic effect in a subject with an autoimmune or inflammatory disorder than either treatment alone. In particular embodiments, the combination of an integrin α_{V}β₃ antagonists and a prophylactic or therapeutic agent other than an integrin α_{V}β₃ antagonist achieves a 20%, preferably a 25%, 30%, 35%, 40%, 45%, 50%, 55%, 60%, 65%, 70%, 75% 85%, 90%, 95% or 98% greater reduction in the inflammation of a particular organ, tissue or joint in a subject with an inflammatory disorder or an autoimmune disorder which is associated with inflammation than either treatment alone. In other embodiments, the combination of one or more integrin α_{V}β₃ antagonists and one or more prophylactic or therapeutic agents other than integrin α_{V}β₃ antagonists has an a more than additive effect or synergistic effect in a subject with an autoimmune or inflammatory disorder.

The combination therapies of the invention enable lower dosages of integrin α_{V}β₃ antagonists and/or less frequent administration of integrin α_{V}β₃ antagonists, preferably VITAXIN™, to a subject with an autoimmune or inflammatory disorder to achieve a prophylactic or therapeutic effect. The combination therapies of the invention enable lower dosages of the prophylactic or therapeutic agents utilized in conjunction with integrin α_{V}β₃ antagonists for the prevention or treatment of an autoimmune or inflammatory disorder and/or less frequent administration of such prophylactic or therapeutic agents to a subject with an autoimmune or inflammatory disorder to achieve a prophylactic or therapeutic effect. The combination therapies of the invention reduce or avoid unwanted or adverse side effects associated with the administration of current single agent therapies and/or existing combination therapies for autoimmune or inflammatory disorders, which in turn improves patient compliance with the treatment protocol.

The prophylactic or therapeutic agents of the combination therapies of the present invention can be administered concomitantly or sequentially to a subject. The prophylactic or therapeutic agents of the combination therapies of the present invention can also be cyclically administered. Cycling therapy involves the administration of a first prophylactic or therapeutic agent for a period of time, followed by the administration of a second prophylactic or therapeutic agent for a period of time and repeating this sequential administration, i.e., the cycle, in order to reduce the development of resistance to one of the agents, to avoid or reduce the side effects of one of the agents, and/or to improve the efficacy of the treatment.

The prophylactic or therapeutic agents of the combination therapies of the invention can be administered to a subject concurrently. The term “concurrently” is not limited to the administration of prophylactic or therapeutic agents at exactly the same time, but rather it is meant that an antagonist of integrin α_{V}β₃ and the other agent are administered to a subject in a sequence and within a time interval such that the integrin α_{V}β₃ antagonist can act together with the other agent to provide an increased benefit than if they were administered otherwise. For example, each prophylactic or therapeutic agent (e.g., VITAXIN™, an anti-TNF-α antibody, or methotrexate) may be administered at the same time or sequentially in any order at different points in time; however, if not administered at the same time, they should be administered sufficiently close in time so as to provide the desired therapeutic or prophylactic effect. Each prophylactic or therapeutic agent can be administered separately, in any appropriate form and by any suitable route. In various embodiments, the prophylactic or therapeutic agents are administered less than 15 minutes, less than 30 minutes, less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 1 0 hours to about 11 hours apart, at about 11 hours to about 12 hours apart, no more than 24 hours apart or no more than 48 hours apart. In preferred embodiments, two or more prophylactic or therapeutic agents are administered within the same patient visit.

The prophylactic or therapeutic agents of the combination therapies can be administered to a subject in the same pharmaceutical composition. Alternatively, the prophylactic or therapeutic agents of the combination therapies can be administered concurrently to a subject in separate pharmaceutical compositions. The prophylactic or therapeutic agents may be administered to a subject by the same or different routes of administration.

The present invention provides methods of preventing, treating, managing or ameliorating an autoimmune or inflammatory disorder or one or more symptoms thereof, said methods comprising administering to a subject in need thereof one or more integrin α_{V}β₃ antagonists and one or more prophylactic or therapeutic agents other than integrin α_{V}β₃ antagonists, which prophylactic or therapeutic agents are currently being used, have been used or are known to be useful in the prevention, treatment or amelioration of one or more symptoms associated with an autoimmune disorder or inflammatory disorder. Examples of integrin α_{V}β₃ antagonists include, but are not limited to, proteins, polypeptides, peptides, fusion proteins, antibodies, antibody fragments, large molecules, or small molecules (less than 10 kD) that blocks inhibit, reduce or neutralize the function, activity and/or expression of integrin α_{V}β₃ In a specific embodiment, the present invention provides a method for preventing, treating, managing or ameliorating an autoimmune or inflammatory disorder or one or more symptoms thereof, said method comprising administering to a subject in need thereof one or more integrin α_{V}β₃ antagonists and one or more prophylactic or therapeutic agents other than integrin α_{V}β₃ antagonists, wherein at least one of the integrin α_{V}β₃ antagonists is an antibody or fragment thereof that immunospecifically binds to integrin α_{V}β₃. In a preferred embodiment, the present invention provides a method for preventing, treating, managing or ameliorating an autoimmune or inflammatory disorder or one or more symptoms thereof, said method comprising administering to a subject in need thereof one or more integrin α_{V}β₃ antagonists and one or more prophylactic or therapeutic agents other than integrin α_{V}β₃ antagonists, wherein at least one of the integrin α_{V}β₃ antagonists is the humanized monoclonal MEDI-522 (known under the trade name VITAXIN™) or an antigen-binding fragment thereof.

Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immunemediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. As described herein in Section 3.1, some autoimmune disorders are associated with an inflammatory condition. Thus, there is overlap between what is considered an autoimmune disorder and an inflammatory disorder. Therefore, some autoimmune disorders may also be characterized as inflammatory disorders. The present invention provides methods of preventing, treating, managing or ameliorating an autoimmune or inflammatory disorder or one or more symptoms thereof, said methods comprising administering to a subject in need thereof one or more integrin α_{V}β₃, antagonists and one or more immunomodulatory agents. Preferably, the immunomodulatory agents are not administered to a subject with an autoimmune or inflammatory disorder whose mean absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³. Thus, in a preferred embodiment, prior to or subsequent to the administration of one or more dosages of one or more immunomodulatory agents to a subject with an autoimmune or inflammatory disorder, the absolute lymphocyte count of said subject is determined by techniques well-known to one of skill in the art, including, e.g., flow cytometry or trypan blue counts.

Examples of immunomodulatory agents include, but are not limited to, methothrexate, leflunomide, cyclophosphamide, cyclosporine A, and macrolide antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators. For clarification regarding T cell receptor modulators and cytokine receptor modulators see Section 3.1. Examples of T cell receptor modulators include, but are not limited to, anti-T cell receptor antibodies (e.g., anti-CD4 monoclonal antibodies, anti-CD3 monoclonal antibodies, anti-CD8 monoclonal antibodies, anti-CD40 ligand monoclonal antibodies, anti-CD2 monoclonal antibodies) and CTLA4-immunoglobulin. Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g., interleukin (IL)-2, IL-3, IL-4, IL-5, IL-6, IL-7, IL-8, IL-9, IL-10, IL-11, IL-12, IL-15, TNF-α, interferon (IFN)-α, IFN-β, IFN-γ, and GM-CSF), anti-cytokine receptor antibodies (e.g., anti-IL-2 receptor antibodies, anti-IL-4 receptor antibodies, anti-IL-6 receptor antibodies, anti-IL-10 receptor antibodies, and anti-IL-12 receptor antibodies), anti-cytokine antibodies (e.g., anti-IFN receptor antibodies, anti-TNF-α antibodies, anti-IL-1β antibodies, anti-IL-6 antibodies, and anti-IL-12 antibodies).

In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents, wherein at least one of the integrin α_{V}β₃ antagonists is an antibody or fragment thereof that immunospecifically binds to integrin α_{V}β₃. In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents, wherein at least one of the integrin α_{V}β₃ antagonists is VITAXIN™ or an antigen-binding fragment thereof. In another preferred embodiment, the present invention provides a method of preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of one or more immunomodulatory agents.

In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of methotrexate or cyclosporin. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™ and a prophylactically or therapeutically effective amount of methotrexate or cyclosporin. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists, a prophylactically or therapeutically effective amount of methotrexate, and a prophylactically or therapeutically effective amount of cyclosporin.

The present invention provides methods for preventing, treating, managing or ameliorating an autoimmune or inflammatory disorder or one or more symptoms thereof, said methods comprising administering to a subject in need thereof one or more integrin α_{V}β₃ antagonists and one or more CD2 antagonists. In particular, the invention provides a method for preventing, treating, managing or ameliorating an autoimmune or inflammatory disorder or one or more symptoms thereof, said method comprising administering to a subject in need thereof a prophylactically or therapeutically effective amount of VITAXIN™ or an antigen-biding fragment thereof and one or more CD2 antagonists.

The present invention provides methods for preventing, treating, managing or ameliorating an autoimmune or inflammatory disorder or one or more symptoms thereof, said methods comprising administering to a subject in need thereof one or more integrin α_{V}β₃ antagonists and one or more CD2 binding molecules (e.g., peptides, polypeptides, proteins, antibodies (MEDI-507), and fusion proteins that immunospecifically bind to a CD2 polypeptide and mediate, directly or indirectly, the depletion of peripheral blood lymphocytes). Preferably, CD2 binding molecules are not administered to a subject with an autoimmune or inflammatory disorder whose absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³. Thus, in a preferred embodiment, prior to or subsequent to the administration of one or more dosages of one or more CD2 binding molecules to a subject with an autoimmune or inflammatory disorder, the mean absolute lymphocyte count of said subject is determined by techniques well-known to one of skill in the art, including, e.g., flow cytometry or trypan blue counts.

In a specific embodiment, the percentage of CD2 polypeptides bound by CD2 binding molecules is assessed after the administration of a first dose of one or more CD2 binding molecules to a subject with an autoimmune or inflammatory disorder and prior to the administration of one or more subsequent doses of one or more CD2 binding molecules. In another embodiment, the percentage of CD2 polypeptides bound by CD2 binding molecules is assessed regularly (e.g., every week, every two weeks, every three weeks, every 4 weeks, every 5 weeks, every 8 weeks, or every 12 weeks) following the administration one or more doses of CD2 binding molecules to a subject with an autoimmune or inflammatory disorder. Preferably, a subject with an autoimmune or inflammatory disorder is administered a subsequent dosage of one or more CD2 binding molecules if the percentage of CD2 polypeptides bound by CD2 binding molecules is less than 80%, preferably less than 75%, less than 70%, less than 65%, less than 50%, less than 45%, less than 40%, less than 35%, less than 30%, less than 25%, or less than 20%. The percentage of CD2 polypeptides bound to CD2 binding molecules can be assessed utilizing techniques well-known to one of skill in the art or described herein.

In a specific embodiment, the present invention provides a method for The present invention provides methods for preventing, treating, managing or ameliorating an autoimmune or inflammatory disorder or one or more symptoms thereof, said methods comprising administering to a subject in need thereof one or more integrin α_{V}β₃ antagonists and a prophylactically or therapeutically effective amount of one or more integrin α_{V}β₃ antagonists and a prophylactically or therapeutically effective amount of one or more CD2 binding molecules. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is an antibody or fragment thereof that immunospecifically binds to integrin α_{V}β₃. In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is VITAXIN™ or an antigen-binding fragment thereof. In another preferred embodiment, the present invention provides a method of preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of one or more CD2 binding molecules.

In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding molecules, wherein at least one of the CD2 binding molecules is soluble LFA-3 polypeptide or LFA3TIP. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents, wherein at least one of the CD2 binding molecules is an antibody or fragment thereof that immunospecifically binds to a CD2 polypeptide. In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents, wherein at least one of CD2 binding molecules is MEDI-507 or an antigen-binding fragment thereof.

In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is an antibody or fragment thereof that immunospecifically binds to integrin α_{V}β₃ and wherein at least one of the CD2 binding molecules is a soluble LFA-3 polypeptide or LFA3TIP.

In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding, wherein at least one of the integrin α_{V}β₃ antagonists is VITAXIN™ or an antigen-binding fragment thereof and wherein at least one of the CD2 binding molecules or antigen-binding fragment thereof. In another preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of one or more CD2 binding, wherein at least one of the CD2 binding molecules or antigen-binding fragment thereof. In yet another preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of MEDI-507 or antigen-binding fragment.

The present invention provides methods of preventing, treating, managing or ameliorating an autoimmune or inflammatory disorder or one or more symptoms thereof, said methods comprising administering to a subject in need thereof one or more integrin α_{V}β₃ antagonists and one or more TNF-α antagonists. Examples of TNF-α antagonists include, but are not limited to, antibodies (e.g., infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379)) soluble TNF-α receptors (e.g., sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192)), IL-10, TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is an antibody or fragment thereof that immunospecifically binds to integrin α_{V}β₃.

In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is VITAXIN™ or an antigen-binding fragment thereof. In another preferred embodiment, the present invention provides a method of preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of one or more TNF-α antagonists.

In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists, wherein at least one of the TNF-α antagonists is a soluble TNF-α receptor such as etanercept (ENBREL™; Immunex) or a fragment, derivative or analog thereof, or an antibody that immunospecifically binds to TNF-α such as infliximab (REMICADE™; Centacor) a derivative, analog or antigen-binding fragment thereof.

In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is an antibody or fragment thereof that immunospecifically binds to integrin α_{V}β₃ and wherein at least one of the TNF-α antagonists is a soluble TNF-α receptor such as etanercept (ENBREL™; imunex) or a fragment, derivative or analog thereof, or an antibody that immunospecifically binds to TNF-α such as infliximab (REMICADE™; Centacor) a derivative, analog or antigen-binding fragment thereof

In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is VITAXIN™ or an antigen-binding fragment thereof and wherein at least one of the TNF-α antagonists is a soluble TNF-α receptor such as etanercept (ENBREL™; Immunex) or a fragment, derivative or analog thereof, or an antibody that immunospecifically binds to TNF-α such as infliximab (REMICADE™; Centacor) a derivative, analog or antigen-binding fragment thereof.

The present invention provides methods of preventing, treating, managing or ameliorating an autoimmune or inflammatory disorder or one or more symptoms thereof, said method comprising administering to a subject in need thereof one or more integrin α_{V}β₃ antagonists and one or more anti-inflammatory agents. Examples of anti-inflammatory agents include, but are not limited to, non-steroidal anti-inflammatory drugs (e.g., aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™)) and steroidal anti-inflammatory drugs (e.g., glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes).

In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more anti-inflammatory agents. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more anti-inflammatory agents, wherein at least one of the integrin α_{V}β₃ antagonists is an antibody or fragment thereof that immunospecifically binds to integrin α_{V}β₃.

In a preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of one or more integrin α_{V}β₃ antagonists and a prophylactically or therapeutically effective amount of one or more anti-inflammatory agents, wherein at least one of the integrin α_{V}β₃ antagonists is VITAXIN™ or an antigen-binding fragment thereof. In another preferred embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™ or an antigen-binding fragment thereof and a prophylactically or therapeutically effective amount of one or more anti-inflammatory agents.

The present invention provides methods of preventing, treating, managing or ameliorating an autoimmune or inflammatory disorder or one or more symptoms thereof, said methods comprising administering to a subject in need thereof one or more integrin α_{V}β₃ antagonists, one or more TNF-α antagonists, and one or more immunomodulatory agents. In a specific embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™, a prophylactically or therapeutically effective amount of a soluble TNF-α receptor (e.g., entanercept), and a prophylactically or therapeutically effective amount of methotrexate. In another embodiment, the present invention provides a method for preventing, treating, managing or ameliorating an autoimmune or inflammatory disorder or one or more symptoms thereof, said method comprising administering to a subject in need thereof a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of methotrexate. The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, and one or more CD2 binding molecules. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept), and a prophylactically or therapeutically effective amount of MEDI-507 or antigen-binding fragment thereof. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of MEDI-507 or antigen-binding fragment thereof.

The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, and one or more anti-inflammatory agents. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept), and a prophylactically or therapeutically effective amount of a steriodal or non-steroidal anti-inflammatory drug. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of a steriodal or non-steroidal anti-inflammatory drug.

The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, one or more immunomodulatory agents, and one or more anti-inflammatory agents. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept) or an antibody that immunospecifically binds to TNF-α (e.g., infliximab or an antigen-binding fragment thereof), a prophylactically or therapeutically effective amount of methotrexate, and a prophylactically or therapeutically effective amount of a steriodal or non-steroidal anti-inflammatory drug. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept) or an antibody that immunospecifically binds to TNF-α (e.g., infliximab or an antigen-binding fragment thereof), a prophylactically or therapeutically effective amount of a CD2 binding molecule (e.g., MEDI-507 or an antigen-binding fragment thereof), and a prophylactically or therapeutically effective amount of a steriodal or non-steroidal anti-inflammatory drug.

The present invention provides methods of preventing, treating, managing or ameliorating one or more symptoms associated with an autoimmune or inflammatory disorder in a subject, said methods comprising administering to said subject one or more integrin α_{V}β₃ antagonists and one or more nucleic acid molecules encoding one or more prophylactic or therapeutic agents other than integrin α_{V}β₃ antagonists. The present invention also provides methods of preventing, treating, managing or ameliorating one or more symptoms associated with an autoimmune or inflammatory disorder in a subject, said methods comprising administering to said subject one or more nucleic acid molecules encoding one or more integrin α_{V}β₃ antagonists and one or more nucleic acid molecules encoding one or more prophylactic or therapeutic agents other than integrin α_{V}β₃ antagonists. The present invention further provides methods of preventing, treating, managing or ameliorating one or more symptoms associated with an autoimmune or inflammatory disorder in a subject, said methods comprising administering to said subject one or more nucleic acid molecules encoding one or more integrin α_{V}β₃ antagonists and one or more nucleic acid molecules encoding one or more prophylactic or therapeutic agents other than integrin α_{V}β₃ antagonists.

The present invention provides pharmaceutical compositions comprising a pharmaceutically acceptable carrier, one or more integrin α_{V}β₃ antagonists, and one or more prophylactic or therapeutic agents other than integrin α_{V}β₃ antagonists. The pharmaceutical compositions of the invention may be used in accordance with the methods of the invention for the prevention, treatment or amelioration of one or more symptoms associated with an autoimmune or inflammatory disorder. Preferably, the pharmaceutical compositions of the invention are sterile and in suitable form for a particular method of administration to a subject with an autoimmune or inflammatory disorder.

In one embodiment, a pharmaceutical composition comprises a pharmaceutically acceptable carrier, one or more integrin α_{V}β₃ antagonists, and one or more immunomodulatory agents. In another embodiment, a pharmaceutical composition comprises a pharmaceutically acceptable carrier, VITAXIN™, and one or more immunomodulatory agents. In another embodiment, a pharmaceutical composition comprises a pharmaceutically acceptable carrier, VITAXIN™, and methotrexate.

In a specific embodiment, a pharmaceutical composition comprises a pharmaceutically acceptable carrier, one or more integrin α_{V}β₃ antagonists, and one or more CD2 binding molecules. In another embodiment, a pharmaceutical composition comprises a pharmaceutically acceptable carrier, VITAXIN™ or an antigen-binding fragment thereof, and one or more CD2 binding molecules. In a preferred embodiment, a pharmaceutical composition comprises a pharmaceutically acceptable carrier, VITAXIN™ or an antigen-binding fragment thereof, and MEDI-507 or an antigen-binding fragment thereof.

In a specific embodiment, a pharmaceutical composition comprises a pharmaceutically acceptable carrier, one or more integrin α_{V}β₃ antagonists, and one or more TNF-α antagonists. In another embodiment, a pharmaceutical composition comprises a pharmaceutically acceptable carrier, VITAXIN™ or an antigen-binding fragment thereof, and one or more TNF-α antagonists. In a preferred embodiment, a pharmaceutical composition comprises a pharmaceutically acceptable carrier, VITAXIN™ or an antigen-binding fragment thereof, and a soluble TNF-α receptor (e.g., etanercept) or an antibody that immunospecifically binds to TNF-α.

In a specific embodiment, a pharmaceutical composition comprises a pharmaceutically acceptable carrier, one or more integrin α_{V}β₃ antagonists, and one or more anti-inflammatory agents. In another embodiment, a pharmaceutical composition comprises a pharmaceutically acceptable carrier, VITAXIN™ or an antigen-binding fragment thereof, and one or more anti-inflammatory agents. In a preferred embodiment, a pharmaceutical composition comprises a pharmaceutically acceptable carrier, VITAXIN™ or an antigen-binding fragment thereof, and a steriodal or non-steriodal anti-inflammatory drug.

In one embodiment, a pharmaceutical composition comprises a pharmaceutically acceptable carrier, one or more integrin α_{V}β₃ antagonists, one or more immunomodulatory agents, and one or more TNF-α antagonists. In another embodiment, a pharmaceutical composition comprises a pharmaceutically acceptable carrier, one or more integrin α_{V}β₃ antagonists, one or more CD2 binding molecules, and one or more TNF-α antagonists. In another embodiment, a pharmaceutical composition comprises a pharmaceutically acceptable carrier, one or more integrin α_{V}β₃ antagonists, one or more anti-inflammatory agents, and one or more TNF-α antagonists. In accordance with these embodiments, preferably, at least one of the integrin α_{V}β₃ antagonists is VITAXIN™ or an antigen-binding fragment thereof.

The compositions and methods described herein are particularly useful for the prevention or treatment of rheumatoid arthritis, spondyloarthropathies (e.g., psoriatic arthritis, ankylosing spondylitis, Reiter's Syndrome (a.k.a., reactive arthritis), inflammatory bowel disease associated arthritis, and undifferentitated spondyloarthropathy), psoriasis, undifferentiated arthropathy, and arthritis. Examples of the types of psoriasis which can be treated in accordance with the compositions and methods of the invention include, but are not limited to, plaque psoriasis, pustular psoriasis, erythrodermic psoriasis, guttate psoriasis and inverse psoriasis. The compositions and methods described herein can also be applied to the prevention, treatment, management or amelioration of one or more symptoms associated with inflammatory osteolysis, other disorders characterized by abnormal bone reabsorption, or disorder characterized by bone loss (e.g., osteoporosis). In a preferred embodiment, the compositions and methods described herein are utilized in prophylactic or therapeutic protocols for the prevention, treatment, management or amelioration of one or more symptoms associated with rheumatoid arthritis. In another preferred embodiment, the compositions and methods described herein are utilized in prophylactic or therapeutic protocols for the prevention, treatment, management or amelioration of one or more symptoms associated with psoriasis or psoriatic arthritis. In another preferred embodiment, the compositions and methods described herein are utilized in prophylactic or therapeutic protocols for the prevention, treatment, management, or amelioration of the symptoms of osteoporosis which are associated with rheumatoid arthritis, psoriatic arthritis or psoriasis, and juvenile chronic arthritis.

The present invention provides article of manufactures comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein said pharmaceutical composition comprises one or more integrin α_{V}β₃ antagonists, one or more prophylactic or therapeutic agents other than integrin α_{V}β₃ antagonists, and a pharmaceutically acceptable carrier. The articles of manufacture of the invention may include instructions regarding the use or administration of a pharmaceutical composition, or other informational material that advises the physician, technician or patient on how to appropriately prevent or treat the disease or disorder in question.

In a specific embodiment, an article of manufacture comprises packaging material and a pharmaceutical composition in suitable form for administration to a subject contained within said packaging material, wherein said pharmaceutical composition comprises an integrin α_{V}β₃ antagonist, an anti-inflammatory agent, and a pharmaceutically acceptable carrier. In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, an immunomodulatory agent, and a pharmaceutically acceptable carrier.

In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, a CD2 binding molecule, and a pharmaceutically acceptable carrier. In a preferred embodiment, an article of manufacture comprises packaging material and a pharmaceutical composition in suitable form for administration to a human, preferably a human with an autoimmune or inflammatory disorder, contained within said packaging material, wherein said pharmaceutical composition comprises VITAXIN™ antagonist, MEDI-507, and a pharmaceutically acceptable carrier.

In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, a TNF-α antagonist, and a pharmaceutically acceptable carrier. In a preferred embodiment, an article of manufacture comprises packaging material and a pharmaceutical composition in suitable form for administration to a human, preferably a human with an autoimmune or inflammatory disorder, contained within said packaging material, wherein said pharmaceutical composition comprises an integrin α_{V}β₃ antagonist, a ENBREL™ or REMICADE™, and a pharmaceutically acceptable carrier.

3.1. Terminology

As used herein, the terms “adjunctive” and “conjunction” are used interchangeably with “in combination” or “combinatorial.”

As used herein, the term “analog” in the context of polypeptides refers to a polypeptide that possesses a similar or identical function as a second polypeptide but does not necessarily comprise a similar or identical amino acid sequence of the second polypeptide, or possess a similar or identical structure of the second polypeptide. A polypeptide that has a similar amino acid sequence refers to a second polypeptide that satisfies at least one of the following: (a) a polypeptide having an amino acid sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the amino acid sequence of a second polypeptide; (b) a polypeptide encoded by a nucleotide sequence that hybridizes under stringent conditions to a nucleotide sequence encoding a second polypeptide of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least 80 contiguous amino acid residues, at least 90 contiguous amino acid residues, at least 100 contiguous amino acid residues, at least 125 contiguous amino acid residues, or at least 150 contiguous amino acid residues; and (c) a polypeptide encoded by a nucleotide sequence that is at least 30%, at least 35%, at least 40%, at least 45%, at least 50%, at least 55%, at least 60%, at least 65%, at least 70%, at least 75%, at least 80%, at least 85%, at least 90%, at least 95% or at least 99% identical to the nucleotide sequence encoding a second polypeptide. A polypeptide with similar structure to a second polypeptide refers to a polypeptide that has a similar secondary, tertiary or quaternary structure to the second polypeptide. The structure of a polypeptide can be determined by methods known to those skilled in the art, including but not limited to, peptide sequencing, X-ray crystallography, nuclear magnetic resonance, circular dichroism, and crystallographic electron microscopy.

To determine the percent identity of two amino acid sequences or of two nucleic acid sequences, the sequences are aligned for optimal comparison purposes (e.g., gaps can be introduced in the sequence of a first amino acid or nucleic acid sequence for optimal alignment with a second amino acid or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position. The percent identity between the two sequences is a function of the number of identical positions shared by the sequences (i.e., % identity=number of identical overlapping positions/total number of positions×100%). In one embodiment, the two sequences are the same length.

The determination of percent identity between two sequences can also be accomplished using a mathematical algorithm. A preferred, non-limiting example of a mathematical algorithm utilized for the comparison of two sequences is the algorithm of Karlin and Altschul, 1990, Proc. Natl. Acad. Sci. U.S.A. 87:2264-2268, modified as in Karlin and Altschul, 1993, Proc. Natl. Acad. Sci. U.S.A. 90:5873-5877. Such an algorithm is incorporated into the NBLAST and XBLAST programs of Altschul et al., 1990, J. Mol. 35 Biol. 215:403. BLAST nucleotide searches can be performed with the NBLAST nucleotide program parameters set, e.g., for score=100, wordlength=12 to obtain nucleotide sequences homologous to a nucleic acid molecules of the present invention. BLAST protein searches can be performed with the XBLAST program parameters set, e.g., to score-50, wordlength=3 to obtain amino acid sequences homologous to a protein molecule of the present invention. To obtain gapped alignments for comparison purposes, Gapped BLAST can be utilized as described in Altschul et al., 1997, Nucleic Acids Res. 25:3389-3402. Alternatively, PSI-BLAST can be used to perform an iterated search which detects distant relationships between molecules (Id.). When utilizing BLAST, Gapped BLAST, and PSI-Blast programs, the default parameters of the respective programs (e.g., of XBLAST and NBLAST) can be used (see, e.g., the NCBI website). Another preferred, non-limiting example of a mathematical algorithm utilized for the comparison of sequences is the algorithm of Myers and Miller, 1988, CABIOS 4:11-17. Such an algorithm is incorporated in the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

The percent identity between two sequences can be determined using techniques similar to those described above, with or without allowing gaps. In calculating percent identity, typically only exact matches are counted.

As used herein, the term “analog” in the context of a non-proteinaceous analog refers to a second organic or inorganic molecule which possess a similar or identical function as a first organic or inorganic molecule and is structurally similar to the first organic or inorganic molecule.

As used herein, the terms “antagonist” and “antagonists” refer to any protein, polypeptide, peptide, antibody, antibody fragment, large molecule, or small molecule (less than 10 kD) that blocks, inhibits, reduces or neutralizes the function, activity and/or expression of another molecule. In various embodiments, an antagonist reduces the function, activity and/or expression of another molecule by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the terms “antibody” and “antibodies” refer to monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies include immunoglobulin molecules and immunologically active fragments of immunoglobulin molecules, i.e., molecules that contain an antigen binding site. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

As used herein, the terms “anti-TNF-α agent”, “TNF-α antagonists” and analogous terms refer to any protein, polypeptide, peptide, fusion protein, antibody, antibody fragment, large molecule, or small molecule that blocks, reduces, inhibits or neutralizes the function, activity and/or expression of tumor necrosis factor alpha (TNF-α). Examples of TNF-α antogonists include, but are not limited to, REMICADE™ and ENBREL™. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

As used herein, the term “CD2 polypeptide” refers to a CD2 glycoprotein (a.k.a. T11 or LFA-2) or fragment thereof. In a preferred embodiment, a CD2 polypeptide is the cell surface 50-55 kDa glycoprotein expressed by immune cells such as T-cells and natural killer (“NK”). The CD2 polypeptide may be from any species. The nucleotide and/or amino acid sequences of CD2 polypeptides can be found in the literature or public databases, or the nucleotide and/or amino acid sequences can be determined using cloning and sequencing techniques known to one of skill in the art. For example, the nucleotide sequence of human CD2 can be found in the GenBank database (see, e.g., Accession Nos. X06143, AH002740, and M16445).

As used herein, the term “cytokine receptor modulator” refers to an agent which modulates the phosphorylation of a cytokine receptor, the activation of a signal transduction pathway associated with a cytokine receptor, and/or the expression of a particular protein such as a cytokine. Such an agent may directly or indirectly modulate the phosphorylation of a cytokine receptor, the activation of a signal transduction pathway associated with a cytokine receptor, and/or the expression of a particular protein such as a cytokine. Thus, examples of cytokine receptor modulators include, but are not limited to, cytokines, fragments of cytokines, fusion proteins and antibodies that immunospecifically binds to a cytokine receptor or a fragment thereof. Further, examples of cytokine receptor modulators include, but are not limited to, peptides, polypeptides (e.g., soluble cytokine receptors), fusion proteins and antibodies that immunospecifically binds to a cytokine or a fragment thereof.

As used herein, the term “dermatological agent” and analogous terms refer to an agent that helps treat skin diseases and complaints. Preferably, a dermatological agent refers to a topical agent used to prevent, treat or ameliorate a skin condition, in particular a skin condition associated with increased T cell infiltration, increased T cell activation, and/or abnormal antigen presentation. In a particularly preferred embodiment, a dermatological agent refers to a topical agent used to prevent, treat or ameliorate psoriasis or one or more symptoms thereof.

As used herein, the term “derivative” in the context of polypeptides refers to a polypeptide that comprises an amino acid sequence which has been altered by the introduction of amino acid residue substitutions, deletions or additions. The term “derivative” as used herein also refers to a polypeptide which has been modified, i. e, by the covalent attachment of any type of molecule to the polypeptide. For example, but not by way of limitation, an antibody may be modified, e.g., by glycosylation, acetylation, pegylation, phosphorylation, amidation, derivatization by known protecting/blocking groups, proteolytic cleavage, linkage to a cellular ligand or other protein, etc. A derivative polypeptide may be produced by chemical modifications using techniques known to those of skill in the art, including, but not limited to specific chemical cleavage, acetylation, formylation, metabolic synthesis of tunicamycin, etc. Further, a derivative polypeptide may contain one or more non-classical amino acids. A polypeptide derivative possesses a similar or identical function as the polypeptide from which it was derived.

As used herein, the term “derivative” in the context of a non-proteinaceous derivative refers to a second organic or inorganic molecule that is formed based upon the structure of a first organic or inorganic molecule. A derivative of an organic molecule includes, but is not limited to, a molecule modified, e.g., by the addition or deletion of a hydroxyl, methyl, ethyl, carboxyl or amine group. An organic molecule may also be esterified, alkylated and/or phosphorylated.

As used herein, the terms “disorder” and “disease” are used interchangeably to refer to a condition in a subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, preferably chronic inflammation. Autoimmune disorders may or may not be associated with inflammation. Moreover, inflammation may or may not be caused by an autoimmune disorder. Thus, certain disorders may be characterized as both autoimmune and inflammatory disorders.

As used herein, the term “epitopes” refers to fragments of a polypeptide or protein having antigenic or immunogenic activity in an animal, preferably in a mammal, and most preferably in a human. An epitope having immunogenic activity is a fragment of a polypeptide or protein that elicits an antibody response in an animal. An epitope having antigenic activity is a fragment of a polypeptide or protein to which an antibody immunospecifically binds as determined by any method well-known to one of skill in the art, for example by immunoassays. Antigenic epitopes need not necessarily be immunogenic.

As used herein, the term “fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of another polypeptide. In a specific embodiment, a fragment of a polypeptide retains at least one function of the polypeptide.

As used herein, the term “functional fragment” refers to a peptide or polypeptide comprising an amino acid sequence of at least 5 contiguous amino acid residues, at least 10 contiguous amino acid residues, at least 15 contiguous amino acid residues, at least 20 contiguous amino acid residues, at least 25 contiguous amino acid residues, at least 40 contiguous amino acid residues, at least 50 contiguous amino acid residues, at least 60 contiguous amino residues, at least 70 contiguous amino acid residues, at least contiguous 80 amino acid residues, at least contiguous 90 amino acid residues, at least contiguous 100 amino acid residues, at least contiguous 125 amino acid residues, at least 150 contiguous amino acid residues, at least contiguous 175 amino acid residues, at least contiguous 200 amino acid residues, or at least contiguous 250 amino acid residues of the amino acid sequence of second, different polypeptide, wherein said peptide or polypeptide retains at least one finction of the second, different polypeptide.

As used herein, the term “fusion protein” refers to a polypeptide that comprises an amino acid sequence of a first protein or functional fragment, analog or derivative thereof, and an amino acid sequence of a heterologous protein (i. e., a second protein or functional fragment, analog or derivative thereof different than the first protein or functional fragment, analog or derivative thereof). In one embodiment, a fusion protein comprises a prophylactic or therapeutic agent fused to a heterologous protein, polypeptide or peptide. In accordance with this embodiment, the heterologous protein, polypeptide or peptide may or may not be a different type of prophylactic or therapeutic agent. For example, two different proteins, polypeptides or peptides with immunomodulatory activity may be fused together to form a fusion protein. In certain embodiments, a fusion protein comprises a protein, polypeptide or peptide with integrin α_{V}β₃ antagonist activity and a heterologous protein, polypeptide, or peptide. In other embodiments, a fusion protein comprises a protein, polypeptide or peptide with immunomodulatory activity and a heterologous protein, polypeptide, or peptide. In other embodiments, a fusion protein comprises a CD2 binding molecule and a heterologous protein, polypeptide, or peptide. In yet other embodiments, a fusion protein comprises a protein, polypeptide or peptide with TNF-α antagonist activity and a heterologous protein, polypeptide, or peptide. In a preferred embodiment, fusion proteins retain or have improved integrin α_{V}β₃ antagonist activity, the immunomodulatory activity or TNF-α antagonist activity relative to the activity of the original protein, polypeptide or peptide prior to being fused to a heterologous protein.

As used herein, the term “host cell” refers to the particular subject cell transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

As used herein, the term “hybridizes under stringent conditions” describes conditions for hybridization and washing under which nucleotide sequences at least 60% (65%, 70%, preferably 75%) identical to each other typically remain hybridized to each other. Such stringent conditions are known to those skilled in the art and can be found in Current Protocols in Molecular Biology, John Wiley & Sons, N.Y. (1989), 6.3.1-6.3.6. In one, non-limiting example stringent hybridization conditions are hybridization at 6×sodium chloride/sodium citrate (SSC) at about 45° C., followed by one or more washes in 0.1×SSC, 0.2% SDS at about 68° C. In a preferred, non-limiting example stringent hybridization conditions are hybridization in 6×SSC at about 45° C., followed by one or more washes in 0.2×SSC, 0.1% SDS at 50-65° C. (i.e., one or more washes at 50° C., 55° C., 60° C. or 65° C.). It is understood that the nucleic acids of the invention do not include nucleic acid molecules that hybridize under these conditions solely to a nucleotide sequence consisting of only A or T nucleotides.

As used herein, the term “immunomodulatory agent” and variations thereof including, but not limited to, immunomodulatory agents, refer to an agent that modulates a host's immune system. In certain embodiments, an immunomodulatory agent is an immunosuppressant agent. In certain other embodiments, an immunomodulatory agent is an immunostimulatory agent. In accordance with the invention, an immunomodulatory agent used in the combination therapies of the invention does not include an integrin α_{V}β₃ antagonist. Immunomodatory agents include, but are not limited to, small molecules, peptides, polypeptides, fusion proteins, antibodies, inorganic molecules, mimetic agents, and organic molecules. In certain embodiments, an immunomodulatory agent used in the combination therapies of the invention is a CD2 binding molecule. In other embodiments, an immunomodulatory agent used in the combination therapies of the invention is not a CD2 binding molecule. In other embodiments, an immunomodulatory agent used in the combination therapies of the invention is a TNF-α antagonist. In other embodiments, an immunomodulatory agent used in the combination therapies of the invention is not a TNF-α antagonist. In other embodiments, an immunomodulatory agent used in the combination therapies of the invention is methotrexate. In yet other embodiments, an immunomodulatory agent used in the combination therapies of the invention is not methotrexate.

As used herein, the term “immunospecifically binds to an antigen” and analogous terms refer to peptides, polypeptides, fusion proteins and antibodies or fragments thereof that specifically bind to an antigen or a fragment and do not specifically bind to other antigens. A peptide or polypeptide that immunospecifically binds to an antigen may bind to other peptides or polypeptides with lower affinity as determined by, e.g., immunoassays, BlAcore, or other assays known in the art. Antibodies or fragments that immunospecifically bind to an antigen may cross-reactive with related antigens. Preferably, antibodies or fragments that immunospecifically bind to an antigen do not cross-react with other antigens. in certain embodiments, the antigen to which a peptide, polypeptide, or antibody immunospecifically binds is a cytokine, a cytokine receptor or a T cell receptor.

As used herein, the term “immunospecifically binds to a CD2 polypeptide” and analogous terms refer to peptides, polypeptides, fusion proteins and antibodies or fragments thereof that specifically bind to a CD2 polypeptide or a fragment thereof and do not specifically bind to other polypeptides. A peptide or polypeptide that immunospecifically binds to a CD2 polypeptide may bind to other peptides or polypeptides with lower affinity as determined by, e.g, immunoassays, BlAcore, or other assays known in the art. Antibodies or fragments that immunospecifically bind to a CD2 polypeptide may be cross-reactive with related antigens. Preferably, antibodies or fragments that immunospecifically bind to a CD2 polypeptide or fragment thereof do not cross-react with other antigens. Antibodies or fragments that immunospecifically bind to a CD2 polypeptide can be identified, for example, by irnmunoassays, BIAcore, or other techniques known to those of skill in the art. An antibody or fragment thereof binds specifically to a CD2 polypeptide when it binds to a CD2 polypeptide with higher affinity than to any cross-reactive antigen as determined using experimental techniques, such as radioimmunoassays (RIA) and enzyme-linked immunosorbent assays (ELISAs). See, e.g., Paul, ed., 1989, Fundamental Immunology Second Edition, Raven Press, New York at pages 332-336 for a discussion regarding antibody specificity.

As used herein, the term “immunospecifically bind to integrin α_{V}β₃” and analogous terms refer to peptides, polypeptides, fusion proteins and antibodies or fragments thereof that specifically bind to an integrin α_{V}β₃ polypeptide or a fragment of an integrin α_{V}β₃ polypeptide and do not specifically bind to other polypeptides. Preferably, antibodies or fragments that immunospecifically bind to an integrin α_{V}β₃ polypeptide or fragment thereof do not cross-react with other antigens. Antibodies or fragments that immunospecifically bind to an integrin α_{V}β₃ polypeptide can be identified, for example, by immunoassays or other techniques known to those of skill in the art. Preferably antibodies or fragments that immunospecifically bind to an integrin α_{V}β₃ polypeptide or fragment thereof only antagonize the activity of integrin α_{V}β₃ and do not significantly antagonize the activity of other integrins.

As used herein, the term “in combination” refers to the use of more than one prophylactic and/or therapeutic agents. The use of the term “in combination” does not restrict the order in which prophylactic and/or therapeutic agents are administered to a subject with an autoimmune or inflammatory disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with an autoimmune or inflammatory disorder.

As used herein, the “integrin α_{V}β₃ antagonist” and analogous terms refer to any protein, polypeptide, peptide, fusion protein, antibody, antibody fragment, large molecule, or small molecule (less than 10 kD) that blocks, inhibits, reduces or neutralizes the function, activity and/or expression of integrin α_{V}β₃. A preferred, non-limiting example of an integrin α_{V}β₃ antagonist is VITAXIN™. In various embodiments, an integrin α_{V}β₃ antagonist reduces the function, activity and/or expression of Integrin α_{V}β₃ by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as PBS.

As used herein, the term “isolated” in the context of a peptide, polypeptide, fusion protein or antibody refers to a peptide, polypeptide, fusion protein or antibody which is substantially free of cellular material or contaminating proteins from the cell or tissue source from which it is derived, or substantially free of chemical precursors or other chemicals when chemically synthesized. The language “substantially free of cellular material” includes preparations of a peptide, polypeptide, fusion protein or antibody in which the peptide, polypeptide, fusion protein or antibody is separated from cellular components of the cells from which it is isolated or recombinantly produced. Thus, a peptide, polypeptide, fusion protein or antibody that is substantially free of cellular material includes preparations of a peptide, polypeptide, fusion protein or antibody having less than about 30%, 20%, 10%, or 5% (by dry weight) of heterologous protein (also referred to herein as a “contaminating protein”). When the peptide, polypeptide, fusion protein or antibody is recombinantly produced, it is also preferably substantially free of culture medium, i. e., culture medium represents less than about 20%, 10%, or 5% of the volume of the protein preparation. When the peptide, polypeptide, fusion protein or antibody is produced by chemical synthesis, it is preferably substantially free of chemical precursors or other chemicals, i.e., it is separated from chemical precursors or other chemicals which are involved in the synthesis of the peptide, polypeptide, fusion protein or antibody. Accordingly such preparations of a peptide, polypeptide, fusion protein or antibody have less than about 30%, 20%, 10%, 5% (by dry weight) of chemical precursors or compounds other than the peptide, polypeptide, fusion protein or antibody of interest. In a preferred embodiment, an integrin α_{V}β₃ antagonist is isolated. In another preferred embodiment, an immunomodulatory agent is isolated. In yet another preferred embodiment, a TNF-α antagonist is isolated.

As used herein, the term “isolated” in the context of nucleic acid molecules refers to a nucleic acid molecule which is separated from other nucleic acid molecules which are present in the natural source of the nucleic acid molecule. Moreover, an “isolated” nucleic acid molecule, such as a cDNA molecule, can be substantially free of other cellular material, or culture medium when produced by recombinant techniques, or substantially free of chemical precursors or other chemicals when chemically synthesized. In a preferred embodiment, a nucleic acid molecule encoding an integrin α_{V}β₃ antagonist is isolated. In another preferred embodiment, a nucleic acid molecule encoding an immunomodulatory agent is isolated. In yet another preferred embodiment, a nucleic acid molecule encoding a TNF-α antagonist is isolated.

As used herein, the phrase “low tolerance” refers to a state in which the patient suffers from side effects from treatment so that the patient does not benefit from and/or will not continue therapy because of the adverse effects.

As used herein, the terms “manage”, “managing” and “management” refer to the beneficial effects that a subject derives from a prophylactic or therapeutic agent, which does not result in a cure of the disease. In certain embodiments, a subject is administered one or more prophylactic or therapeutic agents to “manage” a disorder so as to prevent the progression or worsening of the disorder.

As used herein, the phrase “mild disease” describes arthritic patients with at least 2 swollen joints but not more than 10 tender joints.

As used herein, the terms “non-responsive” and refractory” describe patients treated with a currently available prophylactic or therapeutic agent for an inflammatory disorder or an autoimmune disorder (e.g., methotrexate alone or an anti-TNF-α agent) which is not clinically adequate to relieve one or more symptoms associated with the inflammatory or autoimmune disorder. Typically, such patients suffer from severe, persistently active disease and require additional therapy to ameliorate the symptoms associated with their inflammatory or autoimmune disorder.

As used herein, the terms “nucleic acids” and “nucleotide sequences” include DNA molecules (e.g., cDNA or genomic DNA), RNA molecules (e.g., mRNA), combinations of DNA and RNA molecules or hybrid DNA/RNA molecules, and analogs of DNA or RNA molecules. Such analogs can be generated using, for example, nucleotide analogs, which include, but are not limited to, inosine or tritylated bases. Such analogs can also comprise DNA or RNA molecules comprising modified backbones that lend beneficial attributes to the molecules such as, for example, nuclease resistance or an increased ability to cross cellular membranes. The nucleic acids or nucleotide sequences can be single-stranded, double-stranded, may contain both single-stranded and double-stranded portions, and may contain triple-stranded portions, but preferably is double-stranded DNA.

As used herein, the term “potentiate” refers to an improvement in the efficacy of a prophylactic or therapeutic agent at its common or approved dose.

As used herein, the terms “prophylactic agent” and “prophylactic agents” refer to any agent(s) which can be used in the prevention of an autoimmune or inflammatory disorder. In certain embodiments, the term “prophylactic agent” refers to an integrin α_{V}β₃ antagonist (e.g., VITAXIN™). In certain other embodiments, the term “prophylactic agent” does not refer to an integrin α_{V}β₃ antagonist (e.g., VITAXIN™). Preferably, a prophylactic agent is an agent which is known to be useful to, or has been or is currently being used to the prevent or impede the development, onset or progression of an autoimmune or inflammatory disorder.

As used herein, the terms “prevent”, “preventing” and prevention refer to the prevention of the recurrence or onset of one or more symptoms of an autoimmune or inflammatory disorder in a subject resulting from the administration of a prophylactic or therapeutic agent.

As used herein, the term “prophylactically effective amount” refers to that amount of the prophylactic agent sufficient to result in the prevention of the recurrence or onset of one or more symptoms of a disorder.

As used herein, a “prophylactic protocol” refers to a regimen for dosing and timing the administration of one or more prophylactic agents.

A used herein, a “protocol” includes dosing schedules and dosing regimens. The protocols herein are methods of use and include prophylactic and therapeutic protocols.

As used herein, the phrase “side effects” encompasses unwanted and adverse effects of a prophylactic or therapeutic agent. Adverse effects are always unwanted, but unwanted effects are not necessarily adverse. An adverse effect from a prophylactic or therapeutic agent might be harmful or uncomfortable or risky. Side effects from administration of REMICADE™ include, but are not limited to, risk of serious infection and hypersensitivity reactions. Other side effects range from nonspecific symptoms such as fever or chills, pruritus or urticaria, and cardiopulmonary reactions such as chest pain, hypotension, hytertension or dyspnea, to effects such as myalgia and/or arthralgia, rash, facial, hand or lip edema, dysphagia, sore throat, and headache. Yet other side effects include, but are not limited to, abdominal hernia, splenic infarction, splenomegaly, dizziness, upper motor neuron lesions, lupus erythematosus syndrome, rheumatoid nodules, ceruminosis, abdominal pain, diarrhea, gastric ulcers, intestinal obstruction, intestinal perforation, intestinal stenosis, nausea, pancreatitis, vomiting, back pain, bone fracture, tendon disorder or injury, cardiac failure, myocardial ischema, lymphoma, thrombocytopenia, cellulitis, anxiety, confusion, delirium, depression, somnolence, suicide attempts, anemia, abscess, bacterial infections, and sepsis. Side effects from administration of ENBREL™ include, but are not limited to, risk of serious infection and sepsis, including fatalities. Adverse side effects range from serious infections such as pyelonephritis, bronchitis, septic arthritis, abdominal abscess, cellulitis, osteomyelitis, wound infection, pneumonia, foot abscess, leg ulcer, diarrhea, sinusitis, sepsis, headache, nausea, rhinitis, dizziness, pharyngitis, cough, asthenia, abdominal pain, rash, peripheral edema, respirator disorder, dyspepsia, sinusitis, vomiting, mouth ulcer, alopecia, and pheumonitis to other less frequent adverse effects such as heart failure, myocardial infarction, myocardia ischemia, cerebral ischemia, hyertension, hypotension, cholcystitis, pancreatitis, gastrointestinal hemorrhage, bursitis, depression, dyspnea, deep vein thrombosis, pulmonary embolism, membranous glomerulonephropathy, polymyositis, and thrombophlebitis. The side effects resulting from administration of methotrexate include, but are not limited to, serious toxic reactions, which can be fatal, such as unexpectedly severe bone marrow suppression, gastrointestinal toxicity, hepatotoxicity, fibrosis and cirrhosis after prolonged use, lung diseases, diarrhea and ulcerative stomatitis, malignant lymphomas and occasionally fatal severe skin reactions.

As used herein, the term “small molecules” and analogous terms include, but are not limited to, peptides, peptidomimetics, amino acids, amino acid analogs, polynucleotides, polynucleotide analogs, nucleotides, nucleotide analogs, organic or inorganic compounds (i.e,. including heteroorganic and organometallic compounds) having a molecular weight less than about 10,000 grams per mole, organic or inorganic compounds having a molecular weight less than about 5,000 grams per mole, organic or inorganic compounds having a molecular weight less than about 1,000 grams per mole, organic or inorganic compounds having a molecular weight less than about 500 grams per mole, and salts, esters, and other pharmaceutically acceptable forms of such compounds.

As used herein, the terms “subject” and “patient” are used interchangeably. As used herein, the terms “subject” and “subjects” refer to an animal, preferably a mammal including a non-primate (e.g., a cow, pig, horse, cat, dog, rat, and mouse) and a non-primate (e.g., a monkey such as a cynomolgous monkey and a human), and more preferably a human. In one embodiment, the subject is not an immunocompromised or immunosuppressed mammal, preferably a human (e.g., an HIV patient). In another embodiment, the subject is not a mammal, preferably a human, with a lymphocyte count under approximately 500 cells/mm³. In another embodiment, the subject is a mammal, preferably a human, who is or has previously been treated with one or more TNF-α antagonists. In another embodiment, the subject is a mammal, preferably a human, who is or has previously been treated with one or more TNF-α antagonists and methotrexate. In another embodiment, the subject is a mammal, preferably a human, who is not currently being treated with a TNF-α antagonist or methotrexate. In yet another embodiment, the subject is a mammal, preferably a human, with an inflammatory disorder or an autoimmune disorder that is refractory to treatment with a TNF-α antagonist, a non-steriodal anti-inflammatory agent or methotrexate alone. In a preferred embodiment, the subject is a human. In another embodiment, the subject is a human with rheumatoid arthritis, a spondyloarthropathy (e.g., psoriatic arthritis, ankylosing spondylitis, Reiter's Syndrome (a.k.a., reactive arthritis), inflammatory bowel disease associated arthritis, or undifferentitated spondyloarthropathy), undifferentiated arthropathy or psoriasis. In a preferred embodiment, the subject is a human with rheumatoid arthritis, psoriatic arthritis, or psoraisis.

As used herein, the term “synergistic” refers to a combination of prophylactic or therapeutic agents which is more effective than the additive effects of any two or more single agents. A synergistic effect of a combination of prophylactic or therapeutic agents permits the use of lower dosages of one or more of the agents and/or less frequent administration of said agents to a subject with an autoimmune or inflammatory disorder. The ability to utilize lower dosages of prophylactic or therapeutic agents and/or to administer said agents less frequently reduces the toxicity associated with the administration of said agents to a subjectd without reducing the efficacy of said agents in the prevention or treatment of autoimmune or inflammatory disorders. In addition, a synergistic effect can result in improved efficacy of agents in the prevention or treatment of autoimmune or inflammatory disorders. Finally, synergistic effect of a combination of prophylactic or therapeutic agents may avoid or reduce adverse or unwanted side effects associated with the use of any single therapy.

As used herein, the term “T cell receptor modulator” refers to an agent which modulates the phosphorylation of a T cell receptor, the activation of a signal transduction pathway associated with a T cell receptor, and/or the expression of a particular protein such as a cytokine. Such an agent may directly or indirectly modulate the phosphorylation of a T cell receptor, the activation of a signal transduction pathway associated with a T cell receptor, and/or the expression of a particular protein such as a cytokine. Thus, examples of T cell receptor modulators include, but are not limited to, peptides, polypeptides, fusion proteins and antibodies which immunospecifically bind to a T cell receptor or a fragment thereof Further, examples of T cell receptor modulators include, but are not limited to, peptides, polypeptides (e.g., soluble T cell receptors), fusion proteins and antibodies that immunospecifically binds to a ligand for a T cell receptor or a fragment thereof.

As used herein, the terms “therapeutic agent” and “therapeutic agents” refer to any agent(s) which can be used in the prevention, treatment, management or amelioration of one or more symptoms of an autoimmune or inflammatory disease. In certain embodiments, the term “therapeutic agent” refers to an integrin α_{V}β₃ antagonist (e.g., VITAXIN™). In certain other embodiments, the term “therapeutic agent” refers does not refer to an integrin α_{V}β₃ antagonist (e.g., VITAXIN™). Preferably, a therapeutic agent is an agent which is known to be useful for, or has been or is currently being used for the treatment or amelioration of one or more symptoms associated with an autoimmune or inflammatory disorder.

As used herein, the term “therapeutically effective amount” refers to that amount of the therapeutic agent sufficient to result in amelioration of one or more symptoms of a disorder. With respect to the treatment of psoriasis, a therapeutically effective amount preferably refers to the amount of a therapeutic agent that reduces a human's Psoriasis Area and Severity Index (PASI) score by at least 20%, at least 35%, at least 30%, at least 40%, at least 45%, at least 50%, at least 55%, at least 60%, at least 65%, at least 70%, at least 75%, at least 80%, or at least 85%. Alternatively, with respect to the treatment of psoriasis, a therapeutically effective amount preferably refers to the amount of a therapeutic agent that improves a human's global assessment score by at least 25%, at least 35%, at least 30%, at least 40%, at least 45%, at least 50%, at least 55%, at least 60%, at least 65%, at least 70%, at least 75%, at least 80%, at least 85%, at least 90%, or at least 95%.

As used herein, the term “therapeutic protocol” refers to a regimen for dosing and timing the administration of one or more therapeutic agents.

As used herein, the terms “treat”, “treatment” and “treating” refer to the amelioration of one or more symptoms associated with an autoimmune or inflammatory disorder that results from the administration of one or more prophylactic or therapeutic agents. In certain embodiments, such terms refer to a reduction in the swelling of one or more joints, or a reduction in the pain associated with an autoimmune or inflammatory disorder resulting from the administration of one or more prophylactic or therapeutic agents to a subject with such a disorder. In other embodiments, such terms refer to a reduction in a human's PASI score. In other embodiments, such terms refer to an improvement in a human's global assessment score.

### 4. DESCRIPTION OF THE FIGURES

FIGS. 1A-1B: The nucleotide and deduced amino acid sequence of the variable region of the antibody VITAXIN™. FIG. 1A depicts the nucleotide and deduced amino acid sequence for the VITAXIN™ heavy chain variable region (SEQ ID NO:7 and SEQ ID NO:8, respectively). FIG. 1B depicts the nucleotide and deduced amino acid sequence for the VITAXIN™ light chain variable region (SEQ ID NO:9 and SEQ ID NO:10, respectively).

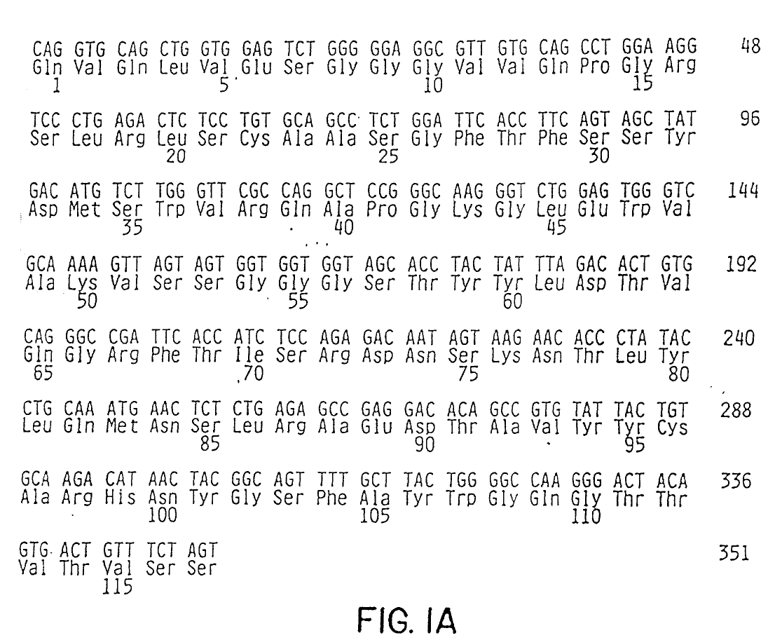

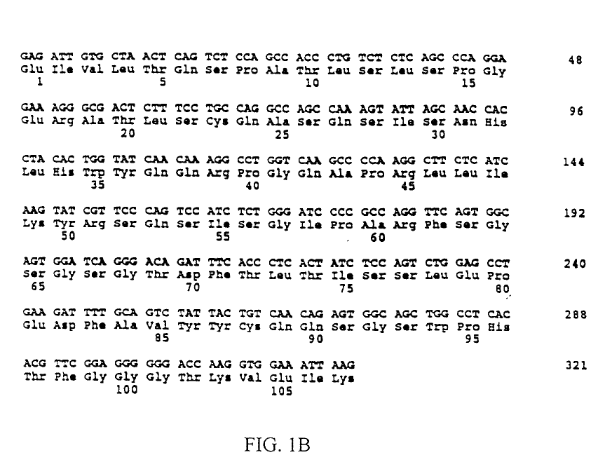

### 5. DETAILED DESCRIPTION OF THE INVENTION

The present invention encompasses treatment protocols that provide better prophylactic and therapeutic profiles than current single agent therapies for autoimmune and/or inflammatory disorders. The invention provides combination therapies for prevention, treatment or amelioration of one or more symptoms associated with an autoimmune or inflammatory disorder in a subject, said combination therapies comprising administering to said subject one or more integrin α_{V}β₃ antagonists and one or more prophylactic or therapeutic agents other than integrin α_{V}β₃ antagonists. In particular, the invention provides combination therapies for prevention, treatment or amelioration of one or more symptoms associated with an autoimmune or inflammatory disorder in a subject, said combination therapies comprising administering to said subject an integrin α_{V}β₃ antagonist, preferably VITAXIN™, and at least one other prophylactic or therapeutic agent which has a different mechanism of action than the integrin α_{V}β₃ antagonist.

The combination of one or more integrin α_{V}β₃ antagonists and one or more prophylactic or therapeutic agents other than integrin α_{V}β₃ antagonists produces a better prophylactic or therapeutic effect in a subject than either treatment alone. In certain embodiments, the combination of an integrin α_{V}β₃ antagonist and a prophylactic or therapeutic agent other than an integrin α_{V}β₃ antagonist achieves a 20%, preferably a 25%, 30%, 35%, 40%, 45%, 50%, 55%, 60%, 65%, 70%, 75%, 80%, 85%, 90%, better prophylactic or therapeutic effect in a subject with an autoimmune or inflammatory disorder than either treatment alone. In particular embodiments, the combination of an integrin α_{V}β₃ antagonists and a prophylactic or therapeutic agent other than an integrin α_{V}β₃ antagonist achieves a 20%, preferably a 25%, 30%, 35%, 40%, 45%, 50%, 55%, 60%, 65% 70%, 75%, 80%, 85%, 90%, 95% or 98% greater reduction in the inflammation of a particular organ, tissue or joint in a subject with an inflammatory disorder or an autoimmune disorder which is associated with inflammation than either treatment alone. In other embodiments, the combination of one or more integrin α_{V}β₃ antagonists and one or more prophylactic or therapeutic agents other than integrin α_{V}β₃ antagonists has an a more than additive effect or synergistic effect in a subject with an autoimmune or inflammatory disorder.

The combination therapies of the invention enable lower dosages of integrin α_{V}β₃ antagonists and/or less frequent administration of integrin α_{V}β₃ antagonists, preferably VITAXIN™, to a subject with an autoimmune or inflammatory disorder to achieve a prophylactic or therapeutic effect. The combination therapies of the invention enable lower dosages of the prophylactic or therapeutic agents utilized in conjunction with integrin α_{V}β₃ antagonists for the prevention or treatment of an autoimmune or inflammatory disorder and/or less frequent administration of such prophylactic or therapeutic agents to a subject with an autoimmune or inflammatory disorder to achieve a prophylactic or therapeutic effect. The combination therapies of the invention reduce or avoid unwanted or adverse side effects associated with the administration of current single agent therapies and/or existing combination therapies for autoimmune or inflammatory disorders, which in turn improves patient compliance with the treatment protocol.

The prophylactic or therapeutic agents of the combination therapies of the present invention can be administered concomitantly, concurrently or sequentially. The prophylactic or therapeutic agents of the combination therapies of the present invention can also be cyclically administered. Cycling therapy involves the administration of a first prophylactic or therapeutic agent for a period of time, followed by the administration of a second prophylactic or therapeutic agent for a period of time and repeating this sequential administration, i.e., the cycle, in order to reduce the development of resistance to one of the agents, to avoid or reduce the side effects of one of the agents, and/or to improve the efficacy of the treatment.

The present invention provides methods of preventing, treating, managing or ameliorating an autoimmune or inflammatorydisorder or one or more symptoms thereof, said methods comprising administering to a subject in need thereof one or more integrin α_{V}β₃ antagonists and one or more prophylactic or therapeutic agents other than integrin α_{V}β₃ antagonists, which prophylactic or therapeutic agents are currently being used, have been used or are known to be useful in the prevention, treatment or amelioration of one or more symptoms associated with an autoimmune disorder or inflammatory disorder. See, e.g., Section 5.2 for non-limiting examples of prophylactic or therapeutic agents that can be administered to a subject in conjunction with one or more integrin α_{V}β₃ antagonists for the prevention, treatment, management or amelioration of one or more symptoms associated with an autoimmune or inflammatory disorder.

The present invention provides methods of preventing, treating, managing or ameliorating an autoimmune or inflammatorydisorder or one or more symptoms thereof, said methods comprising administering to a subject in need thereof one or more integrin α_{V}β₃ antagonists and one or more immunomodulatory agents. Preferably, the immunomodulatory agents are not administered to a subject with an autoimmune or inflammatory disorder whose absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³.

The present invention provides methods for preventing, treating, managing or ameliorating an autoimmune or inflammatorydisorder or one or more symptoms thereof, said methods comprising administering to a subject in need thereof one or more integrin α_{V}β₃ antagonists and one or more CD2 antagonists. In particular, the present invention provides a method for preventing, treating, managing or ameliorating an autoimmune or inflammatorydisorder or one or more symptoms thereof, said methods comprising administering to a subject in need thereof a prophylactically or therapeutically effective amount of one or more VITAXIN™ or an antigen-binding fragment thereof and a prophylactically or therapeutically effective amount of one or more CD2 antagonists.

The present invention also provides methods for preventing, treating, managing or ameliorating an autoimmune or inflammatorydisorder or one or more symptoms thereof, said methods comprising administering to a subject in need thereof one or more integrin α_{V}β₃ antagonists and one or more CD2 binding molecules (e.g., peptides, polypeptides, proteins, antibodies (MEDI-507), and fusion proteins that immunospecifically bind to a CD2 polypeptide and mediate, directly or indirectly, the depletion of peripheral blood lymphocytes). Preferably, CD2 binding molecules are not administered to a subject with an autoimmune or inflammatory disorder whose absolute lymphocyte count is less than 500 cells/mm³, less than 550 cells/mm³, less than 600 cells/MM³, less than 650 cells/mm³, less than 700 cells/mm³, less than 750 cells/mm³, less than 800 cells/mm³, less than 850 cells/mm³ or less than 900 cells/mm³. In particular, the present invention provides methods for preventing, treating, managing or ameliorating an autoimmune or inflammatorydisorder or one or more symptoms thereof, said methods comprising administering to a subject in need thereof a prophylactically or therapeutically effective amount of VITAXIN™ or an antigen-binding fragment thereof and a prophylactically or therapeutically effective amount of MEDI-507 or an antigen-binding fragment thereof.

The present invention provides methods of preventing, treating, managing or ameliorating an autoimmune or inflammatory disorder or one or more symptoms thereof, said methods comprising administering to a subject in need thereof one or more integrin α_{V}β₃ antagonists and one or more anti-angiogenic agents. In particular, the present invention provides methods of preventing, treating, managing or ameliorating an autoimmune or inflammatory disorder or one or more symptoms thereof, said methods comprising administering to a subject in need thereof a prophylactically or therapeutically effective amount of VITAXIN™ or an antigen-binding fragment thereof and a prophylactically or therapeutically effective amount of one or more anti-angiogenic agents.

The present invention provides methods of preventing, treating, managing or ameliorating an autoimmune or inflammatory disorder or one or more symptoms thereof, said methods comprising administering to a subject in need thereof one or more integrin α_{V}β₃ antagonists and one or more TNF-α antagonists. In particular, the present invention provides methods of preventing, treating, managing or ameliorating an autoimmune or inflammatory disorder or one or more symptoms thereof, said methods comprising administering to a subject in need thereof a prophylactically or therapeutically effective amount of VITAXIN™ or an antigen-binding fragment thereof and a prophylactically or therapeutically effective amount of one or more TNF-α antagonists.

The present invention provides methods of preventing, treating, managing or ameliorating an autoimmune or inflammatory disorder or one or more symptoms thereof, said methods comprising administering to a subject in need thereof one or more integrin α_{V}β₃ antagonists and one or more anti-inflammatory agents. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™ or an antigen-binding fragment thereof and a prophylactically or therapeutically effective amount of one or more anti-inflammatory agents.

The present invention provides methods of p preventing, treating, managing or ameliorating an autoimmune or inflammatory disorder or one or more symptoms thereof, said methods comprising administering to a subject in need thereof one or more integrin α_{V}β₃ antagonists, one or more TNF-α antagonists, and one or more immunomodulatory agents. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™, a prophylactically or therapeutically effective amount of one or more TNF-α antagonists, and a prophylactically or therapeutically effective amount of methotrexate or cyclosporin.

The present invention also provides methods of preventing, treating, managing or ameliorating an autoimmune or inflammatory disorder or one or more symptoms thereof, said methods comprising administering to a subject in need thereof one or more integrin α_{V}β₃ antagonists, one or more TNF-α antagonists, and one or more CD2 binding molecules. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™, a prophylactically or therapeutically effective amount of one or more TNF-α antagonists, and a prophylactically or therapeutically effective amount of MEDI-507 or antigen-binding fragment thereof.

The present invention provides methods of preventing, treating, managing or ameliorating an autoimmune or inflammatory disorder or one or more symptoms thereof, said methods comprising administering to a subject in need thereof one or more integrin α_{V}β₃ antagonists, one or more TNF-α antagonists, and one or more anti-inflammatory agents. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™, a prophylactically or therapeutically effective amount of one or more TNF-α antagonists, and a prophylactically or therapeutically effective amount of a steriodal or non-steroidal anti-inflammatory drug.

The present invention provides methods of preventing, treating, managing or ameliorating an autoimmune or inflammatory disorder or one or more symptoms thereof, said methods comprising administering to a subject in need thereof one or more integrin α_{V}β₃ antagonists, one or more TNF-α antagonists, one or more immunomodulatory agents, and one or more anti-inflammatory agents. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™, a prophylactically or therapeutically effective amount of one or more TNF-α antagonists, a prophylactically or therapeutically effective amount of methotrexate, and a prophylactically or therapeutically effective amount of a steriodal or non-steroidal anti-inflammatory drug.

The present invention provides methods for preventing, treating, managing or ameliorating an autoimmune or inflammatory disorder or one or more symptoms thereof, said methods comprising administering to a subject in need thereof a prophylactically or therapeutically effective amount of one or more integrin α_{V}β₃ antagonists, a prophylactically or therapeutically effective amount of one or more TNF-α antagonists, a prophylactically or therapeutically effective amount of one or more CD2 binding molecules, and a prophylactically or therapeutically effective amount of one or more anti-inflammatory agents. In particular, the present invention provides methods for preventing, treating, managing or ameliorating an autoimmune or inflammatory disorder or one or more symptoms thereof, said methods comprising administering to a subject in need thereof a prophylactically or therapeutically effective amount of VITAXIN™ or an antigen-binding fragment thereof, a prophylactically or therapeutically effective amount of one or more TNF-α antagonists, a prophylactically or therapeutically effective amount of MEDI-507 or an antigen-binding fragment thereof, and a prophylactically or therapeutically effective amount of a steriodal or non-steroidal anti-inflammatory drug.

The present invention provides pharmaceutical compositions comprising a pharmaceutically acceptable carrier, one or more integrin α_{V}β₃ antagonists, and one or more prophylactic or therapeutic agents other than integrin α_{V}β₃ antagonists. Any prophylactic or therapeutic agent that are currently being used, have been used or are known to be useful in the prevention, treatment or amelioration of one or more symptoms associated with an autoimmune disorder or inflammatory disorder can be combined with one or more integrin α_{V}β₃ antagonists to form a pharmaceutical composition that is suitable for administration to a subject. Section 5.2 provides non-limiting examples of prophylactic and/or therapeutic agents that can be combined with one or more integrin α_{V}β₃ antagonists to form a pharmaceutical composition that is suitable for administration to a subject. The pharmaceutical compositions of the invention may be used in accordance with the methods of the invention for the prevention, treatment or amelioration of one or more symptoms associated with an autoimmune or inflammatory disorder. Preferably, the pharmaceutical compositions of the invention are sterile and in suitable form for a particular method of administration to a subject with an autoimmune or inflammatory disorder.

The compositions and methods of the invention described herein are useful for the prevention or treatment of autoimmune disorders and/or inflammatory disorders. Examples of autoimmune disorders include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Meniere's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections. The compositions and methods of the invention can be used with one or more conventional therapies that are used to prevent, manage or treat the above diseases.

The compositions and methods described herein are particularly useful for the prevention or treatment of rheumatoid arthritis, spondyloarthropathies (e.g., psoriatic arthritis, ankylosing spondylitis, Reiter's Syndrome (a.k.a., reactive arthritis), inflammatory bowel disease associated arthritis, and undifferentitated spondyloarthropathy), psoriasis, undifferentiated arthropathy, and arthritis. The compositions and methods described herein can also be applied to the prevention, treatment, management or amelioration of one or more symptoms associated with inflammatory osteolysis, other disorders characterized by abnormal bone reabsorption, or disorder characterized by bone loss (e.g., osteoporosis).

The present invention provides article of manufactures comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein said pharmaceutical composition comprises one or more integrin α_{V}β₃ antagonists, one or more prophylactic or therapeutic agents other than integrin α_{V}β₃ antagonists, and a pharmaceutically acceptable carrier. The articles of manufacture of the invention may include instructions regarding the use or administration of a pharmaceutical composition, or other informational material that advises the physician, technician or patient on how to appropriately prevent or treat the disease or disorder in question.

5.1. Integrin UVpf Antagonists

Any integrin α_{V}β₃ antagonist well-known to one of skill in the art may be used in the methods and compositions of the invention. The invention encompasses the use of one or more integrin α_{V}β₃ antagonists in the compositions and methods of the invention. Examples of integrin α_{V}β₃ antagonists include, but are not limited to, proteinaceous agents such as non-catalytic metalloproteinase fragments, RGD peptides, peptide mimetics, fusion proteins, disintegrins or derivatives or analogs thereof, and antibodies that immunospecifically bind to integrin α_{V}β₃ nucleic acid molecules, organic molecules, and inorganic molecules. Non-limiting examples of RGD peptides recognized by integrin α_{V}β₃ include Triflavin. Examples of antibodies that immunospecifically bind to integrin α_{V}β₃ include, but are not limited to, 11D2 (Searle), LM609 (Scripps), and VITAXIN™ (MedImmune, Inc.). Non-limiting examples of small molecule peptidometric integrin α_{V}β₃ antagonists include S836 (Searle) and S448 (Searle). Examples of disintegrins include, but are not limited to, Accutin. The invention also encompasses the use of any of the integrin α_{V}β₃ antagonists disclosed in the following U.S. Patents in the compositions and methods of the invention: U.S. Pat. Nos. 5,149,780; 5,196,511; 5,204,445; 5,262,520; 5,306,620; 5,478,725; 5,498,694; 5,523,209; 5,578,704; 5,589,570; 5,652,109; 5,652,110; 5,693,612; 5,705,481; 5,767,071; 5,770,565; 5,780,426; 5,817,457; 5,830,678; 5,849,692; 5,955,572; 5,985,278; 6,048,861; 6,090,944; 6,096,707; 6,130,231; 6,153,628; 6,160,099; and 6,171,588, each of which is incorporated herein by reference in its entirety.

In certain embodiments, an integrin α_{V}β₃ antagonist is a small organic molecule. In other embodiments, an integrin α_{V}β₃ antagonist is not a small organic molecule. In a preferred embodiment, an integrin α_{V}β₃ antagonist is an antibody that immunospecifically binds to integrin α_{V}β₃. In another preferred embodiment, an integrin α_{V}β₃ antagonist is VITAXIN™, a derivative, analog, or antigen-binding fragment thereof.

In a preferred embodiment, integrin α_{V}β₃ antagonists inhibit or reduce angiogenesis.

In a preferred embodiment, proteins, polypeptides or peptides (including antibodies and fusion proteins) that are utilized as integrin α_{V}β₃ antagonists are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as integrin α_{V}β₃ antagonists are human or humanized.

In accordance with the invention, one or more integrin α_{V}β₃ antagonists are administered to a subject with an inflammatory or autoimmune disorder prior to, subsequent to, or concomitantly with one or more other prophylactic or therapeutic agents which have been used, are currently being used or are known to be useful in the prevention or treatment of said inflammatory or autoimmune disorder.

Nucleic acid molecules encoding proteins, polypeptides, or peptides that function as integrin α_{V}β₃ antagonists, or proteins, polypeptides, or peptides that function as integrin α_{V}β₃ antagonists can be administered to a subject with an inflammatory or autoimmune disorder in accordance with the methods of the invention. Further, nucleic acid molecules encoding derivatives, analogs, fragments or variants of proteins, polypeptides, or peptides that function as integrin α_{V}β₃ antagonists, or derivatives, analogs, fragments or variants of proteins, polypeptides, or peptides that function as integrin α_{V}β₃ antagonists can be administered to a subject with an inflammatory or autoimmune disorder in accordance with the methods of the invention. Preferably, such derivatives, analogs, variants and fragments retain the integrin α_{V}β₃ antagonist activity of the full-length wild-type protein, polypeptide, or peptide.

5.1.1. Antibodies that Immunospecificallv Bind to Integrin α_{V}β₃

It should be recognized that antibodies that immunospecifically bind to integrin α_{V}β₃ and function as antagonists are known in the art. Examples of known antibodies that immunospecifically bind to integrin α_{V}β₃ include, but are not limited to, 11D2 (Searle), LM609 (Scripps), the murine monoclonal LM609 (International Publication No. WO 89/015155, which is incorporated herein by reference in its entirety) and the humanized monoclonal antibody MEDI-522 (a.k.a. VITAXIN™, MedImmune, Inc., Gaithersburg, Md.; Wu et al., 1998, PNAAS USA 95(11):6037-6042; International Publication No. WO 90/33919 and WO 00/78815; and U.S. Pat. No. 5,753,230, each of which is incorporated herein by reference in its entirety).

Antibodies that immunospecifically bind to integrin α_{V}β₃ include, but are not limited to, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies of the present invention include immunoglobulin molecules and immunologically active portions of immunoglobulin molecules, i.e., molecules that contain an antigen binding site that immunospecifically binds to integrin α_{V}β₃. The immunoglobulin molecules of the invention can be of any type (e.g., IgG, IgE, IgM, IgD, IgA and IgY), class (e.g., IgG, IgG₂, IgG₃, IgG₄, IgA₁ and IgA₂) or subclass of imnnunoglobulin molecule. In a preferred embodiment, antibodies that immunospecifically bind to integrin α_{V}β₃ are antagonists of integrin α_{V}β₃. In another preferred embodiment, antibodies that immunospecifically bind to integrin α_{V}β₃ inhibit or reduce angiogenesis.

The antibodies that immunospecifically bind to integrin α_{V}β₃ may be from any animal origin including birds and mammals (e.g., human, murine, donkey, sheep, rabbit, goat, guinea pig, camel, horse, or chicken). Preferably, the antibodies that limmunospecifically bind to integrin α_{V}β₃ are human or humanized monoclonal antibodies. As used herein, “human” antibodies include antibodies having the amino acid sequence of a human immunoglobulin and include antibodies isolated from human immunoglobulin libraries or from mice that express antibodies from human genes.

The antibodies that immunospecifically bind to integrin α_{V}β₃ may be monospecific, bispecific, trispecific or of greater multispecificity. Multispecific antibodies may be specific for different epitopes of integrin α_{V}β₃ or may be specific for both an integrin α_{V}β₃ epitope as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that have a high binding affinity for integrin α_{V}β₃. In a specific embodiment, an antibody that immunospecifically binds to integrin α_{V}β₃ has an association rate constant or kₒₙ rate (antibody (Ab)+antigen (Ag)^{k}^{ₒₙ}→Ab−Ag) of at least 10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s^{31 1}, or at least 10 M⁻¹s⁻¹. In apreferr embodiment, an antibody that immunospecifically binds to integrin α_{V}β₃ has a kₒₙ, of at least 2×10⁵ M⁻¹s⁻¹, at least 5×10⁵ M⁻¹s⁻¹, at least 10⁶ M⁻¹s⁻¹, at least 5×10⁶ M⁻¹s⁻¹, at least 10⁷ M⁻¹s⁻¹, at least 5×10⁷ M⁻¹s⁻¹, or at least 10⁸ M⁻¹s⁻¹.

In another embodiment, an antibody that immunospecifically binds to integrin α_{V}β₃ has a k_{off} rate (antibody (Ab)+antigen (Ag) Ab^{k}^{_{off}}←Ab−Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 10⁻³ s⁻¹, less than 5×10⁻³ s⁻¹, less than 10⁻⁴s⁻¹, less than 5×10⁻⁴ s⁻¹, less than 10⁻⁵s⁻¹, less than 5×10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less thatbn 5×10⁻⁸ s⁻¹, less than 10⁻⁹ s⁻¹, less than 5×10⁻⁹ s⁻¹, or less than 10⁻¹⁰ s^{-1.} In a preferred embodime, an antibody that immunospecifically binds to integrin α_{V}β₃has a kₒₙ, of less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻, less than 5×10⁻¹ s⁻¹, less than 10⁻⁸ s⁻¹, less than 5×10⁻⁸ s⁻¹, less than 10⁻⁹ s⁻¹, less than 5×10⁻⁹ s⁻¹, or less than 10⁻¹⁰ s⁻¹.

In another embodiment, an antibody that immunospecifically binds to integrin α_{V}β₃ has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 10⁵ M⁻¹, at keast 5×10⁵ M⁻¹, at least 10⁶ M⁻¹, at least 5×10⁶ M⁻¹, at least 10⁷ M⁻¹, at least 5×10⁷M⁻¹, at least 10⁸ M¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³ M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody that immunospecifically binds to integrin α_{V}β₃ has a dissociation constant or K_{d}(k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In a specific embodiment, an antibody that immunospecifically binds to integrin α_{V}β₃ is LM609 or an antigen-binding fragment thereof e.g., (one or more complementarity determining regions (CDRs) of LM609). LM609 has the amino acid sequence disclosed, e.g., in International Publication No. WO 89/05155 (which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 as Accession Number HB 9537. In an alternative embodiment, an antibody that immunospecifically binds to integrin α_{V}β₃ is not LM609 or an antigen-binding fragment of LM609.

In a preferred embodiment, an antibody that immunospecifically binds to integrin α_{V}β₃ is VITAXIN™ or an antibody-binding fragment thereof (e.g., one or more CDRs of VITAXIN™). VITAXIN™ is disclosed, e.g., in International Publication No. WO 98/33919 and WO 00/78815, U.S. application Ser. No. 09/339,922, and U.S. Pat. No. 5,753,230, each of which is incorporated herein by reference in its entirety. In an alternative embodiment, an antibody that immunospecifically binds to integrin α_{V}β₃ is not VITAXIN™ or an antigen-binding fragment of VITAXIN™.

The present invention also provides antibodies that immunospecifically bind integrin α_{V}β₃, said antibodies comprising a variable heavy (“VH”) domain having an amino acid sequence of the VH domain for LM609 or VITAXIN™. The present invention also provides antibodies that immunospecifically bind to integrin α_{V}β₃, said antibodies comprising a VH CDR having an amino acid sequence of any one of the VH CDRs listed in Table 1.

TABLE 1
CDR Sequences Of LM609

| CDR | Sequence | SEQ ID NO: |
|---|---|---|
| VH1 | SYDMS | 1 |
| VH2 | KVSSGGG | 2 |
| VH3 | HNYGSFAY | 3 |
| VL1 | QASQSISNHLH | 4 |
| VL2 | YRSQSIS | 5 |
| VL3 | QQSGSWPHT | 6 |

In one embodiment, antibodies that immunospecifically bind to integrin α_{V}β₃ comprise a VH CDR1 having the amino acid sequence of SEQ ID NO: 1. In another embodiment, antibodies that immunospecifically bind to integrin α_{V}β₃ comprise a VH CDR2 having the amino acid sequence of SEQ ID NO:2. In another embodiment, antibodies that immunospecifically bind to integrin α_{V}β₃ comprise a VH CDR3 having the amino acid sequence of SEQ ID NO:3. In a preferred embodiment, antibodies that immunospecifically bind to integrin α_{V}β₃, comprise a VH CDR1 having the amino acid sequence of SEQ ID NO:1, a VH CDR2 having the amino acid sequence of SEQ ID NO:2, and a VH CDR3 having the amino acid sequence of SEQ ID NO:3.

The present invention also provides antibodies that immunospecifically bind to integrin α_{V}β₃, said antibodies comprising a variable light (“VL”) domain having an amino acid sequence of the VL domain for LM609 or VITAXIN™. The present invention also provides antibodies that immunospecifically bind to integrin α_{V}β₃ said antibodies comprising a VL CDR having an amino acid sequence of any one of the VL CDRs listed in Table 1.

In one embodiment, antibodies that immunospecifically bind to integrin α_{V}β₃ comprise a VL CDR1 having the amino acid sequence of SEQ ID NO:4. In another embodiment, antibodies that immunospecifically bind to integrin α_{V}β₃ comprise a VL CDR2 having the amino acid sequence of SEQ ID NO:5. In another embodiment, antibodies that immunospecifically bind to integrin α_{V}β₃ comprise a VL CDR3 having the amino acid sequence of SEQ ID NO:6. In a preferred embodiment, antibodies that immunospecifically bind to integrin α_{V}β₃ comprise a VL CDR1 having the amino acid sequence of SEQ ID NO:4, a VL CDR2 having the amino acid sequence of SEQ ID NO:5, and a VL CDR3 having the amino acid sequence of SEQ ID NO:6.

The present invention also provides antibodies that immunospecifically bind to integrin α_{V}β₃, said antibodies comprising a VH domain disclosed herein combined with a VL domain disclosed herein, or other VL domain. The present invention further provides antibodies that immunospecifically bind to integrin α_{V}β₃, said antibodies comprising a VL domain disclosed herein combined with a VH domain disclosed herein, or other VH domain.

The present invention also provides antibodies that immunospecifically bind to integrin α_{V}β₃, said antibodies comprising one or more VH CDRs and one or more VL CDRs listed in Table 1. In particular, the invention provides for an antibody that immunospecifically binds to integrin α_{V}β₃ said antibody comprising a VH CDR1 and a VL CDR1, a VH CDR1 and a VL CDR2, a VH CDR1 and a VL CDR3, a VH CDR2 and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof of the VH CDRs and VL CDRs listed in Table 1.

In one embodiment, an antibody that immunospecifically binds to integrin α_{V}β₃ comprises a VH CDR1 having the amino acid sequence of SEQ ID NO: 1 and a VL CDR1 having the amino acid sequence of SEQ ID NO:4. In another embodiment, an antibody that immunospecifically binds to integrin α_{V}β₃ comprises a VH CDR1 having the amino acid sequence of SEQ ID NO:1 and a VL CDR2 having the amino acid sequence of SEQ ID NO:5. In another embodiment, an antibody that immunospecifically binds to integrin α_{V}β₃ comprises a VH CDR1 having the amino acid sequence of SEQ ID NO:1 and a VL CDR3 having the amino acid sequence of SEQ ID NO:6.

In another embodiment, an antibody that immunospecifically binds to integrin α_{V}β₃ comprises a VH CDR2 having the amino acid sequence of SEQ ID NO:2 and a VL CDR1 having the amino acid sequence of SEQ ID NO:4. In another embodiment, an antibody that immunospecifically binds to integrin α_{V}β₃ comprises a VH CDR2 having the amino acid sequence of SEQ ID NO:2 and a VL CDR2 having the amino acid sequence of SEQ ID NO:5. In another embodiment, an antibody that immunospecifically binds to integrin α_{V}β₃ comprises a VH CDR2 having the amino acid sequence of SEQ ID NO:2 and a VL CDR3 having the amino acid sequence of SEQ ID NO:6.

In another embodiment, an antibody that immunospecifically binds to integrin α_{V}β₃ comprises a VH CDR3 having the amino acid sequence of SEQ ID NO:3 and a VL CDR1 having the amino acid sequence of SEQ ID NO:4. In another embodiment, an antibody that immunospecifically binds to integrin α_{V}β₃ comprises a VH CDR3 having the amino acid sequence of SEQ ID NO:3 and a VL CDR2 having the amino acid sequence of SEQ ID NO:5. In a preferred embodiment, an antibody that immunospecifically binds to integrin α_{V}β₃ comprises a VH CDR3 having the amino acid sequence of SEQ ID NO:3 and a VL CDR3 having the amino acid sequence of SEQ ID NO:6.

The present invention also provides for a nucleic acid molecule, generally isolated, encoding an antibody that immunospecifically binds to integrin α_{V}β₃. In a specific embodiment, an isolated nucleic acid molecule encodes an antibody that immunospecifically binds to integrin α_{V}β₃ said antibody having the amino acid sequence of LM609 or VITAXIN™.

In one embodiment, an isolated nucleic acid molecule encodes an antibody that immunospecifically binds to integrin α_{V}β₃ said antibody comprising a VH domain having the amino acid sequence of the VH domain of LM609 or VITAXIN™. In another embodiment, an isolated nucleic acid molecule encodes an antibody that immunospecifically binds to integrin α_{V}β₃, said antibody comprising a VH domain having the amino acid sequence of the VH domain of the monoclonal antibody produced by the cell line deposited with the ATCC® as Accession Number HB 9537. In another embodiment, an isolated nucleic acid molecule encodes an antibody that immunospecifically binds to integrin α_{V}β₃, said antibody comprising a VH CDR1 having the amino acid sequence of the VH CDR1 listed in Table 1. In another embodiment, an isolated nucleic acid molecule encodes an antibody that immunospecifically binds to integrin α_{V}β₃, said antibody comprising a VH CDR2 having the amino acid sequence of the VH CDR listed in Table 1. In yet another embodiment, an isolated nucleic acid molecule encodes an antibody that immunospecifically binds to integrin α_{V}β₃, said antibody comprising a VH CDR3 having the amino acid sequence of the VH CDR3 listed in Table 1.

In one embodiment, an isolated nucleic acid molecule encodes an antibody that immunospecifically binds to integrin α_{V}β₃said antibody comprising a VL domain having the amino acid sequence of the VL domain of LM609 or VITAXIN™. In another embodiment, an isolated nucleic acid molecule encodes an antibody that immunospecifically binds to integrin α_{V}β₃ said antibody comprising a VL domain having the amino acid sequence of the VL domain of the monoclonal antibody produced by the cell line deposited with the ATCC® as Accession Number HB 9537. In another embodiment, an isolated nucleic acid molecule encodes an antibody that immunospecifically binds to integrin α_{V}β₃, said antibody comprising a VL CDR1 having the amino acid sequence of the VL CDR1 listed in Table 1. In another embodiment, an isolated nucleic acid molecule encodes an antibody that immunospecifically bind to integrin α_{V}β₃ said antibody comprising a VL CDR2 having the amino acid sequence of the VL CDR2 listed in Table 1. In yet another embodiment, an isolated nucleic acid molecule encodes an antibody that immunospecifically binds to integrin α_{V}β₃ said antibody comprising a VL CDR3 having the amino acid sequence of the VL CDR3 listed in Table 1.

In another embodiment, an isolated nucleic acid molecule encodes an antibody that immunospecifically binds to integrin α_{V}β₃, said antibody comprising a VH domain having the amino acid sequence of the VH domain of LM609 or VITAXIN™ and a VL domain having the amino acid sequence of the VL domain of LM609 or VITAXIN™. In another embodiment, an isolated nucleic acid molecule encodes an antibody that immunospecifically binds to integrin α_{V}β₃ said antibody comprising a VH CDR1, a VL CDR1, a VH CDR2, a VL CDR2, a VH CDR3, a VL CDR3, or any combination thereof having an amino acid sequence listed in Table 1.

The present invention also provides antibodies that immunospecifically bind to integrin α_{V}β₃, said antibodies comprising derivatives of the VH domains, VH CDRs, VL domains, or VL CDRs described herein that immunospecifically bind to integrin α_{V}β₃, Standard techniques known to those of skill in the art can be used to introduce mutations in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (i.e., amino acid residues which are not critical for the antibody to immunospecifically bind to integrin α_{V}β₃). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains ( e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention provides for antibodies that immunospecifically bind to integrin α_{V}β₃, said antibodies comprising the amino acid sequence of LM609 or VITAXIN™ with one or more amino acid residue substitutions in the variable light (VL) domain and/or variable heavy (VH) domain. The present invention also provides for antibodies that immunospecifically bind to integrin α_{V}β₃ said antibodies comprising the amino acid sequence of LM609 or VITAXIN™ with one or more amino acid residue substitutions in one or more VL CDRs and/or one or more VH CDRs. The antibody generated by introducing substitutions in the VH domain, VH CDRs, VL domain and/or VL CDRs of LM609 or VITAXIN™ can be tested in vitro and in vivo, for example, for its ability to bind to integrin α_{V}β₃ (by, e.g., immunoassays including, but not limited to ELISAs and BIAcore), or for its ability to prevent, treat or ameliorate one or more symptoms associated with an autoimmune or inflammatory disorder.

In a specific embodiment, an antibody that immunospecifically binds to integrin α_{V}β₃ comprises a nucleotide sequence that hybridizes to the nucleotide sequence encoding the monoclonal antibody produced by the cell line deposited with the ATCC® as Accession Number HB 9537 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

In a specific embodiment, an antibody that immunospecifically binds to integrin α_{V}β₃ comprises a nucleotide sequence that hybridizes to the nucleotide sequence encoding the LM609 or VITAXIN™ under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to integrin α_{V}β₃ comprises an amino acid sequence of a VH domain or an amino acid sequence a VL domain encoded by a nucleotide sequence that hybridizes to the nucleotide sequence encoding the VH or VL domains of LM609 or VITAXIN™ under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, an antibody that immunospecifically binds to integrin α_{V}β₃, comprises an amino acid sequence of a VH CDR or an amino acid sequence of a VL CDR encoded by a nucleotide sequence that hybridizes to the nucleotide sequence encoding any one of the VH CDRs or VL CDRs listed in Table 1 under stringent conditions e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to integrin α_{V}β₃ comprises an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC® as Accession Number HB 9537 under stringent conditions e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to integrin α_{V}β₃ comprises an amino acid sequence of a VH CDR and an amino acid sequence of a VL CDR encoded by nucleotide sequences that hybridizes to the nucleotide sequences encoding any one of the VH CDRs and VL CDRs listed in Table 1 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to integrin α_{V}β₃, comprises an amino acid sequence of a VH CDR and an amino acid sequence of a VL CDR encoded by nucleotide sequences that hybridizes to the nucleotide sequences encoding the monoclonal antibody produced by the cell line deposited with the ATCC® as Accession Number HB 9537 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In a specific embodiment, an antibody that immunospecifically binds to integrin α_{V}β₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC® as Accession Number HB 9537. In another embodiment, an antibody that immunospecifically binds to integrin α_{V}β₃ comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of VITAXIN™.

In another embodiment, an antibody that immunospecifically binds to integrin α_{V}β₃, comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of VITAXIN™. In another embodiment, an antibody that immunospecifically binds to integrin α_{V}β₃ comprises an amino acid sequence of a VH domain that is at least 35%, atleast 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC® as Accession Number HB 9537.

In another embodiment, an antibody that immunospecifically binds to integrin α_{V}β₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 1. In another embodiment, an antibody that immunospecifically binds to integrin α_{V}β₃ comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC® as Accession Number HB 9537.

In another embodiment, an antibody that immunospecifically binds to integrin α_{V}β₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of VITAXIN™. In another embodiment, an antibody that immunospecifically binds to integrin α_{V}β₃ comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC® as Accession Number HB 9537.

In another embodiment, an antibody that immunospecifically binds to integrin α_{V}β₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 1. In another embodiment, an antibody that immunospecifically binds to integrin α_{V}β₃ comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC® as Accession Number HB 9537.

The present invention encompasses antibodies that compete with an antibody described herein for binding to integrin α_{V}β₃. In a specific embodiment, the present invention encompasses antibodies that compete with LM609 or an antigen-binding fragment thereof for binding to integrin α_{V}β₃. In a preferred embodiment, the present invention encompasses antibodies that compete with VITAXIN™ or an antigen-binding fragment thereof for binding to integrin α_{V}β₃.

The present invention also encompasses VH domains that compete with the VH domain of LM609 or VITAXIN™ for binding to integrin α_{V}β₃. The present invention also encompasses VL domains that compete with a VL domain of LM609 or VITAXIN™ for binding to integrin α_{V}β₃.

The present invention also encompasses VH CDRs that compete with a VH CDR listed in Table 1 for binding to integrin α_{V}β₃ or a VH CDR of the monoclonal antibody produced by the cell line deposited with the ATCC as Accession Number HB 9537 for binding to integrin α_{V}β₃. The present invention also encompasses VL CDRs that compete with a VL CDR listed in Table 1 for binding to integrin α_{V}β₃ or a VL CDR of the monoclonal antibody produced by the cell line deposited with the ATCC as Accession Number HB 9537 for binding to integrin α_{V}β₃.

Antibodies that immunospecifically bind to integrin α_{V}β₃ include derivatives that are modified, i. e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies that immunospecifically bind to integrin α_{V}β₃, said antibodies comprising a framework region known to those of skill in the art. Preferably, the fragment region of an antibody of the invention is human. In a specific embodiment, an antibody that immunospecifically binds to integrin α_{V}β₃ comprises the framework region of VITAXIN™.

The present invention also encompasses antibodies which immunospecifically bind to integrin α_{V}β₃, said antibodies comprising the amino acid sequence of VITAXIN™ with one or more mutations (e.g., one or more amino acid substitutions) in the framework regions. In certain embodiments, antibodies which immunospecifically bind to integrin α_{V}β₃ comprise the amino acid sequence of VITAXIN™ with one or more amino acid residue substitutions in the framework regions of the VH and/or VL domains.

The present invention also encompasses antibodies which immunospecifically bind to integrin α_{V}β₃, said antibodies comprising the amino acid sequence of VITAXIN™ with one or more mutations (e.g., one or more amino acid residue substitutions) in the variable and framework regions.

The present invention also provides for fusion proteins comprising an antibody that immunospecifically binds to integrin α_{V}β₃ and a heterologous polypeptide. Preferably, the heterologous polypeptide that the antibody is fused to is useful for targeting the antibody to platelets, monocytes, endothelial cells, and/or B cells.

### 25 5.1.1.1 Antibodies Having Increased Half-lives that Immunospecifically Bind to Integrin α_{V}β₃

The present invention provides for antibodies that immunospecifically bind to integrin α_{V}β₃ which have a extended half-life in vivo. In particular, the present invention provides antibodies that immunospecifically bind to integrin α_{V}β₃ which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the—or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/3463 1; and U.S. Pat. No. 6,277,375, each of which is incorporated herein by reference in its entirety.

### 5.1.1.2. Antibody Conjugates

The present invention encompasses antibodies or antigen-binding fragments thereof that immunospecifically bind to integrin α_{V}β₃ recombinantly fused or chemically conjugated (including both covalently and non-covalently conjugations) to a heterologous polypeptide (or a fragment thereof, preferably at least 5, at least 10, at least 20, at least 30, at least 40, at least 50, at least 60, at least 70, at least 80, at least 90 or at least 100 contiguous amino acids of the polypeptide) to generate fusion proteins. The fusion does not necessarily need to be direct, but may occur through linker sequences. For example, antibodies may be used to target heterologous polypeptides to particular cell types (e.g., platelets, endothelial cells, B cells, or monocytes), either in vitro or in vivo, by fusing or conjugating the antibodies to antibodies specific for particular cell surface receptors such as, e.g., CD11c, CD 14, CD 17, CD19, CD25, CD36, CD41, CD42, CD51, CD61, CD70, and CD78.

The present invention also encompasses antibodies or antigen-binding fragments thereof that immunospecifically bind to integrin α_{V}β₃ fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses antibodies or antigen-binding fragments thereof that immunospecifically bind to integrin α_{V}β₃, conjugated to an agent which has a potential therapeutic benefit. An antibody or an antigen-binding fragment thereof that immunospecifically binds to integrin α_{V}β₃ may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or an antigen-binding fragment thereof that immunospecifically binds to integrin α_{V}β₃ may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-a (“IFN-α”), interferon-β (“IFN-β”), nerve growth factor (“NGF”), platelet derived growth factor (“PDGF”), tissue plasminogen activator (“TPA”), an apoptotic agent, e.g, TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), IL-2, IL-6, IL-10, granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and Thorpe et al., 1982, Immunol. Rev. 62:119-58.

An antibody or an antigen-binding fragment thereof that immunospecifically binds to integrin α_{V}β₃ can be conjugated to a second antibody to form an antibody heteroconjugate as described by Segal in U.S. Pat. No. 4,676,980, which is incorporated herein by reference in its entirety.

Antibodies or antigen-binding fragments thereof that immunospecifically bind to integrin α_{V}β₃ may be attached to solid supports, which are particularly useful for the purification of cells such as platelets and endothelial cells. Such solid supports include, but are not limited to, glass, cellulose, polyacrylamide, nylon, polystyrene, polyvinyl chloride or polypropylene.

5.2. Agents Used in Combination with Integrin α_{V}β₃ Antagonists

The present invention provides compositions comprising one or more integrin α_{V}β₃ antagonists and one or more prophylactic or therapeutic agents other than integrin α_{V}β₃ antagonists, and methods for preventing, treating or ameliorating one or more symptoms associated with an inflammatory or autoimmune disorder in a subject comprising administering to said subject one or more of said compositions. Therapeutic or prophylactic agents include, but are not limited to, peptides, polypeptides, fusion proteins, nucleic acid molecules, small molecules, mimetic agents, synthetic drugs, inorganic molecules, and organic molecules. Any agent which is known to be useful, or which has been used or is currently being used for the prevention, treatment or amelioration of one or more symptoms associated with an inflammatory or autoimmune disorder can be used in combination with an integrin α_{V}β₃ antagonist in accordance with the invention described herein. Examples of such agents include, but are not limited to, dermatological agents for rashes and swellings (e.g., phototherapy (i.e., ultraviolet B radiation), photochemotherapy (e.g., PUVA) and topical agents such as emolliments, salicyclic acid, coal tar, topical steroids, topical corticosteroids, topical vitamin D3 analogs (e.g., calcipotriene), tazarotene, and topical retinoids), anti-inflammatory agents (e.g., corticosteroids (e.g., prednisone and hydrocortisone), glucocorticoids, steroids, non-steriodal anti-inflammatory drugs (e.g., aspirin, ibuprofen, diclofenac, and COX-2 inhibitors), beta-agonists, anticholinergic agents and methyl xanthines), immunomodulatory agents (e.g., small organic molecules, a T cell receptor modulators, cytokine receptor modulators, T-cell depleting agents, cytokine antagonists, monokine antagonists, lymphocyte inhibitors, or anti-cancer agents), gold injections, sulphasalazine, penicillamine, anti-angiogenic agents (e.g., angiostatin, TNF-α antagonists (e.g., anti-TNFα antibodies), and endostatin), dapsone, psoralens (e.g., methoxalen and trioxsalen), anti-malarial agents (e.g., hydroxychloroquine), anti-viral agents, and antibiotics (e.g., erythomycin and penicillin).

5.2.1. Immunomodulatory Agents

Any immunomodulatory agent well-known to one of skill in the art may be used in the methods and compositions of the invention. Immunomodulatory agents can affect one or more or all aspects of the immune response in a subject. Aspects of the immune response include, but are not limited to, the inflammatory response, the complement cascade, leukocyte and lymphocyte differentiation, proliferation, and/or effector function, monocyte and/or basophil counts, and the cellular communication among cells of the immune system. In certain embodiments of the invention, an immunomodulatory agent modulates one aspect of the immune response. In other embodiments, an immunomodulatory agent modulates more than one aspect of the immune response. In a preferred embodiment of the invention, the administration of an immunomodulatory agent to a subject inhibits or reduces one or more aspects of the subject's immune response capabilities. In a specific embodiment of the invention, the immunomodulatory agent inhibits or suppresses the immune response in a subject. In accordance with the invention, an immunomodulatory agent is not an integrin α_{V}β₃ antagonist. In certain embodiments, an immunomodulatory agent is not an anti-inflammatory agent. In other embodiments, an immunomodulatory agent is not a CD2 antagonist. In other embodiments, an immunomodulatory agent is not a CD2 binding molecule. In yet other embodiments, an immunomodulatory agent is not MEDI-507.

An immunomodulatory agent may be selected to interfere with the interactions between the T helper subsets (TH1 or TH2) and B cells to inhibit neutralizing antibody formation. An immunomodulatory agent may be selected to inhibit the interaction between TH1 cells and CTLs to reduce the occurrence of CTL-mediated killing. An immunomodulatory agent may be selected to alter (e.g., inhibit or suppress) the proliferation, differentiation, activity and/or function of the CD4⁺ and/or CD8⁺ T cells. For example, antibodies specific for T cells can be used as immunomodulatory agents to deplete, or alter the proliferation, differentiation, activity and/or function of CD4⁺ and/or CD8⁺ T cells.

Examples of immunomodulatory agents include, but are not limited to, proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methothrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steriods, mycophenolate mofetil, rapamycin (sirolimus), mizoribine, deoxyspergualin, brequinar, malononitriloamindes (e.g., leflunamide), T cell receptor modulators, and cytokine receptor modulators. For clarification regarding T cell receptor modulators and cytokine receptor modulators see Section 3.1. Examples of T cell receptor modulators include, but are not limited to, anti-T cell receptor antibodies (e.g., anti-CD4 antibodies (e.g., cM-T412 (Boeringer), IDEC-CE9.1® (IDEC and SKB), mAB 4162W94, Orthoclone and OKTcdr4a (Janssen-Cilag)), anti-CD3 antibodies, anti-CD5 antibodies (e.g., an anti-CD5 ricin-linked immunoconjugate), anti-CD7 antibodies (e.g., CHH-380 (Novartis)), anti-CD8 antibodies, anti-CD40 ligand monoclonal antibodies, anti-CD52 antibodies (e.g., CAMPATH 1H (Ilex)), anti-CD2 monoclonal antibodies) and CTLA4-immunoglobulin. In a specific embodiment, a T cell receptor modulator is a CD2 antagonist. In other embodiments, a T cell receptor modulator is not a CD2 antagonist. In another specific embodiment, a T cell receptor modulator is a CD2 binding molecule, preferably MEDI-507. In other embodiments, a T cell receptor modulator is not a CD2 binding molecule.

Examples of cytokine receptor modulators include, but are not limited to, soluble cytokine receptors (e.g., the extracellular domain of a TNF-α receptor or a fragment thereof, the extracellular domain of an IL-1β receptor or a fragment thereof, and the extracellular domain of an IL-6 receptor or a fragment thereof), cytokines or fragments thereof (e.g, interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF), anti-cytokine receptor antibodies (e.g., anti-IL-2 receptor antibodies, anti-IL-4 receptor antibodies, anti-IL-6 receptor antibodies, anti-IL-10 receptor antibodies, and anti-IL-12 receptor antibodies), anti-cytokine antibodies (e.g., anti-IFN receptor antibodies, anti-TNF-α antibodies, anti-IL-1β antibodies, anti-IL-6 antibodies, and anti-IL-12 antibodies). In a specific embodiment, a cytokine receptor modulator is IL-4, IL-10, or a fragment thereof. In another embodiment, a cytokine receptor modulator is an anti-IL-1β antibody, anti-IL-6 antibody, anti-IL-12 receptor antibody, anti-TNF-α antibody. In another embodiment, a cytokine receptor modulator is the extracellular domain of a TNF-α receptor or a fragment thereof. In certain embodiments, a cytokine receptor modulator is not a TNF-α antagonist.

In a preferred embodiment, proteins, polypeptides or peptides (including antibodies) that are utilized as immunomodulatory agents are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as immunomodulatory agents are human or humanized.

In accordance with the invention, one or more immunomodulatory agents are administered to a subject with an inflammatory or autoimmune disease prior to, subsequent to, or concomitantly with the therapeutic and/or prophylactic agents of the invention. Preferably, one or more immunomodulatory agents are administered to a subject with an inflammatory or autoimmune disease to reduce or inhibit one or more aspects of the immune response as necessary. Any technique well-known to one skilled in the art can be used to measure one or more aspects of the immune response in a particular subject, and thereby determine when it is necessary to administer an immunomodulatory agent to said subject. In a preferred embodiment, an absolute lymphocyte count of approximately 500 cells/mm³, preferably 600 cells/mm³, more 700 cells/mm³, and most preferably 800 cells/mm³ is maintained in a subject. In another preferred embodiment, a subject with an autoimmune or inflammatory disorder is not administered an immunomodulatory agent if their absolute lymphocyte count is 500 cells/mm³ or less, 550 cells/mm³ or less, 600 cells/mm³ or less, 650 cells/mm³ or less, 700 cells/mm³ or less, 750 cells/mm³ or less, or 800 cells/mm³ or less.

In a preferred embodiment, one or more immunomodulatory agents are administered to a subject with an inflammatory or autoimmune disease so as to transiently reduce or inhibit one or more aspects of the immune response. Such a transient inhibition or reduction of one or more aspects of the immune system can last for hours, days, weeks, or months. Preferably, the transient inhibition or reduction in one or more aspects of the immune response last for a few hours (e.g., 2 hours, 4 hours, 6 hours, 8 hours, 12 hours, 14 hours, 16 hours, 18 hours, 24 hours, 36 hours, or 48 hours), a few days (e.g., 3 days, 4 days, 5 days, 6 days, 7 days, or 14 days), or a few weeks (e.g., 3 weeks, 4 weeks, 5 weeks weeks). The transient reduction or inhibition of one or more aspects of the immune response enhances the prophylactic and/or therapeutic capabilities of an integrin α_{V}β₃ antagonist.

In one embodiment of the invention, an immunomodulatory agent that reduces or depletes T cells, preferably memory T cells, is administered to a subject with an inflammatory or autoimmune disease in accordance with the methods of the invention. See, e.g., U.S. Pat. No. 4,658,019. In another embodiment of the invention, an immunomodulatory agent that inactivates CD8⁺ T cells is administered to a subject with an inflammatory or autoimmune disease in accordance with the methods of the invention. In a specific embodiment, anti-CD8 antibodies are used to reduce or deplete CD8⁺ T cells.

Antibodies that interfere with or block the interactions necessary for the activation of B cells by TH (T helper) cells, and thus block the production of neutralizing antibodies, are useful as immunomodulatory agents in the methods of the invention. For example, B cell activation by T cells requires certain interactions to occur (Durie et al, Immunol. Today, 15(9):406-410 (1994)), such as the binding of CD40 ligand on the T helper cell to the CD40 antigen on the B cell, and the binding of the CD28 and/or CTLA4 ligands on the T cell to the B7 antigen on the B cell. Without both interactions, the B cell cannot be activated to induce production of the neutralizing antibody.

The CD40 ligand (CD40L)-CD40 interaction is a desirable point to block the immune response because of its broad activity in both T helper cell activation and function as well as the absence of redundancy in its signaling pathway. Thus, in a specific embodiment of the invention, the interaction of CD40L with CD40 is transiently blocked at the time of administration of one or more of the immunomodulatory agents. This can be accomplished by treating with an agent which blocks the CD40 ligand on the TH cell and interferes with the normal binding of CD40 ligand on the T helper cell with the CD40 antigen on the B cell. An antibody to CD40 ligand (anti-CD40L) (available from Bristol-Myers Squibb Co; see, e.g., European patent application 555,880, published Aug. 18, 1993) or a soluble CD40 molecule can be selected and used as an immunomodulatory agent in accordance with the methods of the invention.

In another embodiment, an immunomodulatory agent which reduces or inhibits one or more biological activities (e.g., the differentiation, proliferation, and/or effector functions) of TH0, TH1, and/or TH2 subsets of CD4⁺ T helper cells is administered to a subject with an inflammatory or autoimmune disease in accordance with the methods of the invention. One example of such an immunomodulatory agent is IL-4. IL-4 enhances antigen-specific activity of TH2 cells at the expense of the THI cell function (see, e.g., Yokota et al, 1986 Proc. Natl. Acad. Sci., USA, 83:5894-5898; and U.S. Pat. No. 5,017,691). Other examples of immunomodulatory agents that affect the biological activity (e.g., proliferation, differentiation, and/or effector functions) of T-helper cells (in particular, THI and/or TH2 cells) include, but are not limited to, IL-6, IL-10, IL-12, and interferon (IFN)-γ.

In another embodiment, an immunomodulatory agent administered to a subject with an inflammatory or autoimmune disease in accordance with the methods of the invention is a cytokine that prevents antigen presentation. In a preferred embodiment, an immunomodulatory agent used in the methods of the invention is IL-10. IL-10 also reduces or inhibits macrophage action which involves bacterial elimination.

Other examples of immunomodulatory agents which can be used in accordance with the invention include, but are not limited to, corticosteroids, azathioprine, mycophenolate mofetil, cyclosporin A, hydrocortisone, FK506, methotrexate, leflunomide, and cyclophosphamide. A short course of cyclophosphamide has been demonstrated to successfully interrupt both CD4⁺ and CD8⁺ T cell activation to adenoviral capsid protein (Jooss et al., 1996, Hum. Gene Ther. 7:1555-1566), and at higher doses, formation of neutralizing antibody was prevented. Hydrocortisone or cyclosporin A treatment has been successfully used to decrease the induction of cytokines, some of which may be involved in the clearance of bacterial infections.

Nucleic acid molecules encoding proteins, polypeptides, or peptides with immunomodulatory activity or proteins, polypeptides, or peptides with immunomodulatory activity can be administered to a subject with an inflammatory or autoimmune disease in accordance with the methods of the invention. Further, nucleic acid molecules encoding derivatives, analogs, fragments or variants of proteins, polypeptides, or peptides with immunomodulatory activity, or derivatives, analogs, fragments or variants of proteins, polypeptides, or peptides with immunomodulatory activity can be administered to a subject with an inflammatory or autoimmune disease in accordance with the methods of the invention. Prefereably, such derivatives, analogs, variants and fragments retain the immunomodulatory activity of the full-length wild-type protein, polypeptide, or peptide.

Proteins, polypeptides, or peptides that can be used as immunomodulatory agents can be produced by any technique well-known in the art or described herein. See, e.g., Chapter 16 Ausubel et al. (eds.), 1999, Short Protocols in Molecular Biology, Fourth Edition, John Wiley & Sons, NY, which describes methods of producing proteins, polypeptides, or peptides, and which is incorporated herein by reference in its entirety. Antibodies which can be used as immunomodulatory agents can be produced by, e.g., methods described in U.S. Pat. No. 6,245,527 and in Harlow and Lane Antibodies: A Laboratory Manual, Cold Spring Harbor Laboratory Press, Cold Spring Harbor, N.Y., 1988, which are incorporated herein by reference in their entirety. Preferably, agents that are commercially available and known to function as immunomoulatory agents are used in the compositions and methods of the invention. The immunomodulatory activity of an agent can be determined in vitro and/or in vivo by any technique well-known to one skilled in the art, including, e.g., by CTL assays, proliferation assays, and immunoassays (e.g. ELISAs) for the expression of particular proteins such as co-stimulatory molecules and cytokines.

5.2.2. CD2 Antagonists

In certain embodiments, CD2 antagonists directly or indirectly the depletion of peripheral blood lymphocytes, preferably T lymphocytes and/or NK cells. In other embodiments, a CD2 antagonist inhibits T-cell proliferation by at least 25%, at least 30%, at least 35%, at least 40%, at least 50%, at least 55%, at least 60%, at least 65%, at least 70at least 75%, at least 80%, at least 85%, at least 90%, at least 95%, or at least 98% in an in vivo or in vitro assay described herein or known to one of skill in the art. In other embodiments, a CD2 antagonist induces cytolysis of T-cells. In other embodiments, a CD2 antagonist inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art. In yet other embodiments, a CD2 binding antagonist inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In certain embodiments a CD2 antagonist inhibits or reduces the interaction between a CD2 polypeptide and LFA-3 by at least 25%, at least 30%, at least 35%, at least 40%, at least 50%, at least 55%, at least 60%, at least 65%, at least 70%, at least 75%, at least 80% at least 85%, at least 90%, at least 95%, or at least 98% in an in vivo or in vitro assay described herein (e.g., an ELISA) or known to one of skill in the art. In other embodiments, a CD2 antagonist does not inhibit the interaction between a CD2 polypeptide and LFA-3. In yet other embodiments, a CD2 antagonist inhibits the interaction between a CD2 polypeptide and LFA-3 by less than 20%, less 15%, less than 10%, or less than 5%.

In certain embodiments, a CD2 antagonist does not induce or reduces cytokine expression and/or release in an in vivo or in vitro assay described herein or well-known to one of skill in the art. In a specific embodiment, a CD2 antagonist does not induce an increase in the concentration of cytokines such as, e.g., interferon-γ (“IFN-γ”), interleukin-2 (“IL-2”), interleukin-4 (“IL-4”), interleukin-6 (“IL-6”), interleukin-9 (“IL-9”), interleukin-12 (“IL-12”), and interleukin-15 (“IL-15”) in the serum of a subject administered a CD2 antagonist. In alternative embodiments, a CD2 antagonist induces cytokine expression and/or release in an in vitro or in vivo assay described herein or known to one of skill in the art. In a specific embodiment, a CD2 antagonist induces an increase in the concentration of cytokines such as, e.g., IFN-γ, IL-2, IL4, IL-6, interleukin-7 (“IL-7”), IL-9, interleukin-10 (“IL-10”), and tumor necrosis factor a (“TNF-γ”) in the serum of a subject administered a CD2 binding molecule. Serum concentrations of cytokines can be measured by any technique well-known to one of skill in the art such as immunoassays, including, e.g., ELISA.

In certain embodiments, a CD2 antagonist induces T-cell anergy in an in vivo or in vitro assay described herein or known to one of skill in the art. In alternative embodiments, a CD2 antagonist does not induce T-cell anergy in an in vivo or in vitro assay described herein or known to one of skill in the art. In other embodiments, a CD2 antagonist elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In other embodiments, a CD2 antagonist inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art.

In certain embodiments, a CD2 antagonist is not a small organic molecule. In other embodiments, a CD2 antagonist is not an antisense nucleic acid molecule or triple helix. In a preferred embodiment, a CD2 antagonist is a CD2 binding molecule.

In a preferred embodiment, proteins, polypeptides or peptides (including antibodies and fusion proteins) that are utilized as CD2 antagonists are derived from the same species as the recipient of the proteins, polypeptides or peptides so as to reduce the likelihood of an immune response to those proteins, polypeptides or peptides. In another preferred embodiment, when the subject is a human, the proteins, polypeptides, or peptides that are utilized as CD2 antagonists are human or humanized.

Nucleic acid molecules encoding proteins, polypeptides, or peptides that function as CD2 antagonists, or proteins, polypeptides, or peptides that function as CD2 antagonists can be administered to a subject with an inflammatory or autoimmune disorder in accordance with the methods of the invention. Further, nucleic acid molecules encoding derivatives, analogs, fragments or variants of proteins, polypeptides, or peptides that function as CD2 antagonists, or derivatives, analogs, fragments or variants of proteins, polypeptides, or peptides that function as CD2 antagonists can be administered to a subject with an inflammatory or autoimmune disorder in accordance with the methods of the invention. Preferably, such derivatives, analogs, variants and fragments retain the CD2 antagonist activity of the full-length wild-type protein, polypeptide, or peptide.

5.2.3. CD2 Binding Molecules

The term “CD2 binding molecule” and analogous terms, as used herein, refer to a bioactive molecule that immunospecifically binds to a CD2 polypeptide and directly or indirectly modulate an activity or function of lymphocytes, in particular, peripheral blood T-cells. In a specific embodiment, CD2 binding molecules directly or indirectly mediate the depletion of lymphocytes, in particular peripheral blood T-cells. Preferably, the CD2 binding molecule binds to a CD2 polypeptide and preferentially mediates depletion of memory T cells (i.e., CD45RO⁺ T cells) and not naive T cells. In a specific embodiment, a CD2 binding molecule immunospecifically binds a CD2 polypeptide expressed by an immune cell such as a T-cell or NK cell. In a preferred embodiment, a CD2 binding molecule immunospecifically binds a CD2 polypeptide expressed by a T-cell and/or NK cell. CD2 binding molecules can be identified, for example, by immunoassays or other techniques well-known to those of skill in the art. CD2 binding molecules include, but are not limited to, peptides, polypeptides, fusion proteins, small molecules, mimetic agents, synthetic drugs, organic molecules, inorganic molecules, and antibodies.

In one embodiment, a CD2 binding molecule mediates depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In another embodiment, a CD2 binding molecule mediates depletion of peripheral blood T-cells by inducing cytolysis of T-cells. In yet another embodiment, a CD2 binding molecule mediates depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art.

In a specific embodiment, a CD2 binding molecule immunospecifically binds to a CD2 polypeptide and does not non-specifically bind to other polypeptides. In another embodiment, a CD2 binding molecule immunospecifically binds to a CD2 polypeptide and has cross-reactivity with other antigens. In a preferred embodiment, a CD2 binding molecule immunospecifically binds to a CD2 polypeptide and does not cross-react with other antigens.

In one embodiment, a CD2 binding molecule inhibits or reduces the interaction between a CD2 polypeptide and a naturally occurring in vivo CD2 binding partner (e.g., an LFA-3 molecule) by approximately 25%, 30%, 35%, 40%, 45%, 50%, 55%, 60%, 65%, 70%, 75%, 80%, 85%, 90%, 95%, or 98% in an in vivo or in vitro assay described herein or well-known to one of skill in the art. In an alternative embodiment, a CD2 binding molecule does not inhibit the interaction between a CD2 polypeptide and a naturally occurring in vivo CD2 binding partner (e.g, LFA-3 molecule) in an in vivo or in vitro assay described herein or known to one of skill in the art. In another embodiment, a CD2 binding molecule inhibits the interaction between a CD2 polypeptide and LFA-3 by less than 20%, less than 15%, less than 10%, or less than 5%. A naturally occurring in vivo CD2 binding partner includes, but is not limited to, a peptide, a polypeptide, and an organic molecule that binds to a CD2 polypeptide. Preferably, a naturally occurring in vivo CD2 binding partner binds to the extracellular domain or a fragment thereof of a CD2 polypeptide.

In a specific embodiment, a CD2 binding molecule inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art.

In another embodiment, a CD2 binding molecule does not induce or reduces cytokine expression and/or release in an in vivo or in vitro assay described herein or well-known to one of skill in the art. In a specific embodiment, a CD2 binding molecule does not induce an increase in the concentration of cytokines such as, e.g., interferon-y (“IFN-γ”), interleukin-2 (“IL-2”), interleukin-4 (“IL-4”), interleukin-6 (“IL-6”), interleukin-9 (“IL-9”), interleukin-12 (“IL-12”), and interleukin-15 (“IL-15”) in the serum of a subject administered a CD2 binding molecule. In an alternative embodiment, a CD2 binding molecule induces cytokine expression and/or release in an in vitro or in vivo assay described herein or known to one of skill in the art. In a specific embodiment, a CD2 binding molecule induces an increase in the concentration of cytokines such as, e.g., IFN-γ, IL-2, IL4, IL-6, interleukin-7 (“IL-7”), IL-9, interleukin-10 (“IL-10”), and tumor necrosis factor a (“TNF-α”) in the serum of a subject administered a CD2 binding molecule. Serum concentrations of cytokines can be measured by any technique well-known to one of skill in the art such as immunoassays, including, e.g, ELISA.

In a specific embodiment, a CD2 binding molecule induces T-cell anergy in an in vivo or in vitro assay described herein or known to one of skill in the art. In an alternative embodiment, a CD2 binding molecule does not induce T-cell anergy in an in vivo or in vitro assay described herein or known to one of skill in the art. In another embodiment, a CD2 binding molecule elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In another embodiment, a CD2 binding molecule inhibits T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibits T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art.

In one embodiment, a CD2 binding molecule is an antibody or antigen-binding fragment thereof that immunospecifically binds to a CD2 polypeptide. In a preferred embodiment, a CD2 binding molecule is an antibody or an antigen-binding fragment thereof that immunospecifically binds to a CD2 polypeptide expressed by an immune cell such as a T-cell or NK cell. In another embodiment, a CD2 binding molecule is a peptide, a mimetic agent, an inorganic molecule or an organic molecule that immunospecifically binds to a CD2 polypeptide. In another embodiment, a CD2 binding molecule is an LFA-3 peptide, polypeptide, derivative, or analog thereof that immunospecifically binds to a CD2 polypeptide. In another embodiment, a CD2 binding molecule is a fusion protein that immunospecifically binds to a CD2 polypeptide. In a preferred embodiment, a CD2 binding molecule is a fusion protein that immunospecifically binds to a CD2 polypeptide expressed by an immune cell such as a T-cell or NK cell. In certain embodiments, a CD2 binding molecule is a small organic molecule. In other embodiments, a CD2 binding molecule is not an organic molecule.

5.2.3.1. Antibodies that Immunospecifically Bind to CD2 Polvpeptides

It should be recognized that antibodies that immunospecifically bind to a CD2 polypeptide are known in the art. Examples of known antibodies that immunospecifically bind to a CD2 polypeptide include, but are not limited to, the murine monoclonal antibody produced by the cell line UMCD2 (Ancell Immunology Research Products, Bayport, Minn.; Kozarsky et al., 1993, Cell Immunol. 150:235-246), the murine monoclonal antibody produced by cell line RPA2.10 (Zymed Laboratories, Inc., San Francisco, Calif.; Rabinowitz et al., Clin. Immunol. & Immunopathol. 76(2):148-154), the rat monoclonal antibody LO-CD2b (International Publication No. WO 00/78814 A2), the rat monoclonal antibody LO-CD2a/BTI-322 (Latinne et al., 1996, Int. Immunol. 8(7):1113-1119), and the humanized monoclonal antibody MEDI-507 (MedImmune, Inc., Gaithersburg, MD; Branco et al., 1999, Transplantation 68(10): 1588-1596).

The present invention provides antibodies that immunospecifically bind to a CD2 polypeptide expressed by an immune cell such as a T-cell or NK cell, and said antibodies modulate an activity or function of lymphocytes, preferably peripheral blood T-cells. In a specific embodiment, antibodies that immunospecifically bind to a CD2 polypeptide directly or indirectly meditate the depletion of lymphocytes, preferably peripheral blood T-cells. In particular, the present invention provides antibodies that immunospecifically bind to a CD2 polypeptide expressed by a T-cell and/or NK cell, and said antibodies mediate depletion of peripheral blood T-cells.

In a specific embodiment, antibodies that immunospecifically bind to a CD2 polypeptide inhibit or reduce the interaction between a CD2 polypeptide and LFA-3 by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. In an alternative embodiment, antibodies that immunospecifically bind to a CD2 polypeptide do not inhibit the interaction between a CD2 polypeptide and LFA-3 in an in vivo or in vitro assay described herein or well-known to one of skill in the art. In another embodiment, antibodies that immunospecifically bind to a CD2 polypeptide inhibit the interaction between a CD2 polypeptide and LFA-3 by less than 20%, less than 15%, less than 10%, or less than 5%.

In a specific embodiment, antibodies that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, antibodies that immunospecifically bind to a CD2 polypeptide do not induce or reduce cytokine expression and/or release in an in vivo or in vitro assay described herein or well-known to one of skill in the art. In a specific embodiment, antibodies that immunospecifically bind to a CD2 polypeptide do not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, antibodies that immunospecifically binds to a CD2 polypeptide induce cytokine expression and/or release in an in vitro or in vivo assay described herein or well-known to one of skill in the art. In a specific embodiment, an antibody that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g, ELISA.

In another embodiment, antibodies that immunospecifically bind to a CD2 polypeptide induce T-cell anergy in an in vivo or in vitro assay described herein or well-known to one of skill in the art. In an alternative embodiment, antibodies that immunospecifically bind to a CD2 polypeptide do not induce T-cell anergy in an in vivo or in vitro assay described herein or well-known to one of skill in the art. In another embodiment, antibodies that immunospecifically bind to a CD2 polypeptide elicit a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or known to one of skill in the art.

In one embodiment, antibodies that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art. In another embodiment, antibodies that immunospecifically bind to a CD2 polypeptide mediate depletion of peripheral blood T-cells by inhibiting T-cell proliferation by inducing cytolysis of T-cells. In yet another embodiment, antibodies that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or well-known to one of skill in the art.

In another embodiment, antibodies that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, the Fc domain of an antibody that immunospecifically binds to a CD2 polypeptide binds to an Fc receptor (“FcR”) expressed by an immune cell such as an NK cell, a monocyte, and macrophage. In a preferred embodiment, the Fc domain of an antibody that immunospecifically binds to a CD2 polypeptide binds to an FcγRIII expressed by an immune cell such as an NK cell, a monocyte, and a macrophage. In another embodiment, a fragment of the Fc domain (e.g., the CH2 and/or CH3 region of the Fc domain) of an antibody that immunospecifically binds to a CD2 polypeptide binds to an FcR expressed by an immune cell such as an NK cell, a monocyte, and a macrophage.

Antibodies that immunospecifically bind to a CD2 polypeptide include, but are not limited to, monoclonal antibodies, multispecific antibodies, human antibodies, humanized antibodies, chimeric antibodies, single-chain Fvs (scFv), single chain antibodies, Fab fragments, F(ab′) fragments, disulfide-linked Fvs (sdFv), and anti-idiotypic (anti-Id) antibodies (including, e.g., anti-Id antibodies to antibodies of the invention), and epitope-binding fragments of any of the above. In particular, antibodies that immunospecifically bind to a CD2 polypeptide include immunoglobulin molecules and immunologically active portions of immunoglobulin molecules, i.e., molecules that contain an antigen binding site that immunospecifically binds to a CD2 polypeptide. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule. In a specific embodiment, the antibodies that immunospecifically bind to a CD2 polypeptide and mediate the depletion of T-cells comprise an Fc domain or a fragment thereof (e.g., the CH2, CH3, and/or hinge regions of an Fc domain). In a preferred embodiment, the antibodies that immunospecifically bind to a CD2 polypeptide and mediate the depletion of T cells comprise an Fc domain or fragment thereof that binds to an FcR, preferably an FcγRIII, expressed by an immune cell.

The antibodies that immunospecifically bind to a CD2 polypeptide may be from any animal origin including birds and mammals (e.g., human, murine, donkey, sheep, rabbit, goat, guinea pig, camel, horse, or chicken). Preferably, the antibodies of the invention are human or humanized monoclonal antibodies. Human antibodies that immunospecifically bind to a CD2 polypeptide include antibodies having the amino acid sequence of a human immunoglobulin and antibodies isolated from human immunoglobulin libraries or from mice that express antibodies from human genes.

The antibodies that immunospecifically bind to a CD2 polypeptide may be monospecific, bispecific, trispecific or of greater multispecificity. Multispecific antibodies may be specific for different epitopes of a CD2 polypeptide or may be specific for both a CD2 polypeptide as well as for a heterologous epitope, such as a heterologous polypeptide or solid support material. See, e.g., PCT publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

The present invention provides for antibodies that have a high binding affinity for a CD2 polypeptide. In a specific embodiment, an antibody that immunospecifically binds to a CD2 polypeptide has an association rate constant or kₒₙ rate (antibody (Ab)+antigen (Ag)^{k}^{ᵢₙ}→Ab−Ag)ofatleast 10⁵M⁻¹s⁻¹, at least 5×10⁵ M⁻¹s⁻¹, at least 10⁶ M⁻¹s^{31 1}, at least 5×10⁶ M⁻¹s⁻¹, at least 10⁷M⁻¹s⁻¹, at least 5×10⁷M⁻¹s⁻¹, or at least 10⁸ M⁻¹s⁻¹. In a preferred embodiment, an antibody that immunospecifically binds to a CD2 polypeptide has a kₒₙ, of at least 2×10⁵M⁻¹s⁻¹, at least 5×10⁵ M⁻¹s⁻¹, at least 10⁶M⁻¹s⁻¹, at least 5×10⁶ M⁻¹s⁻¹, at least 10⁷M⁻¹s⁻¹, at least 5×10⁷ M⁻¹s⁻¹, or at least 10⁸M⁻¹s⁻¹.

In another embodiment, an antibody that immunospecifically binds to a CD2 polypeptide has a k_{off} rate (antibody (Ab)+antigen (Ag)^{k}^{_{off}}←Ab−Ag) of less than 10⁻¹ s⁻¹, less than 5×10⁻¹ s⁻¹, less than 10⁻² s⁻¹, less than 5×10⁻² s⁻¹, less than 10⁻³ s⁻¹, less than 5×10⁻³ s⁻¹, less than 10⁻⁴ s⁻¹, less than 5×10⁻⁴ s⁻¹, less than 10⁻⁵ s⁻¹, less than 5×10⁻⁵ s⁻¹, less than 10⁻⁶ s⁻¹, less than 5×10⁻⁶ s⁻¹, less than 10⁻⁷ s⁻¹, less than 5×10⁻⁷ s⁻¹, less than 10⁻⁸ s⁻¹, less than 5×10⁻⁸s^{31 1}, less than 10⁻⁹ s⁻¹, less than 5×10⁻⁹ s⁻¹, or less than 10⁻¹⁰ s⁻¹. In a preferred embodiment, an antibody that immunospecifically binds to a CD2 polypeptide has a kₒₙ of less than 5×10⁻⁴ s⁻¹, less than 10⁻⁵ s⁻¹, less than 5×10⁻⁵ s⁻¹, less than 10⁻⁶s⁻¹, less than 5×10⁻⁶ s⁻¹, less than 10⁻⁷ s^{31 1}, less than 5×10⁻⁷ s⁻¹, less than 10⁻⁸ s⁻¹, less than 5×10⁻⁸ s⁻¹, less than 10⁻⁹ s⁻¹, less than 5×10⁻⁹ s⁻¹, less than 10⁻¹⁰ s⁻¹.

In another embodiment, an antibody that immunospecifically binds to a CD2 polypeptide has an affinity constant or Kₐ (kₒₙ/k_{off}) of at least 10² M⁻¹, at least 5×10² M⁻¹, at least 10³ M⁻¹, at least 5×10³ M⁻¹, at least 10⁴ M⁻¹, at least 5×10⁴ M⁻¹, at least 10⁵ M⁻¹, at least 5×10⁵ M⁻¹, at least 10⁶ M⁻¹, at least 5×10⁶ M⁻¹, at least 10⁷ M⁻¹, at least 5×10⁷ M⁻¹, at least 10⁸ M⁻¹, at least 5×10⁸ M⁻¹, at least 10⁹ M⁻¹, at least 5×10⁹ M⁻¹, at least 10¹⁰ M⁻¹, at least 5×10¹⁰ M⁻¹, at least 10¹¹ M⁻¹, at least 5×10¹¹ M⁻¹, at least 10¹² M⁻¹, at least 5×10¹² M⁻¹, at least 10¹³M⁻¹, at least 5×10¹³ M⁻¹, at least 10¹⁴ M⁻¹, at least 5×10¹⁴ M⁻¹, at least 10¹⁵ M⁻¹, or at least 5×10¹⁵ M⁻¹. In yet another embodiment, an antibody that immunospecifically binds to a CD2 polypeptide has a dissociation constant or K_{d} (k_{off}/kₒₙ) of less than 10⁻² M, less than 5×10⁻² M, less than 10⁻³ M, less than 5×10⁻³ M, less than 10⁻⁴ M, less than 5×10⁻⁴ M, less than 10⁻⁵ M, less than 5×10⁻⁵ M, less than 10⁻⁶ M, less than 5×10⁻⁶ M, less than 10⁻⁷ M, less than 5×10⁻⁷ M, less than 10⁻⁸ M, less than 5×10⁻⁸ M, less than 10⁻⁹ M, less than 5×10⁻⁹ M, less than 10⁻¹⁰ M, less than 5×10⁻¹⁰ M, less than 10⁻¹¹ M, less than 5×10⁻¹¹ M, less than 10⁻¹² M, less than 5×10⁻¹² M, less than 10⁻¹³ M, less than 5×10⁻¹³ M, less than 10⁻¹⁴ M, less than 5×10⁻¹⁴ M, less than 10⁻¹⁵ M, or less than 5×10⁻¹⁵ M.

In a specific embodiment, an antibody that immunospecifically binds to a CD2 polypeptide is LO-CD2a/BTI-322 or an antigen-binding fragment thereof e.g., (one or more complementarity determining regions (CDRs) of LO-CD2a/BTI-322). LO-CD2a/BTI-322 has the amino acid sequence disclosed, e.g., in U.S. Pat. Nos. 5,730,979, 5,817,311, and 5,951,983; and U.S. application Ser. Nos. 09/056,072 and 09/462,140 (each of which is incorporated herein by reference in its entirety), or the amino acid sequence of the monoclonal antibody produced by the cell line deposited with the American Type Culture Collection (ATCC®), 10801 University Boulevard, Manassas, Va. 20110-2209 on Jul. 28, 1993 as Accession Number HB 11423. In an alternative embodiment, an antibody that immunospecifically binds to a CD2 polypeptide is not LO-CD2a/BTI-322 or an antigen-binding fragment of LO-CD2a/BTI-322.

In another specific embodiment, an antibody that immunospecifically binds to a CD2 polypeptide is LO-CD2b or an antigen-binding fragment thereof (e.g., one or more CDRs of LO-CD2b). LO-CD2b has the amino acid sequence of the antibody produced by the cell line deposited with the ATCC®, 10801 University Boulevard, Manassas, Va. 20110-2209 on Jun. 22, 1999 as Accession Number PTA-802, or disclosed in, e.g., Dehoux et al., 2000, Transplantation 69(12):2622-2633 and International Publication No. WO 00/78814 (each of which is incorporated herein by reference in its entirety). In an alternative embodiment, an antibody that immunospecifically binds to a CD2 polypeptide is not LO-CD2b or an antigen-binding fragment of LO-CD2b.

In a preferred embodiment, an antibody that immunospecifically binds to a CD2 polypeptide is MEDI-507 or an antibody-binding fragment thereof (e.g., one or more CDRs of MEDI-507). MEDI-507 is disclosed, e.g., in PCT Publication No. WO 99/03502 and U.S. application Ser. No. 09/462,140, each of which is incorporated herein by reference in its entirety. In an alternative embodiment, an antibody of the present invention is not MEDI-507 or an antigen-binding fragment of MEDI-507.

The present invention also provides antibodies that immunospecifically bind a CD2 polypeptide, said antibodies comprising a variable heavy (“VH”) domain having an amino acid sequence of the VH domain for LO-CD2a/BTI-322 or MEDI-507. The present invention also provides antibodies that immunospecifically bind to a CD2 polypeptide, said antibodies comprising a VH CDR having an amino acid sequence of any one of the VH CDRs listed in Table 2.

TABLE 2
CDR Sequences Of LO-CD2a/BTI-322

| CDR | Sequence | SEQ ID NO: |
|---|---|---|
| VH1 | EYYMY | 11 |
| VH2 | RIDPEDGSIDYVEKFKK | 12 |
| VH3 | GKFNYRFAY | 13 |
| VL1 | RSSQSLLHSSGNTLNW | 14 |
| VL2 | LVSKLES | 15 |
| VL3 | MQFTHYPYT | 16 |

In one embodiment, antibodies that immunospecifically bind to a CD2 polypeptide comprise a VH CDR1 having the amino acid sequence of SEQ ID NO:11. In another embodiment, antibodies that immunospecifically bind to a CD2 polypeptide comprise a VH CDR2 having the amino acid sequence of SEQ ID NO:12. In another embodiment, antibodies that immunospecifically bind to a CD2 polypeptide comprise a VH CDR3 having the amino acid sequence of SEQ ID NO:13. In a preferred embodiment, antibodies that immunospecifically bind to a CD2 polypeptide comprise a VH CDR1 having the amino acid sequence of SEQ ID NO:11, a VH CDR2 having the amino acid sequence of SEQ ID NO:12, and a VH CDR3 having the amino acid sequence of SEQ ID NO:13.

The present invention also provides antibodies that immunospecifically bind to a CD2 polypeptide, said antibodies comprising a variable light (“VL”) domain having an amino acid sequence of the VL domain for LO-CD2a/BTI-322 or MEDI-507. The present invention also provides antibodies that immunospecifically bind to a CD2 polypeptide, said antibodies comprising a VL CDR having an amino acid sequence of any one of the VL CDRs listed in Table 2.

In one embodiment, antibodies that immunospecifically bind to a CD2 polypeptide comprise a VL CDR1 having the amino acid sequence of SEQ ID NO:14. In another embodiment, antibodies that immunospecifically bind to a CD2 polypeptide comprise a VL CDR2 having the amino acid sequence of SEQ ID NO:15. In another embodiment, antibodies that immunospecifically bind to a CD2 polypeptide comprise a VL CDR3 having the amino acid sequence of SEQ ID NO:16. In a preferred embodiment, antibodies that immunospecifically bind to a CD2 polypeptide comprise a VL CDR1 having the amino acid sequence of SEQ ID NO:14, a VL CDR2 having the amino acid sequence of SEQ ID NO:15, and a VL CDR3 having the amino acid sequence of SEQ ID NO:16.

The present invention also provides antibodies that immunospecifically bind to a CD2 polypeptide, said antibodies comprising a VH domain disclosed herein combined with a VL domain disclosed herein, or other VL domain. The present invention further provides antibodies that immunospecifically bind to a CD2 polypeptide, said antibodies comprising a VL domain disclosed herein combined with a VH domain disclosed herein, or other VH domain.

The present invention also provides antibodies that immunospecifically bind to a CD2 polypeptide, said antibodies comprising one or more VH CDRs and one or more VL CDRs listed in Table 2. In particular, the invention provides for an antibody that immunospecifically binds to a CD2 polypeptide, said antibody comprising a VH CDR1 and a VL CDR1, a VH CDR1 and a VL CDR2, a VH CDR1 and a VL CDR3, a VH CDR and a VL CDR1, VH CDR2 and VL CDR2, a VH CDR2 and a VL CDR3, a VH CDR3 and a VH CDR1, a VH CDR3 and a VL CDR2, a VH CDR3 and a VL CDR3, or any combination thereof of the VH CDRs and VL CDRs listed in Table 2.

In one embodiment, an antibody that immunospecifically binds to a CD2 polypeptide comprises a VH CDR1 having the amino acid sequence of SEQ ID NO:11 and a VL CDR1 having the amino acid sequence of SEQ ID NO:14. In another embodiment, an antibody that immunospecifically binds to a CD2 polypeptide comprises a VH CDR1 having the amino acid sequence of SEQ ID NO:11 and a VL CDR2 having the amino acid sequence of SEQ ID NO:15. In another embodiment, an antibody that immunospecifically binds to a CD2 polypeptide comprises a VH CDR1 having the amino acid sequence of SEQ ID NO:11 and a VL CDR3 having the amino acid sequence of SEQ ID NO:16.

In another embodiment, an antibody that immunospecifically binds to a CD2 polypeptide comprises a VH CDR2 having the amino acid sequence of SEQ ID NO:2 and a VL CDR1 having the amino acid sequence of SEQ ID NO:14. In another embodiment, an antibody that immunospecifically binds to a CD2 polypeptide comprises a VH CDR2 having the amino acid sequence of SEQ ID NO:12 and a VL CDR2 having the amino acid sequence of SEQ ID NO:15. In another embodiment, an antibody that immunospecifically binds to a CD2 polypeptide comprises a VH CDR2 having the amino acid sequence of SEQ ID NO:12 and a VL CDR3 having the amino acid sequence of SEQ ID NO:16.

In another embodiment, an antibody that immunospecifically binds to a CD2 polypeptide comprises a VH CDR3 having the amino acid sequence of SEQ ID NO:13 and a VL CDR1 having the amino acid sequence of SEQ ID NO:14. In another embodiment, an antibody that immunospecifically binds to a CD2 polypeptide comprises a VH CDR3 having the amino acid sequence of SEQ ID NO:13 and a VL CDR2 having the amino acid sequence of SEQ ID NO:15. In a preferred embodiment, an antibody that immunospecifically binds to a CD2 polypeptide comprises a VH CDR3 having the amino acid sequence of SEQ ID NO:13 and a VL CDR3 having the amino acid sequence of SEQ ID NO:16.

The present invention also provides for a nucleic acid molecule, generally isolated, encoding an antibody that immunospecifically binds to a CD2 polypeptide. In a specific embodiment, an isolated nucleic acid molecule encodes an antibody that immunospecifically binds to a CD2 polypeptide, said antibody having the amino acid sequence of LO-CD2a/BTI-322, LO-CD2b, or MEDI-507.

In one embodiment, an isolated nucleic acid molecule encodes an antibody that immunospecifically binds to a CD2 polypeptide, said antibody comprising a VH domain having the amino acid sequence of the VH domain of LO-CD2a/BTI-322 or MEDI-507. In another embodiment, an isolated nucleic acid molecule encodes an antibody that immunospecifically binds to a CD2 polypeptide, said antibody comprising a VH domain having the amino acid sequence of the VH domain of the monoclonal antibody produced by the cell line deposited with the ATCC® as Accession Number HB 11423. In another embodiment, an isolated nucleic acid molecule encodes an antibody that immunospecifically binds to a CD2 polypeptide, said antibody comprising a VH CDR1 having the amino acid sequence of the VH CDR1 listed in Table 2. In another embodiment, an isolated nucleic acid molecule encodes an antibody that immunospecifically binds to a polypeptide, said antibody comprising a VH CDR2 having the amino acid sequence of the VH CDR2 listed in Table 2. In yet another embodiment, an isolated nucleic acid molecule encodes an antibody that immunospecifically binds to a CD2 polypeptide, said antibody comprising a VH CDR3 having the amino acid sequence of the VH CDR3 listed in Table 2.

In one embodiment, an isolated nucleic acid molecule encodes an antibody that immunospecifically binds to a CD2 polypeptide, said antibody comprising a VL domain having the amino acid sequence of the VL domain of LO-CD2a/BTI-322 or MEDI-507. In another embodiment, an isolated nucleic acid molecule encodes an antibody that immunospecifically binds to a CD2 polypeptide, said antibody comprising a VL domain having the amino acid sequence of the VL domain of the monoclonal antibody produced by the cell line deposited with the ATCC® as Accession Number HB 1423. In another embodiment, an isolated nucleic acid molecule encodes an antibody that immunospecifically binds to a CD2 polypeptide, said antibody comprising a VL CDR1 having the amino acid sequence of the VL CDR1 listed in Table 2. In another embodiment, an isolated nucleic acid molecule encodes an antibody that immunospecifically bind to a CD2 polypeptide, said antibody comprising a VL CDR2 having the amino acid sequence of the VL CDR2 listed in Table 2. In yet another embodiment, an isolated nucleic acid molecule encodes an antibody that immunospecifically binds to a CD2 polypeptide, said antibody comprising a VL CDR3 having the amino acid sequence of the VL CDR3 listed in Table 2.

In another embodiment, an isolated nucleic acid molecule encodes an antibody that immunospecifically binds to a CD2 polypeptide, said antibody comprising a VH domain having the amino acid sequence of the VH domain of LO-CD2a/BTI-322 or MEDI-507 and a VL domain having the amino acid sequence of the VL domain of LO-CD2a/BTI-322 or MEDI-507. In another embodiment, an isolated nucleic acid molecule encodes an antibody that immunospecifically binds to a CD2 polypeptide, said antibody comprising a VH CDR1, a VL CDR1, a VH CDR2, a VL CDR2, a VH CDR3, a VL CDR3, or any combination thereof having an amino acid sequence listed in Table 2.

The present invention also provides antibodies that immunospecifically bind to a CD2 polypeptide, said antibodies comprising derivatives of the VH domains, VH CDRs, VL domains, or VL CDRs described herein that immunospecifically bind to a CD2 polypeptide. Standard techniques known to those of skill in the art can be used to introduce mutations in the nucleotide sequence encoding an antibody of the invention, including, for example, site-directed mutagenesis and PCR-mediated mutagenesis which results in amino acid substitutions. Preferably, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In a preferred embodiment, the derivatives have conservative amino acid substitutions are made at one or more predicted non-essential amino acid residues (i.e., amino acid residues which are not critical for the antibody to immunospecifically bind to a CD2 polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains ( e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

The present invention provides for antibodies that immunospecifically bind to a CD2 polypeptide, said antibodies comprising the amino acid sequence of LO-CD2a/BTI-322 or MEDI-507 with one or more amino acid residue substitutions in the variable light (VL) domain and/or variable heavy (VH) domain. The present invention also provides for antibodies that immunospecifically bind to a CD2 polypeptide, said antibodies comprising the amino acid sequence of LO-CD2a/BTI-322 or MEDI-507 with one or more amino acid residue substitutions in one or more VL CDRs and/or one or more VH CDRs. The antibody generated by introducing substitutions in the VH domain, VH CDRs, VL domain and/or VL CDRs of LO-CD2a/BTI-322 or MEDI-507 can be tested in vitro and in vivo, for example, for its ability to bind to a CD2 polypeptide, or for its ability to inhibit T-cell activation, or for its ability to inhibit T-cell proliferation, or for its ability to induce T-cell lysis, or for its ability to prevent, treat or ameliorate one or more symptoms associated with an autoimmune disorder or an inflammatory disorder.

In a specific embodiment, an antibody that immunospecifically binds to a CD2 polypeptide comprises a nucleotide sequence that hybridizes to the nucleotide sequence encoding the monoclonal antibody produced by the cell line deposited with the ATCC® as Accession Number HB 11423 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to a CD2 polypeptide comprises a nucleotide sequence that hybridizes to the nucleotide sequence encoding the MEDI-507 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In a specific embodiment, an antibody that immunospecifically binds to a CD2 polypeptide comprises an amino acid sequence of a VH domain or an amino acid sequence a VL domain encoded by a nucleotide sequence that hybridizes to the nucleotide sequence encoding the VH or VL domains of LO-CD2a/BTI-322 or MEDI-507 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, an antibody that immunospecifically binds to a CD2 polypeptide comprises an amino acid sequence of a VH CDR or an amino acid sequence of a VL CDR encoded by a nucleotide sequence that hybridizes to the nucleotide sequence encoding any one of the VH CDRs or VL CDRs listed in Table 2 under stringent conditions e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR or an amino acid sequence of a VL CDR encoded by a nucleotide sequence that hybridizes to the nucleotide sequence encoding any one of VH CDRs or VL CDRs of the monoclonal antibody produced by the cell line deposited with the ATCC® as Accession Number HB 11423 under stringent conditions e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR and an amino acid sequence of a VL CDR encoded by nucleotide sequences that hybridizes to the nucleotide sequences encoding any one of the VH CDRs and VL CDRs listed in Table 2 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In another embodiment, an antibody that immunospecifically binds to a CD2 polypeptide comprises an amino acid sequence of a VH CDR and an amino acid sequence of a VL CDR encoded by nucleotide sequences that hybridizes to the nucleotide sequences encoding the monoclonal antibody produced by the cell line deposited with the ATCC® as Accession Number HB 11423 under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art.

In a specific embodiment, an antibody that immunospecifically binds to a CD2 polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of the monoclonal antibody produced by the cell line deposited with the ATCC® as Accession Number HB 11423. In another embodiment, an antibody that immunospecifically binds to a CD2 polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of MEDI-507.

In another embodiment, an antibody that immunospecifically binds to a CD2 polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of MEDI-507. In another embodiment, an antibody that immunospecifically binds to a CD2 polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of the monoclonal antibody produced by the cell line deposited with the ATCC® as Accession Number HB 11423.

In another embodiment, an antibody that immunospecifically binds to a CD2 polypeptide comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VH CDRs listed in Table 2. In another embodiment, an antibody that immunospecifically binds to a CD2 polypeptide comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of one of the VH CDRs of the monoclonal antibody produced by the cell line deposited with the ATCC® as Accession Number HB 11423.

In another embodiment, an antibody that immunospecifically binds to a CD2 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of MEDI-507 In another embodiment, an antibody that immunospecifically binds to a CD2 polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of the monoclonal antibody produced by the cell line deposited with the ATCC® as Accession Number HB 11423.

In another embodiment, an antibody that immunospecifically binds to a CD2 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs listed in Table 2. In another embodiment, an antibody that immunospecifically binds to a CD2 polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any of the VL CDRs of the monoclonal antibody produced by the cell line deposited with the ATCC® as Accession Number HB 11423.

The present invention encompasses antibodies that compete with an antibody described herein for binding to a CD2 polypeptide. In a specific embodiment, the present invention encompasses antibodies that compete with LO-CD2a/BTI-322 or an antigen-binding fragment thereof for binding to the CD2 polypeptide. In a specific embodiment, the present invention encompasses antibodies that compete with LO-CD2b or an antigen-binding fragment for binding to a CD2 polypeptide. In a preferred embodiment, the present invention encompasses antibodies that compete with MEDI-507 or an antigen-binding fragment thereof for binding to the CD2 polypeptide.

The present invention also encompasses VH domains that compete with the VH domain of LO-CD2a/BTI-322 or MEDI-507 for binding to a CD2 polypeptide. The present invention also encompasses VL domains that compete with a VL domain of LO-CD2a/BTI-322 or MEDI-507 for binding to a CD2 polypeptide.

The present invention also encompasses VH CDRs that compete with a VH CDR listed in Table 2 for binding to a CD2 polypeptide, or a VH CDR of the monoclonal antibody produced by the cell line deposited with the ATCC as Accession Number HB 11423 for binding to a CD2 polypeptide. The present invention also encompasses VL CDRs that compete with a VL CDR listed in Table 2 for binding to a CD2 polypeptide, or a VL CDR of the monoclonal antibody produced by the cell line deposited with the ATCC as Accession Number HB 11423 for binding to a CD2 polypeptide.

The antibodies that immunospecifically bind to a CD2 polypeptide include derivatives that are modified, i.e, by the covalent attachment of any type of molecule to the antibody such that covalent attachment. For example, but not by way of limitation, the antibody derivatives include antibodies that have been modified, e.g., by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The present invention also provides antibodies that immunospecifically bind to a CD2 polypeptide, said antibodies comprising a framework region known to those of skill in the art. Preferably, the fragment region of an antibody of the invention is human. In a specific embodiment, an antibody that immunospecifically binds to a CD2 polypeptide comprises the framework region of MEDI-507.

The present invention also encompasses antibodies which immunospecifically bind to a CD2 polypeptide, said antibodies comprising the amino acid sequence of MEDI-507 with mutations (e.g., one or more amino acid substitutions) in the framework regions. In certain embodiments, antibodies which immunospecifically bind to a CD2 polypeptide comprise the amino acid sequence of MEDI-507 with one or more amino acid residue substitutions in the framework regions of the VH and/or VL domains.

The present invention also encompasses antibodies which immunospecifically bind to a CD2 polypeptide, said antibodies comprising the amino acid sequence of MEDI-507 with mutations (e.g, one or more amino acid residue substitutions) in the variable and framework regions.

The present invention also provides for fusion proteins comprising an antibody that immunospecifically binds to a CD2 polypeptide and a heterologous polypeptide. Preferably, the heterologous polypeptide that the antibody is fused to is useful for targeting the antibody to T-cells and/or NK cells.

### 5.2.3.1.1. Antibodies Having Increased Half-lives that Immunospecifically Bind to CD2 Polypeptides

The present invention provides for antibodies that immunospecifically bind to a CD2 polypeptide which have a extended half-life in vivo. In particular, the present invention provides antibodies that immunospecifically bind to a CD2 polypeptide which have a half-life in an animal, preferably a mammal and most preferably a human, of greater than 3 days, greater than 7 days, greater than 10 days, preferably greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (e.g., monoclonal antibodies, single chain antibodies and Fab fragments) in vivo, for example, inert polymer molecules such as high molecular weight polyethyleneglycol (PEG) can be attached to the antibodies with or without a multifunctional linker either through site-specific conjugation of the PEG to the—or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies can be tested for binding activity as well as for in vivo efficacy using methods well-known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (preferably a Fc or hinge-Fc domain fragment). See, e.g., International Publication No. WO 98/23289; International Publication No. WO 97/34631; and U.S. Pat. No.6,277,375, each of which is incorporated herein by reference in its entirety.

### 5.2.3.1.2. Antibody Conjugates

The present invention encompasses antibodies or antigen-binding fragments thereof that immunospecifically bind to a CD2 polypeptide recombinantly fused or chemically conjugated (including both covalently and non-covalently conjugations) to a heterologous polypeptide (or a fragment thereof, preferably at least 5, at least 10, at least 20, at least 30, at least 40, at least 50, at least 60, at least 70, at least 80, at least 90 or at least 100 contiguous amino acids of the polypeptide) to generate fusion proteins. The fusion does not necessarily need to be direct, but may occur through linker sequences. For example, antibodies may be used to target heterologous polypeptides to particular cell types (e.g., T-cells), either in vitro or in vivo, by fusing or conjugating the antibodies to antibodies specific for particular cell surface receptors such as, e.g., CD4 and CD8.

The present invention also encompasses antibodies or antigen-binding fragments thereof that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses antibodies or antigen-binding fragments thereof that immunospecifically bind to a CD2 polypeptide conjugated to an agent which has a potential therapeutic benefit. An antibody or an antigen-binding fragment thereof that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g, daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, an antibody or an antigen-binding fragment thereof that immunospecifically binds to a CD2 polypeptide may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, interferon-α (“IFN-α”), interferon-β (“IFN-β”), nerve growth factor (“NGF”), platelet derived growth factor (“PDGF”), tissue plasminogen activator (“TPA”), an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Iminunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., interleukin-1 (“IL-1”), IL-2, IL-6, IL-10, granulocyte macrophage colony stimulating factor (“GM-CSF”), and granulocyte colony stimulating factor (“G-CSF”)), or a growth factor (e.g., growth hormone (“GH”)).

Techniques for conjugating such therapeutic moieties to antibodies are well known, see, e.g., Arnon et al., “Monoclonal Antibodies For Immunotargeting Of Drugs In Cancer Therapy”, in Monoclonal Antibodies And Cancer Therapy, Reisfeld et al. (eds.), pp. 243-56 (Alan R. Liss, Inc. 1985); Hellstrom et al., “Antibodies For Drug Delivery”, in Controlled Drug Delivery (2nd Ed.), Robinson et al. (eds.), pp. 623-53 (Marcel Dekker, Inc. 1987); Thorpe, “Antibody Carriers Of Cytotoxic Agents In Cancer Therapy: A Review”, in Monoclonal Antibodies '84: Biological And Clinical Applications, Pinchera et al. (eds.), pp. 475-506 (1985); “Analysis, Results, And Future Prospective Of The Therapeutic Use Of Radiolabeled Antibody In Cancer Therapy”, in Monoclonal Antibodies For Cancer Detection And Therapy, Baldwin et al. (eds.), pp. 303-16 (Academic Press 1985); and

Thorpe et al., 1982, Immunol. Rev. 62:119-58.

An antibody or an antigen-binding fragment thereof that immunospecifically binds to a CD2 polypeptide can be conjugated to a second antibody to form an antibody heteroconjugate as described by Segal in U.S. Pat. No. 4,676,980, which is incorporated herein by reference in its entirety.

Antibodies or antigen-binding fragments thereof that immunospecifically bind to a CD2 polypeptide may be attached to solid supports, which are particularly useful for the purification of CD2⁺ immune cells such as T-cells. Such solid supports include, but are not limited to, glass, cellulose, polyacrylamide, nylon, polystyrene, polyvinyl chloride or polypropylene.

### 5.2.3.2. LFA-3 Polypeptides that Immununospecifically Bind to CD2 Polypeptides

The present invention encompasses LFA-3 peptides, polypeptides, derivatives and analogs thereof that immunospecifically bind to a CD2 polypeptide for use in the prevention, treatment or amelioration of one or more symptoms associated with an autoimmune or inflammatory disorder. Preferably, the soluble LFA-3 polypeptides that immunospecifically bind to a CD2 binding molecule comprise at least 5, preferably at least 10, at least 20, at least 30, at least 40, at least 50, at least 60, at least 70, at least 80, at least 90 or at least 100 contiguous amino acid residues of LFA-3. Soluble LFA-3 peptides, polypeptides, derivatives, and analogs thereof that immunospecifically bind to a CD2 binding molecule can be derived from any species.

The nucleotide and/or amino acid sequences of LFA-3 can be found in the literature or public databases, or the nucleic acid and/or amino acid sequences can be determined using cloning and sequencing techniques well-known to one of skill in the art. For example, the nucleotide and amino acid sequences of human LFA-3 can be found in the GenBank databases (see, e.g., Accession Nos. E12817 and CAA29622).

In a specific embodiment, a soluble LFA-3 polypeptide that immunospecifically binds to a CD2 polypeptide consists the extracellular domain of naturally occurring LFA-3 or amino acid residues 1 to 187 of SEQ ID NO:17. In another embodiment, a soluble LFA-3 polypeptide that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17).

In a specific embodiment, a soluble LFA-3 polypeptide that immunospecifically binds to a CD2 polypeptide inhibits or reduces the interaction between a CD2 polypeptide and LFA-3 by approximately 25%, 30%, 35%, 45%, 50%, 55%, 60%, 65%, 70%, 75%, 80%, 85%, 90%, 95%, or 98% in an in vivo or in vitro assay described herein or well-known to one of skill in the art. In an alternative embodiment, a soluble LFA-3 polypeptide that immunospecifically binds to a CD2 polypeptide does not inhibit the interaction between a CD2 polypeptide and LFA-3 in an in vivo or in vitro assay described herein or well-known to one of skill in the art. In another embodiment, a soluble LFA-3 polypeptide that immunospecifically binds to a CD2 polypeptide inhibits the interaction between a CD2 polypeptide and LFA-3 by less than 20%, less than 15%, less than 10%, or less than 5%.

In a specific embodiment, soluble LFA-3 polypeptides that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art. In another embodiment, soluble LFA-3 polypeptides that immunospecifically bind to a CD2 polypeptide inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art. In another embodiment, soluble LFA-3 polypeptides that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or well-known to one of skill in the art.

In another embodiment, a soluble LFA-3 polypeptide that immunospecifically binds to a CD2 polypeptide does not induce or reduces cytokine expression and/or release in an in vivo or in vitro assay described herein or well-known to one of skill in the art. In a specific embodiment, soluble LFA-3 polypeptide that immunospecifically binds to a CD2 polypeptide does not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, a soluble LFA-3 polypeptide that immunospecifically binds to a CD2 polypeptide induces cytokine expression and/or release in an in vitro or in vivo assay described herein or well-known to one of skill in the art. In a specific embodiment, a soluble LFA-3 polypeptide that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g., ELISA.

In another embodiment, a soluble LFA-3 polypeptide that immunospecifically binds to a CD2 polypeptide induces T-cell anergy in an in vivo or in vitro assay described herein or known to one of skill in the art. In an alternative embodiment, a soluble LFA-3 polypeptide that immunospecifically binds to a CD2 polypeptide does not induce T-cell anergy in an in vivo or in vitro assay described herein or known to one of skill in the art. In another embodiment, a soluble LFA-3 polypeptide that immuno specifically binds to a CD2 polypeptide elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or known to one of skill in the art.

In a specific embodiment, soluble LFA-3 polypeptides that immuno specifically bind to a CD2 polypeptide mediate depletion of peripheral blood T-cells by inducing cytolysis of T-cells. In another preferred embodiment, soluble LFA-3 polypeptides that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or known to one of skill in the art.

The present invention provides for soluble LFA-3 polypeptides that immunospecifically bind to a CD2 polypeptide which have a extended half-life in vivo. In particular, the present invention provides soluble LFA-3 polypeptides that immunospecifically bind to a CD2 polypeptide which have a half-life in an animal, preferably a mammal and most preferably a human, of greater than 3 days, greater than 7 days, greater than 10 days, preferably greater than 15 days, greater than 25 days, greater than days, greater than 35 days, greater than 40 days, greater than 45 days, greater than 2 months, greater than 3 months, greater than 4 months, or greater than 5 months.

To prolong the serum circulation of soluble LFA-3 polypeptides that immuno specifically bind to a CD2 polypeptide in vivo, for example, inert polymer molecules such as high molecular weight polyethyleneglycol (PEG) can be attached to the antibodies with or without a multifuinctional linker either through site-specific conjugation of the PEG to the—or C-terminus of the soluble LFA-3 polypeptides or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the soluble LFA-3 polypeptides. Unreacted PEG can be separated from LFA-3 polypeptide-PEG conjugates by size-exclusion or by ion-exchange chromatography. PEG-derivatized LFA-3 polypeptides can be tested for binding activity as well as for in vivo efficacy using methods well-known to those of skill in the art, for example, by immunoassays described herein.

### 5.2.3.2.1. LFA-3 Conjugates

The present invention also encompasses soluble LFA-3 peptides and polypeptides that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the soluble LFA-3 polypeptide. Other peptide tags useful for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses soluble LFA-3 peptides and polypeptides that immunospecifically bind to a CD2 polypeptide conjugated to a therapeutic agent. A soluble LFA-3 polypeptide that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a soluble LFA-3 polypeptide that immunospecifically binds to a CD2 polypeptide may be conjugated to a a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, IFN-α, IFN-β, nerve growth factor (“NGF”), platelet derived growth factor (“PDGF”), tissue plasminogen activator (“TPA”), an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., IL-1, IL-2, IL-6, IL-10, GM-CSF, and G-CSF), or a growth factor (e.g., GH).

### 5.2.3.3. Fusion Proteins that Immunospecifically Bind to CD2 Polypeptides

The present invention provides fusion proteins that immunospecifically bind to a CD2 polypeptide and modulate an activity or function of lymphocytes, preferably peripheral blood T-cells for use in preventing, treating or ameliorating one or more symptoms associated with an autoimmune disorder or an inflammatory disorder. Preferably, such fusion proteins directly or indirectly mediate depletion of lymphocytes, in particular peripheral blood T-cells. In particular, the present invention provides fusion proteins that immunospecifically bind to a CD2 polypeptide expressed by an immune cell such as a T-cell or NK cell and mediate depletion of lymphocytes, in particular peripheral blood T-cells.

In a specific embodiment, a fusion protein that immunospecifically binds to a CD2 polypeptide inhibits or reduces the interaction between a CD2 polypeptide and LFA-3 by approximately 25%, 30%, 35%, 45%, 50%, 55%, 60%, 65%, 70%, 75%, 80%, 85%, 95%, or 98% in an in vivo or in vitro assay described herein or well-known to one of skill in the art. In an alternative embodiment, a fusion protein that immunospecifically binds to a CD2 polypeptide does not inhibit the interaction between a CD2 polypeptide and LFA-3 in an in vivo or in vitro assay described herein or well-known to one of skill in the art. In another embodiment, a fusion protein that immunospecifically binds to a CD2 polypeptide inhibits the interaction between a CD2 polypeptide and LFA-3 by less than 20%, less than 15%, less than 10%, or less than 5%.

In another embodiment, a fusion protein that immunospecifically binds to a CD2 polypeptide does not induce or reduces cytokine expression and/or release in an in vivo or in vitro assay described herein or well-known to one of skill in the art. In a specific embodiment, fusion protein that immunospecifically binds to a CD2 polypeptide does not induce an increase in the concentration cytokines such as, e.g., IFN-γ, IL-2, IL-4, IL-6, IL-9, IL-12, and IL-15 in the serum of a subject administered a CD2 binding molecule. In an alternative embodiment, a fusion protein that immunospecifically binds to a CD2 polypeptide induces cytokine expression and/or release in an in vitro or in vivo assay described herein or well-known to one of skill in the art. In a specific embodiment, a fusion protein that immunospecifically binds to a CD2 polypeptide induces an increase in the concentration of cytokines such as, e.g., IFN-γ, IL-2, IL4, IL-6, IL-7, IL-9, IL-10, and TNF-α in the serum of a subject administered a CD2 binding molecule. Serum concentrations of a cytokine can be measured by any technique well-known to one of skill in the art such as, e.g., ELISA.

In another embodiment, a fusion protein that immunospecifically binds to a CD2 polypeptide induces T-cell anergy in an in vivo or in vitro assay described herein or well-known to one of skill in the art. In an alternative embodiment, a fusion protein that immunospecifically binds to a CD2 polypeptide does not induce T-cell anergy in an in vivo or in vitro assay described herein or well-known to one of skill in the art. In another embodiment, a fusion protein that immunospecifically binds to a CD2 polypeptide elicits a state of antigen-specific unresponsiveness or hyporesponsiveness for at least 30 minutes, at least 1 hour, at least 2 hours, at least 6 hours, at least 12 hours, at least 24 hours, at least 2 days, at least 5 days, at least 7 days, at least 10 days or more in an in vitro assay described herein or well-known to one of skill in the art.

In a specific embodiment, fusion proteins that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assays described herein or well-known to one of skill in the art. In a preferred, fusion proteins that immunospecifically bind to a CD2 polypeptide mediate depletion of peripheral blood T-cells by inducing cytolysis of T-cells. In another preferred embodiment, fusion proteins that immunospecifically bind to a CD2 polypeptide mediate depletion of peripheral blood T-cells by inhibiting T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inducing cytolysis of peripheral blood T-cells in an in vivo or in vitro assay described herein or well-known to one of skill in the art.

In another embodiment, fusion proteins that immunospecifically bind to a CD2 polypeptide inhibit T-cell activation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% and inhibit T-cell proliferation by at least 25%, at least 30%, at least 35%, at least 40%, at least 50%, at least 55%, at least 60%, at least 65%, at least 70%, at least 75%, at least 80%, at least 85%, at least 90%, at least 95%, or at least 98% in an in vivo or in vitro assay described herein or known to one of skill in the art. In another embodiment, a fusion protein that immunospecifically binds to a CD2 polypeptide binds to an FcR expressed by an immune cell such as an NK cell, a monocyte, and macrophage. In a preferred embodiment, a fusion protein that immunospecifically binds to a CD2 polypepitde binds to an FcγRIII expressed by an immune cell such as an NK cell, a monocyte, and a macrophage.

In one embodiment, a fusion protein that immunospecifically binds to a CD2 polypeptide comprises a bioactive molecule fused to the Fc domain of an immunoglobulin molecule or a fragment thereof. In another embodiment, a fusion protein that immunospecifically binds to a CD2 polypeptide comprises a bioactive molecule fused to the CH2 and /or CH3 region of the Fc domain of an immunoglobulin molecule. In yet another embodiment, a fusion protein that immunospecifically binds to a CD2 polypeptide comprises a bioactive molecule fused to the CH2, CH3, and hinge regions of the Fc domain of an immunoglobulin molecule. In accordance with these embodiments, the bioactive molecule immunospecifically binds to a CD2 polypeptide. Bioactive molecules that immunospecifically bind to a CD2 polypeptide include, but are not limited to, peptides, polypeptides, small molecules, mimetic agents, synthetic drugs, inorganic molecules, and organic molecules. Preferably, a bioactive molecule that immunospecifically binds to a CD2 polypeptide is a polypeptide comprising at least 5, preferably at least 10, at least 20, at least 30, at least 40, at least 50, at least 60, at least 70, at least 80, at least 90 or at least 100 contiguous amino acid residues, and is heterologous to the amino acid sequence of the Fc domain of an immunoglobulin molecule or a fragment thereof.

In a specific embodiment, a fusion protein that immunospecifically binds to a CD2 polypeptide comprises LFA-3 or a fragment thereof which immunospecifically binds to a CD2 polypeptide fused to the Fc domain of an immunoglobulin molecule or a fragment thereof. In another embodiment, a fusion protein that immunospecifically binds to a CD2 polypeptide comprises LFA-3 or a fragment thereof which immunospecifically binds to a CD2 polypeptide fused to the CH2 and/or CH3 region of the Fc domain of an immunoglobulin molecule. In another embodiment, a fusion protein that immunospecifically binds to a CD2 polypeptide comprises LFA-3 or a fragment thereof which immunospecifically binds to a CD2 polypeptide fused to the CH2, CH3, and hinge regions of the Fc domain of an immunoglobulin molecule.

In another embodiment, a fusion protein that immunospecifically binds to a CD2 polypeptide comprises an extracellular domain of LFA-3 (e.g., amino acid residues 1 to 187 of SEQ ID NO:17) fused to the Fc domain of an immunoglobulin molecule or a fragment thereof. In another embodiment, a fusion protein that immunospecifically binds to a CD2 polypeptide comprises an extracellular domain of LFA-3 (e.g., amino acid residues 1 to 187 of SEQ ID NO:17) fused to the CH2 and/or CH3 region of the Fc domain of an immunoglobulin molecule. In another embodiment, a fusion protein that immunospecifically binds to a CD2 polypeptide comprises an extracellular domain of LFA-3 (e.g., amino acid residues 1 to 187 of SEQ ID NO:17) fused to the CH2, CH3, and hinge regions of the Fc domain of an immunoglobulin molecule.

In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the Fc domain of an immunoglobulin molecule or a fragment thereof. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the CH2 and/or CH3 region of the Fc domain of an immunoglobulin molecule. In another embodiment, a fusion protein that immunospecifically binds to a CD2 polypeptide comprises a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the CH2, CH3, and binge regions of the Fc domain of an immunoglobulin molecule.

In a specific embodiment, a CD2 binding molecule is LFA-3TIP (Biogen, Inc., Cambridge, Mass.). In an alterative embodiment, a CD2 binding molecule is not LFA-3TIP.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the Fc domain of an immunoglobulin molecule or a fragment thereof. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2 and/or CH3 region of the Fc domain of an immunoglobulin molecule. In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of LFA-3 or a fragment thereof fused to the CH2, CH3, and hinge regions of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:17) fused to the Fc domain of an immunoglobulin molecule or a fragment thereof. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187of SEQ ID NO:17) fused to the CH2 and/or CH3 region of the Fc domain of an immunoglobulin molecule. In another embodiment, a fusion protein that immunospecifically binds to a CD2 polypeptide comprise a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of an extracellular domain of LFA-3 (e.g., amino acid residues 1 to 187 of SEQ ID NO:17) fused to the CH2, CH3, and hinge regions of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the Fc domain of an immunoglobulin molecule or a fragment thereof.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the CH2 and/or CH3 region of the Fc domain of an immunoglobulin molecule.

In another embodiment, a fusion protein that immunospecifically binds to a CD2 polypeptide comprises a polypeptide having an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of a fragment of an extracellular domain of LFA-3 (e.g, amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) fused to the CH2, CH3, and hinge regions of the Fc domain of an immunoglobulin molecule.

The present invention provides fusion proteins that immunospecifically bind to a CD2 polypeptide comprising the Fc domain of an immunoglobulin molecule or a fragment thereof fused to a polypeptide encoded by a nucleic acid molecule that hybridizes to the nucleotide sequence encoding LFA-3 or a fragment thereof.

In a specific embodiment, a fusion protein that immunospecifically binds to a CD2 polypeptide comprises the Fc domain of an immunoglobulin molecule or a fragment thereof fused to a polypeptide encoded by a nucleic acid molecule that hybridizes to the nucleotide sequence encoding LFA-3 or a fragment thereof under stringent conditions, e.g, hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In another embodiment, a fusion protein that immunospecifically binds to a CD2 polypeptide comprises the Fc domain of an immunoglobulin molecule or a fragment thereof fused to a polypeptide encoded by a nucleic acid molecule that hybridizes to the nucleotide sequence encoding an extracellular domain of LFA-3 (e.g., amino acid residues 1 to 187of SEQ ID NO:17) under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. 1, Green Publishing Associates, Inc. and John Wiley & Sons, Inc., New York at pages 6.3.1-6.3.6 and 2.10.3).

In yet another embodiment, a fusion protein that immunospecifically binds to a CD2 polypeptide comprises the Fc domain of an immunoglobulin molecule or a fragment thereof fused to a polypeptide encoded by a nucleic acid molecule that hybridizes to the nucleotide sequence encoding the amino acid sequence of a fragment of an extracellular domain of LFA-3 (e.g., amino acid residues 1 to 92, amino acid residues 1 to 85, amino acid residues 1 to 80, amino acid residues 1 to 75, amino acid residues 1 to 70, amino acid residues 1 to 65, or amino acid residues 1 to 60 SEQ ID NO:17) under stringent conditions, e.g., hybridization to filter-bound DNA in 6×sodium chloride/sodium citrate (SSC) at about 45° C. followed by one or more washes in 0.2×SSC/0.1% SDS at about 50-65° C., under highly stringent conditions, e.g., hybridization to filter-bound nucleic acid in 6×SSC at about 45° C. followed by one or more washes in 0.1×SSC/0.2% SDS at about 68° C., or under other stringent hybridization conditions which are known to those of skill in the art (see, for example, Ausubel, F. M. et al., eds., 1989, Current Protocols in Molecular Biology, Vol. I, Green Publishing Associates, Inc. and John Wiley & Sons, Inc., New York at pages 6.3.1-6.3.6 and 2.10.3).

### 10 5.2.3.3.1. Fusion Protein Conjugates

The present invention also encompasses fusion proteins that immunospecifically bind to a CD2 polypeptide fused to marker sequences, such as a peptide to facilitate purification. In preferred embodiments, the marker amino acid sequence is a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags usefil for purification include, but are not limited to, the hemagglutinin “HA” tag, which corresponds to an epitope derived from the influenza hemagglutinin protein (Wilson et al., 1984, Cell 37:767) and the “flag” tag.

The present invention further encompasses fusion proteins that immunospecifically bind to a CD2 polypeptide conjugated to a therapeutic agent. A fusion protein that immunospecifically binds to a CD2 polypeptide may be conjugated to a therapeutic moiety such as a cytotoxin, e.g., a cytostatic or cytocidal agent, an agent which has a potential therapeutic benefit, or a radioactive metal ion, e.g., alpha-emitters. A cytotoxin or cytotoxic agent includes any agent that is detrimental to cells. Examples of a cytotoxin or cytotoxic agent include, but are not limited to, paclitaxol, cytochalasin B, gramicidin D, ethidium bromide, emetine, mitomycin, etoposide, tenoposide, vincristine, vinblastine, colchicin, doxorubicin, daunorubicin, dihydroxy anthracin dione, mitoxantrone, mithramycin, actinomycin D, 1-dehydrotestosterone, glucocorticoids, procaine, tetracaine, lidocaine, propranolol, and puromycin and analogs or homologs thereof. Agents which have a potential therapeutic benefit include, but are not limited to, antimetabolites (e.g., methotrexate, 6-mercaptopurine, 6-thioguanine, cytarabine, 5-fluorouracil decarbazine), alkylating agents (e.g., mechlorethamine, thioepa chlorambucil, melphalan, carmustine (BSNU) and lomustine (CCNU), cyclothosphamide, busulfan, dibromomannitol, streptozotocin, mitomycin C, and cisdichlorodiamine platinum (II) (DDP) cisplatin), anthracyclines (e.g., daunorubicin (formerly daunomycin) and doxorubicin), antibiotics (e.g., dactinomycin (formerly actinomycin), bleomycin, mithramycin, and anthramycin (AMC)), and anti-mitotic agents (e.g., vincristine and vinblastine).

Further, a fusion protein that immunospecifically binds to a CD2 polypeptide may be conjugated to a therapeutic agent or drug moiety that modifies a given biological response. Agents which have a potential therapeutic benefit or drug moieties are not to be construed as limited to classical chemical therapeutic agents. For example, the drug moiety may be a protein or polypeptide possessing a desired biological activity. Such proteins may include, for example, a toxin such as abrin, ricin A, pseudomonas exotoxin, or diphtheria toxin; a protein such as tumor necrosis factor, IFN-α, IFN-β, NGF, PDGF, TPA, an apoptotic agent, e.g., TNF-α, TNF-β, AIM I (see, International Publication No. WO 97/33899), AIM II (see, International Publication No. WO 97/34911), Fas Ligand (Takahashi et al., 1994, J. Immunol., 6:1567-1574), and VEGF (see, International Publication No. WO 99/23105), a thrombotic agent or an anti-angiogenic agent, e.g., angiostatin or endostatin; or, a biological response modifier such as, for example, a lymphokine (e.g., IL-1, IL-2, IL-6, IL-10, GM-CSF, and G-CSF), or a growth factor (e.g., GH).

5.2.4. Anti-angiogenic Agents

Any anti-angiogenic agents well-known to one of skill in the art can be used in the compositions and methods of the invention. Non-limiting examples include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that reduce or inhibit or neutralizes the angiogenesis. In particular, examples of anti-angiogenic agents, include, but are not limited to, endostatin, angiostatin, apomigren, anti-angiogenic antithrombin III, the 29 kDa N-terminal and a 40 kDa C-terminal proteolytic fragments of fibronectin, a uPA receptor antagonist, the 16 kDa proteolytic fragment of prolactin, the 7.8 kDa proteolytic fragment of platelet factor-4, the anti-angiogenic 24 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, RGD and NGR containing peptides, the small anti-angiogenic peptides of laminin, fibronectin, procollagen and EGF, integrin α_{V}β₃ antagonists (e.g., anti-integrin α_{V}β₃ antibodies), acid fibroblast growth factor (aFGF) antagonists, basic fibroblast growth factor (bFGF) antagonists, vascular endothelial growth factor (VEGF) antagonists, and VEGF receptor (VEGFR) antagonists (e.g., anti-VEGFR antibodies).

In a specific embodiment of the invention, an anti-angiogenic agent is endostatin. Naturally occurring endostatin consists of the C-terminal˜180 amino acids of collagen XVIII (cDNAs encoding two splice forms of collagen XVIII have GenBank Accession Nos. AF18081 and AF18082). In another embodiment of the invention, an anti-angiogenic agent is a plasminogen fragment (the coding sequence for plasminogen can be found in GenBank Accession Nos. NM_{—}000301 and A33096). Angiostatin peptides naturally include the four kringle domains of plasminogen, kringle 1 through kringle 4. It has been demonstrated that recombinant kringle 1, 2 and 3 possess the anti-angiogenic properties of the native peptide, whereas kringle 4 has no such activity (Cao et al., 1996, J. Biol. Chem. 271:29461-29467). Accordingly, the angiostatin peptides comprises at least one and preferably more than one kringle domain selected from the group consisting of kringle 1, kringle 2 and kringle 3. In a specific embodiment, the anti-angiogenic peptide is the 40 kDa isoform of the human angiostatin molecule, the 42 kDa isoform of the human angiostatin molecule, the 45 kDa isoform of the human angiostatin molecule, or a combination thereof. In another embodiment, an anti-angiogenic agent is the kringle 5 domain of plasminogen, which is a more potent inhibitor of angiogenesis than angiostatin (angiostatin comprises kringle domains 1-4). In another embodiment of the invention, an anti-angiogenic agent is antithrombin III. Antithrombin III, which is referred to hereinafter as antithrombin, comprises a heparin binding domain that tethers the protein to the vasculature walls, and an active site loop which interacts with thrombin. When antithrombin is tethered to heparin, the protein elicits a conformational change that allows the active loop to interact with thrombin, resulting in the proteolytic cleavage of said loop by thrombin. The proteolytic cleavage event results in another change of conformation of antithrombin, which (i) alters the interaction interface between thrombin and antithrombin and (ii) releases the complex from heparin (Carrell, 1999, Science 285:1861-1862, and references therein). O''Reilly et al. (1999, Science 285:1926-1928) have discovered that the cleaved antithrombin has potent anti-angiogenic activity. Accordingly, in one embodiment, an anti-angiogenic agent is the anti-angiogenic form of antithrombin. In another embodiment of the invention, an anti-angiogenic agent is the 40 kDa and/or 29 kDa proteolytic fragment of fibronectin.

In another embodiment of the invention, anti-angiogenic agent is a urokinase plasminogen activator (uPA) receptor antagonist. In one mode of the embodiment, the antagonist is a dominant negative mutant of uPA (see, e.g., Crowley et al., 1993, Proc. Natl. Acad. Sci. USA 90:5021-5025). In another mode of the embodiment, the antagonist is a peptide antagonist or a fusion protein thereof (Goodson et al., 1994, Proc. Natl. Acad. Sci. USA 91:7129-7133). In yet another mode of the embodiment, the antagonist is a dominant negative soluble uPA receptor (Min et al., 1996, Cancer Res. 56:2428-2433). In another embodiment of the invention, a therapeutic molecule of the invention is the 16 kDa N-terminal fragment of prolactin, comprising approximately 120 amino acids, or a biologically active fragment thereof (the coding sequence for prolactin can be found in GenBank Accession No. NM_{—}000948). In another embodiment of the invention, an anti-angiogenic agent is the 7.8 kDa platelet factor-4 fragment. In another embodiment of the invention, a therapeutic molecule of the invention is a small peptide corresponding to the anti-angiogenic 13 amino acid fragment of platelet factor-4, the anti-angiogenic factor designated 13.40, the anti-angiogenic 22 amino acid peptide fragment of thrombospondin I, the anti-angiogenic 20 amino acid peptide fragment of SPARC, the small anti-angiogenic peptides of laminin, fibronectin, procollagen, or EGF, or small peptide antagonists of integrin α_{V}β₃ or the VEGF receptor. In another embodiment, the small peptide comprises an RGD or NGR motif. In certain embodiments, an anti-angiogenic agent is a TNF-α antagonist. In other embodiments, an anti-angiogenic agent is not a TNF-α antagonist.

5.2.5. TNF-α Antagonists

Any TNF-α antagonist well-known to one of skill in the art can be used in the compositions and methods of the invention. Non-limiting examples of TNF-α antagonists include proteins, polypeptides, peptides, fusion proteins, antibodies (e.g., human, humanized, chimeric, monoclonal, polyclonal, Fvs, ScFvs, Fab fragments, F(ab)₂ fragments, and antigen-binding fragments thereof) such as antibodies that immunospecifically bind to TNF-α, nucleic acid molecules (e.g., antisense molecules or triple helices), organic molecules, inorganic molecules, and small molecules that blocks, reduces, inhibits or neutralizes the function, activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, at least 95% or at least 99% relative to a control such as phosphate buffered saline (PBS).

Examples of antibodies that immunospecifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that immunospecifically bind to TNF-α disclosed in the following U.S. Patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Inh (Seckinger et al, 1990, Proc. Natl. Acad. Sci. USA 87:5188-5192).

In one embodiment, a TNF-α antagonist used in the compositions and methods of the invention is a soluble TNF-α receptor. In a specific embodiment, a TNF-α antagonist used in the compositions and methods of the invention is etanercept (ENBREL™; Immunex) or a fragment, derivative or analog thereof. In another embodiment, a TNF-α antagonist used in the compositions and methods of the invention is an antibody that immunospecifically binds to TNF-α. In a specific embodiment, a TNF-α antagonist used in the compositions and methods of the invention is infliximab (REMICADE™; Centacor) a derivative, analog or antigen-binding fragment thereof.

Other TNF-α antagonists encompassed by the invention include, but are not limited to, IL-10, which is known to block TNF-α production via interferon y-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (innmunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Nucleic acid molecules encoding proteins, polypeptides, or peptides with TNF-α antagonist activity or proteins, polypeptides, or peptides with TNF-α antagonist activity can be administered to a subject with an inflammatory or autoimmune disease in accordance with the methods of the invention. Further, nucleic acid molecules encoding derivatives, analogs, fragments or variants of proteins, polypeptides, or peptides with TNF-α antagonist activity, or derivatives, analogs, fragments or variants of proteins, polypeptides, or peptides with TNF-α antagonist activity can be administered to a subject with an inflammatory or autoimmune disease in accordance with the methods of the invention. Preferably, such derivatives, analogs, variants and fragments retain the TNF-α antagonist activity of the full-length wild-type protein, polypeptide, or peptide.

Proteins, polypeptides, or peptides that can be used as TNF-α antagonists can be produced by any technique well-known in the art or described herein. Proteins, polypeptides or peptides with TNF-α antagonist activity can be engineered so as to increase the in vivo half-life of such proteins, polypeptides, or peptides utilizing techniques well-known in the art or described herein. Preferably, agents that are commercially available and known to function as TNF-α antagonists are used in the compositions and methods of the invention. The TNF-α antagonist activity of an agent can be determined in vitro and/or in vivo by any technique well-known to one skilled in the art.

5.2.6. Anti-Inflammatory Agents

Anti-inflammatory agents have exhibited success in treatment of inflammatory and autoimmune disorders and are now a common and a standard treatment for such disorders. Any anti-inflammatory agent well-known to one of skill in the art can be used in the compositions and methods of the invention. Non-limiting examples of anti-inflammatory agents include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (e.g., COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

5.3. Prophylactic and Therapeutic Uses of Combination Therapy

The present invention provides methods of preventing, treating, managing or ameliorating one or more symptoms associated with an autoimmune or inflammatory disorder in a subject, said methods comprising administering to said subject one or more integrin α_{V}β₃ antagonists and one or more prophylactic or therapeutic agents other than integrin α_{V}β₃ antagonists, which prophylactic or therapeutic agents are currently being used, have been used or are known to be useful in the prevention, treatment or amelioration of one or more symptoms associated with an autoimmune disorder or inflammatory disorder. Section 5.2 provides non-limiting examples of the prophylactic or therapeutic agents which can be used in conjunction with integrin α_{V}β₃ antagonists for the prevention, treatment, management or amelioration of one or more symptoms associated with an autoimmune disorder or inflammatory disorder.

In a specific embodiment, the present invention provides a method for preventing, treating, managing, or ameliorating one or more symptoms associated with an autoimmune or inflammatory disorder in a subject, said method comprising administering to said subject one or more integrin α_{V}β₃ antagonists and one or more prophylactic or therapeutic agents other than integrin α_{V}β₃ antagonists, wherein at least one of the integrin α_{V}β₃ antagonists is an antibody or fragment thereof that immunospecifically binds to integrin α_{V}β₃. In a preferred embodiment, the present invention provides a method for preventing, treating, managing, or ameliorating one or more symptoms associated with an autoimmune or inflammatory disorder in a subject, said method comprising administering to said subject one or more integrin α_{V}β₃ antagonists and one or more prophylactic or therapeutic agents other than integrin α_{V}β₃ antagonists, wherein at least one of the integrin α_{V}β₃ antagonists is the humanized monoclonal MEDI-522 (known under the trade name VITAXIN™) or an antigen-binding fragment thereof.

Examples of autoimmune disorders which can be prevented, treated or managed in accordance with the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behcet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immunemediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, and Wegener's granulomatosis. Examples of inflammatory disorders which can be prevented, treated or managed in accordance with the methods of the invention include, but are not limited to, asthma, encephilitis, inflammatory bowel disease, chronic obstructive pulmonary disease (COPD), allergic disorders, septic shock, pulmonary fibrosis, undifferentitated spondyloarthropathy, undifferentiated arthropathy, arthritis, inflammatory osteolysis, and chronic inflammation resulting from chronic viral or bacteria infections.

The present invention provides methods of preventing, treating, managing or ameliorating one or more symptoms associated with an autoimmune or inflammatory disorder in a subject, said methods comprising administering to said subject one or more integrin α_{V}β₃ antagonists and one or more immunomodulatory agents. Preferably, the immunomodulatory agents are not administered to a subject with an autoimmune or inflammatory disorder whose absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³. Thus, in a preferred embodiment, prior to or subsequent to the administration of one or more dosages of one or more immunomodulatory agents to a subject with an autoimmune or inflammatory disorder, the absolute lymphocyte count of said subject is determined by techniques well-known to one of skill in the art, including, e.g., flow cytometry or trypan blue counts. Section 5.2 provides non-limiting examples of immunomodulatory agents which can be used in accordance with the methods of the invention.

In a specific embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more immunomodulatory agents. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more immunomodulatory agents, wherein at least one of the integrin α_{V}β₃ antagonists is an antibody or fragment thereof that immunospecifically binds to integrin α_{V}β₃ In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more immunomodulatory agents, wherein at least one of the integrin α_{V}β₃ antagonists is VITAXIN™ or an antigen-binding fragment thereof. In another preferred embodiment, the present invention provides a method of preventing, treating or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of one or more immunomodulatory agents.

In a specific embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of methotrexate or cyclosporin. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of VITAXIN™ and a prophylactically or therapeutically effective amount of methotrexate or cyclosporin. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists, a prophylactically or therapeutically effective amount of methotrexate, and a prophylactically or therapeutically effective amount of cyclosporin.

The present invention provides methods for preventing, treating, managing or ameliorating one or more symptoms associated with an autoimmune or inflammatory disorder in a subject, said methods comprising administering to said subject one or more integrin α_{V}β₃ antagonists and one or more CD2 binding molecules (e.g., peptides, polypeptides, proteins, antibodies (MEDI-507), and fusion proteins that immunospecifically bind to a CD2 polypeptide and mediate, directly or indirectly, the depletion of peripheral blood lymphocytes). Preferably, CD2 binding molecules are not administered to a subject with an autoimmune or inflammatory disorder whose absolute lymphocyte count is less than 500 cells/mm³, less than 550 cells/mm³, less than 600 cells/mm³, less than 650 cells/mm³, less than 700 cells/mm³, less than 750 cells/mm³, less than 800 cells/mm³, less than 850 cells/mm³ or less than 900 cells/mm³. Thus, in a preferred embodiment, prior to or subsequent to the administration of one or more dosages of one or more CD2 binding molecules to a subject with an autoimmune or inflammatory disorder, the absolute lymphocyte count of said subject is determined by techniques well-known to one of skill in the art, including, e.g., flow cytometry or trypan blue counts.

In a specific embodiment, the percentage of CD2 polypeptides bound by CD2 binding molecules is assessed after the administration of a first dose of one or more CD2 binding molecules to a subject with an autoimmune or inflammatory disorder and prior to the administration of one or more subsequent doses of one or more CD2 binding molecules. In another embodiment, the percentage of CD2 polypeptides bound by CD2 binding molecules is assessed regularly (e.g., every week, every two weeks, every three weeks, every 4 weeks, every 5 weeks, every 8 weeks, or every 12 weeks) following the administration one or more doses of CD2 binding molecules to a subject with an autoimmune or inflammatory disorder. Preferably, a subject with an autoimmune or inflammatory disorder is administered a subsequent dosage of one or more CD2 binding molecules if the percentage of CD2 polypeptides bound by CD2 binding molecules is less than 80%, preferably less than 75%, less than 70%, less than 65%, less than 50%, less than 45%, less than 40%, less than 35%, less than 30%, less than 25%, or less than 20%. The percentage of CD2 polypeptides bound to CD2 binding molecules can be assessed utilizing techniques well-known to one of skill in the art or described herein.

In a specific embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding molecules. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is an antibody or fragment thereof that immunospecifically binds to integrin α_{V}β₃. In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is VITAXIN™ or an antigen-binding fragment thereof. In another preferred embodiment, the present invention provides a method of preventing, treating or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of one or more CD2 binding molecules.

In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding molecules, wherein at least one of the CD2 binding molecules is soluble LFA-3 polypeptide or LFA3TIP. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more immunomodulatory agents, wherein at least one of the CD2 binding molecules is an antibody or fragment thereof that immunospecifically binds to a CD2 polypeptide. In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more immunomodulatory agents, wherein at least one of CD2 binding molecules is MEDI-507 or an antigen-binding fragment thereof.

In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding molecules, wherein at least one of the integrin α_{V}β₃ antagonists is an antibody or fragment thereof that immunospeciflcally binds to integrin α_{V}β₃ and wherein at least one of the CD2 binding molecules is a soluble LFA-3 polypeptide or LFA3TIP.

In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more CD2 binding, wherein at least one of the integrin α_{V}β₃ antagonists is VITAXIN™ or an antigen-binding fragment thereof and wherein at least one of the CD2 binding molecules or antigen-binding fragment thereof. In another preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of one or more CD2 binding, wherein at least one of the CD2 binding molecules or antigen-binding fragment thereof. In yet another preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of MEDI-507 or antigen-binding fragment.

The present invention provides methods of preventing, treating, managing or ameliorating one or more symptoms associated with an inflammatory disorder or an autoimmune disorder associated with inflammation in a subject, said methods comprising administering to said subject one or more integrin α_{V}β₃ antagonists and one or more TNF-α antagonists. Section 5.2 provides non-limiting examples of TNF-α antagonists which can be used in accordance with the methods of the invention.

In a specific embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is an antibody or fragment thereof that immunospecifically binds to integrin α_{V}β₃.

In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is VITAXIN™ or an antigen-binding fragment thereof. In another preferred embodiment, the present invention provides a method of preventing, treating or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of one or more TNF-α antagonists.

In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists, wherein at least one of the TNF-α antagonists is a soluble TNF-α receptor such as etanercept (ENBREL™; Immunex) or a fragment, derivative or analog thereof, or an antibody that immunospecifically binds to TNF-α such as infliximab (REMICADE™; Centacor) a derivative, analog or antigen-binding fragment thereof.

In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is an antibody or fragment thereof that immunospecifically binds to integrin α_{V}β₃ and wherein at least one of the TNF-α antagonists is a soluble TNF-α receptor such as etanercept (ENBREL™; Immunex) or a fragment, derivative or analog thereof, or an antibody that immunospecifically binds to TNF-α such as infliximab (REMICADE™; Centacor) a derivative, analog or antigen-binding fragment thereof.

In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more TNF-α antagonists, wherein at least one of the integrin α_{V}β₃ antagonists is VITAXIN™ or an antigen-binding fragment thereof and wherein at least one of the TNF-α antagonists is a soluble TNF-α receptor such as etanercept (ENBREL™; Immunex) or a fragment, derivative or analog thereof, or an antibody that immunospecifically binds to TNF-α such as infliximab (REMICADE™; Centacor) a derivative, analog or antigen-binding fragment thereof.

The present invention provides methods of preventing, treating, managing or ameliorating one or more symptoms associated with an inflammatory disorder or an autoimmune disorder associated with inflammation in a subject, said methods comprising administering to said subject one or more integrin α_{V}β₃ antagonists and one or more anti-inflammatory agents. Section 5.2 provides non-limiting examples of anti-inflammatory agents which can be used in accordance with the methods of the invention.

In a specific embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more anti-inflammatory agents. In another embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more anti-inflammatory agents, wherein at least one of the integrin α_{V}β₃ antagonists is an antibody or fragment thereof that immunospecifically binds to integrin α_{V}β₃.

In a preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of one or more integrin α_{V}β₃ antagonists and a prophylactically or therapeutically effective amount of one or more anti-inflammatory agents, wherein at least one of the integrin α_{V}β₃ antagonists is VITAXIN™ or an antigen-binding fragment thereof. In another preferred embodiment, the present invention provides a method for preventing, treating, managing or ameliorating one or more symptoms associated with an autoimmune or inflammatory disorder in a subject, said method comprising administering to said subject a prophylactically or therapeutically effective amount of VITAXIN™ or an antigen-binding fragment thereof and a prophylactically or therapeutically effective amount of one or more anti-inflammatory agents.

The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, and one or more immunomodulatory agents. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept), and a prophylactically or therapeutically effective amount of methotrexate. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of methotrexate. The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, and one or more CD2 binding molecules. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically Ieffective amount of a soluble TNF-α receptor (e.g., entanercept), and a prophylactically or therapeutically effective amount of MEDI-507 or antigen-binding fragment thereof. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of MEDI-507 or antigen-binding fragment thereof.

The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, and one or more anti-inflammatory agents. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept), and a prophylactically or therapeutically effective amount of a steriodal or non-steroidal anti-inflammatory drug. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of an antibody that immunospecifically binds to TNF-α (e.g., infliximab or an antigen-binding fragment thereof), and a prophylactically or therapeutically effective amount of a steriodal or non-steroidal anti-inflammatory drug.

The present invention provides methods of preventing, treating, managing or ameliorating one or more symptoms associated with an autoimmune or inflammatory disorder in a subject, said method comprising administering to said subject one or more integrin α_{V}β₃ antagonists, one or more TNF-α antagonists, one or more immunomodulatory agents, and one or more anti-inflammatory agents. In a specific embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept) or an antibody that immunospecifically binds to TNF-α (e.g., infliximab or an antigen-binding fragment thereof), a prophylactically or therapeutically effective amount of methotrexate, and a prophylactically or therapeutically effective amount of a steriodal or non-steroidal anti-inflammatory drug. In another embodiment, the present invention provides a method for preventing, treating, managing or ameliorating one or more symptoms associated with an autoimmune or inflammatory disorder, said method comprising administering to said subject a prophylactically or therapeutically effective amount of VITAXIN™, a prophylactically or therapeutically effective amount of a soluble TNF-α receptor (e.g., entanercept) or an antibody that immunospecifically binds to TNF-α (e.g., infliximab or an antigen-binding fragment thereof), a prophylactically or therapeutically effective amount of a CD2 binding molecule (e.g., MEDI-507 or an antigen-binding fragment thereof), and a prophylactically or therapeutically effective amount of a steriodal or non-steroidal anti-inflammatory drug.

The present invention provides methods of preventing, treating, managing or ameliorating one or more symptoms associated with an autoimmune or inflammatory disorder in a subject, said methods comprising administering to said subject one or more integrin α_{V}β₃ antagonists and one or more nucleic acid molecules encoding one or more prophylactic or therapeutic agents other than integrin α_{V}β₃ antagonists. The present invention also provides methods of preventing, treating, managing or ameliorating one or more symptoms associated with an autoimmune or inflammatory disorder in a subject, said methods comprising administering to said subject one or more nucleic acid molecules encoding one or more integrin α_{V}β₃ antagonists and one or more nucleic acid molecules encoding one or more prophylactic or therapeutic agents other than integrin α_{V}β₃ antagonists. The present invention further provides methods of preventing, treating, managing or ameliorating one or more symptoms associated with an autoimmune or inflammatory disorder in a subject, said methods comprising administering to said subject one or more nucleic acid molecules encoding one or more integrin α_{V}β₃ antagonists and one or more nucleic acid molecules encoding one or more prophylactic or therapeutic agents other than integrin α_{V}β₃ antagonists. The methods of the invention are particularly useful for the prevention or treatment of rheumatoid arthritis, spondyloarthropathies (e.g., psoriatic arthritis, ankylosing spondylitis, Reiter's Syndrome (a.k.a., reactive arthritis), inflammatory bowel disease associated arthritis, and undifferentitated spondyloarthropathy), psoriasis, undifferentiated arthropathy, and arthritis. The methods of the invention can also be applied to the prevention, treatment, management or amelioration of one or more symptoms associated with inflammatory osteolysis, other disorders characterized by abnormal bone reabsorption, or disorder characterized by bone loss (e.g., osteoporosis).

In a preferred embodiment, the invention provides methods for the prevention, treatment, management or amelioration of one or more symptoms associated with rheumatoid arthritis, arthritis, psoriatic arthritis or psoriasis. In another preferred embodiment, the invention provides methods for the prevention, treatment, management or amelioration of one or more symptoms associated with psoriasis or psoriatic arthritis. In yet another preferred embodiment, the invention provides methods for the prevention, treatment, management, or amelioration of the symptoms of osteoporosis which are associated with rheumatoid arthritis, psoriatic arthritis or psoriasis, and juvenile chronic arthritis.

The invention encompasses methods for treating or ameliorating one or more symptoms of an autoimmune or inflammatory disorder in a subject refractory to conventional therapies for such a disorder, said methods comprising administering to said subject one or more integrin α_{V}β₃ antagonists or a pharmaceutical composition comprising one or more integrin α_{V}β₃ antagonists. The invention also encompasses methods for treating or ameliorating one or more symptoms of an autoimmune or inflammatory disorder in a subject refractory to existing single agent therapies for such a disorder, said methods comprising administering to said subject one or more integrin α_{V}β₃ antagonists and one or more prophylactic or therapeutic agents other than integrin α_{V}β₃ antagonists. Further, the invention encompasses methods for treating or ameliorating one or more symptoms of an autoimmune or inflammatory disorder in a subject refractory to existing single agent therapies for such a disorder, said methods comprising administering to said subject a pharmaceutical composition comprising one or more integrin α_{V}β₃ antagonists and one or more prophylactic or therapeutic agents other than integrin α_{V}β₃ antagonists.

In a specific embodiment, the invention provides methods for treating an autoimmune or inflammatory disorder comprising administering an integrin α_{V}β₃ antagonist and a prophylactic or therapeutic agent other than an integrin α_{V}β₃ antagonist to subjects who have proven refractory to other treatments but are no longer on these treatments. In a preferred embodiment, the invention provides methods for treating rheumatoid arthritis, arthritis, psoriasis or psoriatic arthritis comprising administering an integrin α_{V}β₃ antagonist and a prophylactic or therapeutic agent other than an integrin α_{V}β₃ antagonist to subjects who have proven refractory to other treatments but are no longer on these treatments.

The invention provides methods for treating an autoimmune or inflammatory disorder comprising administering an integrin α_{V}β₃ antagonist to subjects being treated with methotrexate and an TNF-α antagonist. Among these subjects are those with persistent active disease (i. e., refractory patients) and those with mild disease activity despite treatment with methotrexate and an TNF-α antagonist. The invention also provides methods for preventing the recurrence of one or more symptoms of an autoimmune or inflammatory disorder comprising administering an integrin α_{V}β₃ antagonist to subjects who have been treated with methotrexate and an TNF-α antagonist (e.g., REMICADE™ or ENBREL™) and have no disease activity.

The invention provides methods for treating an autoimmune or inflammatory disorder comprising administering an integrin α_{V}β₃ antagonist to subjects taking methotrexate that have not received an TNF-α antagonist. Among these subjects are subjects with no disease activity, subjects with persistent active disease, and subjects with mild disease activity. Among these subjects are also subjects concurrently treated with other prophylactic and/or therapeutic agents but not an TNF-α antagonist. Also among these subjects are subjects only being treated with methotrexate.

The invention provides methods for treating an autoimmune or inflammatory disorder comprising administering an integrin α_{V}β₃ antagonist to subjects being treated with a prophylactic or therapeutic agent other than methotrexate. Among these subjects are subjects treated with a TNF-α antagonist (e.g., REMICADE™ or ENBREL™) and subjects not being treated with a TNF-α antagonist but some other prophylactic or therapeutic agent.

The invention encompasses methods for preventing the occurrence of an autoimmune or inflammatory disorder, or one or more symptoms thereof in a subject predisposed to said disorder, said methods comprising administering to said subject one or more integrin α_{V}β₃ antagonists and one or more prophylactic or therapeutic agents other than integrin α_{V}β₃ antagonists. In a specific embodiment, the invention provides methods for preventing the occurrence of rheumatoid arthritis, psoriatic arthritis or psoriasis, or one or more symptoms thereof in a subject predisposed to such a disorder, said methods comprising administering to said subject one or more integrin α_{V}β₃ antagonists and one or more prophylactic or therapeutic agents other than integrin α_{V}β₃ antagonists.

The invention encompasses methods for preventing the occurrence of an autoimmune or inflammatory disorder, or one or more symptoms thereof in a subject predisposed to said disorder, said methods comprising administering to said subject a pharmaceutical composition one or more integrin α_{V}β₃ antagonists and one or more prophylactic or therapeutic agents other than integrin α_{V}β₃ antagonists. In a specific embodiment, the invention provides methods for preventing the occurrence of rheumatoid arthritis, psoriatic arthritis or psoriasis, or one or more symptoms thereof in a subject predisposed to such a disorder, said methods comprising administering to said subject a pharmaceutical composition one or more integrin α_{V}β₃ antagonists and one or more prophylactic or therapeutic agents other than integrin α_{V}β₃ antagonists.

5.4. Compositions and Methods of Administering Combination Therapy

The present invention provides compositions for the treatment, prophylaxis, and amelioration of one or more symptoms associated with an autoimmune or inflammatory disorder. In a specific embodiment, a composition comprises one or more integrin α_{V}β₃ antagonists. In another embodiment, a composition comprises one or more nucleic acid molecules encoding one or more integrin α_{V}β₃ antagonists. In another embodiment, a composition comprises one or more integrin α_{V}β₃ antagonists and one or more prophylactic or therapeutic agents other than integrin α_{V}β₃ antagonists, said prophylactic or therapeutic agents known to be useful for, or having been or currently being used in the prevention, treatment or amelioration of one or more symptoms associated an autoimmune or inflammatory disorder. In another embodiment, a composition comprises one or more nucleic acid molecules encoding one or more integrin α_{V}β₃ antagonists and one or more prophylactic or therapeutic agents other than integrin α_{V}β₃ antagonists, said prophylactic or therapeutic agents known to be useful for, or having been or currently being used in the prevention, treatment or amelioration of one or more symptoms associated an autoimmune or inflammatory disorder. In another embodiment, a composition comprises one or more integrin α_{V}β₃ antagonists and one or more nucleic acid molecules encoding one or more prophylactic or therapeutic agents other than integrin α_{V}β₃ antagonists, said prophylactic or therapeutic agents known to be useful for, or having been or currently being used in the prevention, treatment or amelioration of one or more symptoms associated an autoimmune or inflammatory disorder. In yet another embodiment, a composition comprises one or more nucleic acid molecules encoding one or more integrin α_{V}β₃ antagonists and one or more nucleic acid molecules encoding one or more prophylactic or therapeutic agents other than integrin α_{V}β₃ antagonists, said prophylactic or therapeutic agents known to be useful for, or having been or currently being used in the prevention, treatment or amelioration of one or more symptoms associated an autoimmune or inflammatory disorder.

In a specific embodiment, a composition comprises a one or more integrin α_{V}β₃ antagonists and one or more immunomodulatory agents. In another embodiment, a composition comprises VITAXIN™ and one or more immunomodulatory agents. In another embodiment, a composition comprises VITAXIN™ and methotrexate. In another embodiment, a composition comprises a one or more integrin α_{V}β₃ antagonists and one or more CD2 antagonists. In another embodiment, a composition comprises VITAXIN™ and one or more CD2 antagonists. In another embodiment, a composition comprises one or more integrin α_{V}β₃ antagonists and one or more CD2 binding molecules. In yet another embodiment, a composition comprises VITAXINT or an antigen-binding fragment thereof and one or more CD2 binding molecules. In a preferred embodiment, a composition comprises VITAXIN™ or an antigen-binding fragment thereof and MEDI-507 or an antigen-binding fragment thereof.

In a specific embodiment, a composition comprises one or more integrin α_{V}β₃ antagonists and one or more anti-angiogenic agents. In another embodiment, a composition comprises VITAXIN™ or an antigen-binding fragment thereof and one or more anti-angiogenic agents.

In a specific embodiment, a composition comprises one or more integrin α_{V}β₃ antagonists and one or more TNF-α antagonists. In another embodiment, a composition comprises VITAXIN™ or an antigen-binding fragment thereof and one or more TNF-α antagonists. In a preferred embodiment, a composition comprises VITAXIN™ or an antigen-binding fragment thereof and a soluble TNF-α receptor (e.g., etanercept) or an antibody that immunospecifically binds to TNF-α.

In a specific embodiment, a composition comprises one or more integrin α_{V}β₃ antagonists and one or more anti-inflammatory agents. In another embodiment, a composition comprises VITAXIN™ or an antigen-binding fragment thereof and one or more anti-inflammatory agents. In a preferred embodiment, a composition comprises VITAXIN™ or an antigen-binding fragment thereof and a steriodal or non-steriodal anti-inflammatory drug.

In one embodiment, a composition comprises one or more integrin α_{V}β₃ antagonists, one or more immunomodulatory agents, and one or more TNF-α antagonists. In another embodiment, a composition comprises one or more integrin α_{V}β₃ antagonists, one or more CD2 binding molecules, and one or more TNF-α antagonists. In another embodiment, a composition comprises one or more integrin α_{V}β₃ antagonists, one or more anti-inflammatory agents, and one or more TNF-α antagonists. In accordance with these embodiments, preferably, at least one of the integrin α_{V}β₃ antagonists is VITAXIN™ or an antigen-binding fragment thereof.

In a preferred embodiment, a composition of the invention is a pharmaceutical composition. Such compositions comprise a prophylactically or therapeutically effective amount of one or more prophylactic or therapeutic agents (e.g., an integrin α_{V}β₃ antagonist or other prophylactic or therapeutic agent), and a pharmaceutically acceptable carrier. In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “carrier” refers to a diluent, adjuvant (e.g., Freund's adjuvant (complete and incomplete)), excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is a preferred carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard carriers such as pharmaceutical grades of mannitol, lactose, starch, magnesium stearate, sodium saccharine, cellulose, magnesium carbonate, etc. Examples of suitable pharmaceutical carriers are described in “Remington's Pharmaceutical Sciences” by E. W. Martin. Such compositions will contain a prophylactically or therapeutically effective amount of a prophylactic or therapeutic agent preferably in purified form, together with a suitable amount of carrier so as to provide the form for proper administration to the patient. The formulation should suit the mode of administration. In a preferred embodiment, the pharmaceutical compositions are sterile and in suitable form for administration to a subject, preferably an animal subject, more preferably a mammalian subject, and most preferably a human subject.

In a specific embodiment, it may be desirable to administer the pharmaceutical compositions of the invention locally to the area in need of treatment; this may be achieved by, for example, and not by way of limitation, local infusion, by injection, or by means of an implant, said implant being of a porous, non-porous, or gelatinous material, including membranes, such as sialastic membranes, or fibers. Preferably, when administering one or more prophylactic or therapeutic agents, care must be taken to use materials to which the prophylactic or therapeutic agents do not absorb.

In another embodiment, the composition can be delivered in a vesicle, in particular a liposome (see Langer, Science 249:1527-1533 (1990); Treat et al., in Liposomes in the Therapy of Infectious Disease and Cancer, Lopez-Berestein and Fidler (eds.), Liss, N.Y., pp. 353-365 (1989); Lopez-Berestein, ibid., pp. 3 17-327; see generally ibid.).

In yet another embodiment, the composition can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of the antibodies of the invention or fragments thereof (see e.g., Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, N.Y. (1984); Ranger and Peppas, 1983, J., Macromol. Sci. Rev. Macromol. Chem. 23:61; see also Levy et al., 1985, Science 228:190; During et al., 1989, Ann. Neurol. 25:351; Howard et al., 1989, J. Neurosurg. 7 1:105); U.S. Pat. Nos. 5,679,377; 5,916,597; 5,912,015; 5,989,463; 5,128,326; PCT Publication No. WO 99/15154; and PCT Publication No. WO 99/20253. Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In a preferred embodiment, the polymer used in a sustained release formulation is inert, free of leachable impurities, stable on storage, sterile, and biodegradable. In yet another embodiment, a controlled or sustained release system can be placed in proximity of the therapeutic target, i. e., the lungs, thus requiring only a fraction of the systemic dose (see, e.g., Goodson, in Medical Applications of Controlled Release, supra, vol. 2, pp. 115-138 (1984)).

Controlled release systems are discussed in the review by Langer (1990, Science 249:1527-1533). Any technique known to one of skill in the art can be used to produce sustained release formulations comprising one or more antibodies of the invention or fragments thereof. See, e.g.,. U.S. Pat. No. 4,526,938, .PCT publication WO 91/05548, PCT publication WO 96/20698,.Ning et al., 1996, “Intratumoral Radioimmunotheraphy of a Human Colon Cancer Xenograft Using a Sustained-Release Gel,” Radiotherapy & Oncology 39:179-189,.Song et al., 1995, “Antibody Mediated Lung Targeting of Long-Circulating Emulsions,” PDA Journal of Pharmaceutical Science & Technology 50:372-397, Cleek et al., 1997, “Biodegradable Polymeric Carriers for a bFGF Antibody for Cardiovascular Application,” Pro. Int'l. Symp. Control. Rel. Bioact. Mater. 24:853-854, and Lam et al., 1997, “Microencapsulation of Recombinant Humanized Monoclonal Antibody for Local Delivery,” Proc. Int'l. Symp. Control Rel. Bioact. Mater. 24:759-760, each of which is incorporated herein by reference in their entirety.

In a specific embodiment where the composition of the invention is one or more nucleic acid molecules encoding one or more prophylactic or therapeutic agents, the nucleic acid can be administered in vivo to promote expression of its encoded prophylactic or therapeutic agents, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell-surface receptors or transfecting agents, or by administering it in linkage to a homeobox-like peptide which is known to enter the nucleus (see e.g., Joliot et al., 1991, Proc. Natl. Acad. Sci. USA 88:1864-1868), etc. Alternatively, a nucleic acid can be introduced intracellularly and incorporated within host cell DNA for expression by homologous recombination.

A pharmaceutical composition of the invention is formulated to be compatible with its intended route of administration. Examples of routes of administration include, but are not limited to, parenteral, e.g., intravenous, intradermal, subcutaneous, oral (e.g., inhalation), intranasal, transdermal (topical), transmucosal, and rectal administration. In a specific embodiment, the composition is formulated in accordance with routine procedures as a pharmaceutical composition adapted for intravenous, subcutaneous, intramuscular, oral, intranasal or topical administration to human beings. In a preferred embodiment, a pharmaceutical composition is formulated in accordance with routine procedures for subcutaneous administration to human beings. Typically, compositions for intravenous administration are solutions in sterile isotonic aqueous buffer. Where necessary, the composition may also include a solubilizing agent and a local anesthetic such as lignocamne to ease pain at the site of the injection.

If the compositions of the invention are to be administered topically, the compositions can be formulated in the form of, e.g., an ointment, cream, transdermal patch, lotion, gel, shampoo, spray, aerosol, solution, emulsion, or other form well-known to one of skill in the art. See, e.g., Remington's Pharmaceutical Sciences and Introduction to Pharmaceutical Dosage Forms, 4^{th} ed., Lea & Febiger, Philadelphia, Pa. (1985). For non-sprayable topical dosage forms, viscous to semi-solid or solid forms comprising a carrier or one or more excipients compatible with topical application and having a dynamic viscosity preferably greater than water are typically employed. Suitable formulations include, without limitation, solutions, suspensions, emulsions, creams, ointments, powders, liniments, salves, and the like, which are, if desired, sterilized or mixed with auxiliary agents (e.g., preservatives, stabilizers, wetting agents, buffers, or salts) for influencing various properties, such as, for example, osmotic pressure. Other suitable topical dosage forms include sprayable aerosol preparations wherein the active ingredient, preferably in combination with a solid or liquid inert carrier, is packaged in a mixture with a pressurized volatile (e.g., a gaseous propellant, such as freon), or in a squeeze bottle. Moisturizers or humectants can also be added to pharmaceutical compositions and dosage forms if desired. Examples of such additional ingredients are well-known in the art.

If the compositions of the invention are to be administered intranasally, the compositions can be formulated in an aerosol form, spray, mist or in the form of drops. In particular, prophylactic or therapeutic agents for use according to the present invention can be conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol the dosage unit may be determined by providing a valve to deliver a metered amount. Capsules and cartridges of, e.g., gelatin for use in an inhaler or insufflator may be formulated containing a powder mix of the compound and a suitable powder base such as lactose or starch.

If the compositions of the invention are to be administered orally, the compositions can be formulated orally in the form of, e.g., tablets, capsules, cachets, gelcaps, solutions, suspensions and the like. Tablets or capsules can be prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulphate). The tablets may be coated by methods well-known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). The preparations may also contain buffer salts, flavoring, coloring and sweetening agents as appropriate. Preparations for oral administration may be suitably formulated for slow release, controlled release or sustained release of a prophylactic or therapeutic agent(s).

The compositions of the invention may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Alternatively, the active ingredient may be in powder form for constitution with a suitable vehicle, e.g., sterile pyrogen-free water, before use.

The compositions of the invention may also be formulated in rectal compositions such as suppositories or retention enemas, e.g., containing conventional suppository bases such as cocoa butter or other glycerides.

In addition to the formulations described previously, the compositions of the invention may also be formulated as a depot preparation. Such long acting formulations may be administered by implantation (for example subcutaneously or intramuscularly) or by intramuscular injection. Thus, for example, the compositions may be formulated with suitable polymeric or hydrophobic materials (for example as an emulsion in an acceptable oil) or ion exchange resins, or as sparingly soluble derivatives, for example, as a sparingly soluble salt.

The compositions of the invention can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

Generally, the ingredients of compositions of the invention are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

In particular, the invention provides that one or more of the prophylactic or therapeutic agents, or pharmaceutical compositions of the invention is packaged in a hermetically sealed container such as an ampoule or sachette indicating the quantity of the agent. In one embodiment, one or more of the prophylactic or therapeutic agents, or pharmaceutical compositions of the invention is supplied as a dry sterilized lyophilized powder or water free concentrate in a hermetically sealed container and can be reconstituted, e.g., with water or saline to the appropriate concentration for administration to a subject. Preferably, one or more of the prophylactic or therapeutic agents, or pharmaceutical compositions of the invention is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. The lyophilized prophylactic or therapeutic agents, or pharmaceutical compositions of the invention should be stored at between 2 and 8° C. in its original container and the prophylactic or therapeutic agents, or pharmaceutical compositions of the invention should be administered within 1 week, preferably within 5 days, within 72 hours, within 48 hours, within 24 hours, within 12 hours, within 6 hours, within 5 hours, within 3 hours, or within 1 hour after being reconstituted. In an alternative embodiment, one or more of the prophylactic or therapeutic agents, or pharmaceutical compositions of the invention is supplied in liquid form in a hermetically sealed container indicating the quantity and concentration of the agent. Preferably, the liquid form of the administered composition is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml. The liquid form should be stored at between 2° C. and 8° C. in its original container.

In a preferred embodiment of the invention, REMICADE™ is supplied as a sterile and lyophilized powder for intravenous infusion to be reconstituted with 10 ml sterile water for injection. Each single-use vial of REMICADE™ contains 100 mg infliximab, 500 mg sucrose, 0.5 mg polysorbate 80, 2.2 mg monobasic sodium phosphate and 6.1 mg dibasic sodium phosphate. According to The Physician's Desk Reference (55^{th} ed., 2001), the total dose of the reconstituted product must be further diluted to 250 ml with 0.9% Sodium Chloride Injection, USP, with the infusion concentration ranging between 0.4 mg/ml and 4 mg/ml.

In another preferred embodiment of the invention, ENBREL™ is supplied as a sterile, preservative-free, lyophilized powder for parenteral administration after reconstitution with 1 ml of supplied Sterile Bacteriostatic Water for Injection, USP (containing 0.9% benzyl alcohol). According to The Physician's Desk Reference (55^{th} ed., 2001) Each single-use vial of ENBREL™ contains 25 mg etanercept, 40 mg mannitol, 10 mg sucrose, and 1.2 mg tromethamine.

In yet other preferred embodiments of the invention, VITAXIN™ is formulated at 1 mg/ml, 5 mg/ml, 10 mg/ml, and 25 mg/ml for intravenous injections and at 5 mg/ml, 10 mg/ml, 80 mg/ml or 100 mg/ml for repeated subcutaneous administration.

In other preferred embodiments of the invention, methotrexate is formulated at 25 mg/ml and supplied in vials, for example, at 1 mL, 2 mL and 10 mL. Methotrexate for injection contains methotrexate sodium equivalent to 50 mg and 250 mg methotrexate respectively, with 90% w/v Benzyl Alcohol as a preservative and 0.260% w/v Sodium Chloride and water for injection. Methotrexate can be given by injection by intramuscular, intravenous, intraarterial using the preservative formulation which contains Benzyl Alcohol. Methotrexate can be given by intrathecal route using the non-preservative formulation. In other embodiments of the invention, methotrexate is supplied as a tablet with a unit dose of 2.5 mg methotrexate sodium.

In yet other preferred embodiments, the invention provides that MEDI-507 is packaged in a hermetically sealed container such as an ampoule or sachette indicating the quantity of MEDI-507. In one embodiment, MEDI-507 is supplied as a dry sterilized lyophilized powder or water free concentrate in a hermetically sealed container and can be reconstituted, e.g., with water or saline to the appropriate concentration for administration to a subject. Preferably, MEDI-507 is supplied as a dry sterile lyophilized powder in a hermetically sealed container at a unit dosage of at least 5 mg, more preferably at least 10 mg, at least 15 mg, at least 25 mg, at least 35 mg, at least 45 mg, at least 50 mg, at least 75 mg, or at least 100 mg. In an alternative embodiment, MEDI-507 is supplied in liquid form in a hermetically sealed container indicating the quantity and concentration of the MEDI-507. Preferably, the liquid form of MEDI-507 is supplied in a hermetically sealed container at least 0.25 mg/ml, more preferably at least 0.5 mg/ml, at least 1 mg/ml, at least 2.5 mg/ml, at least 5 mg/ml, at least 8 mg/ml, at least 10 mg/ml, at least 15 mg/kg, at least 25 mg/ml, at least 50 mg/ml, at least 75 mg/ml or at least 100 mg/ml.

The compositions may, if desired, be presented in a pack or dispenser device that may contain one or more unit dosage forms containing the active ingredient. The pack may for example comprise metal or plastic foil, such as a blister pack. The pack or dispenser device may be accompanied by instructions for administration. In certain preferred embodiments, the pack or dispenser contains one or more unit dosage forms containing no more than 25 mg ENBREL, 2.5 mg METHOTREXATE, 100 mg REMICADE™ and 5 mg/mL VITAXIN™.

Generally, the ingredients of the compositions of the invention are derived from a subject that is the same species origin or species reactivity as recipient of such compositions. Thus, in a preferred embodiment, human or humanized antibodies are administered to a human patient for therapy or prophylaxis.

The amount of the composition of the invention which will be effective in the treatment, prevention or amelioration of one or more symptoms associated with an inflammatory disease or autoimmune disorder can be determined by standard clinical techniques. The precise dose to be employed in the formulation will also depend on the route of administration, and the seriousness of the condition, and should be decided according to the judgment of the practitioner and each patient's circumstances. Effective doses may be extrapolated from dose-response curves derived from in vitro or animal model test systems.

For antibodies, proteins, polypeptides, peptides and fusion proteins encompassed by the invention, the dosage administered to a patient is typically 0.0001 mg/kg to 100 mg/kg of the patient's body weight. Preferably, the dosage administered to a patient is between 0.0001 mg/kg and 20 mg/kg, 0.0001 mg/kg and 10 mg/kg, 0.0001 mg/kg and 5 mg/kg, 0.0001 and 2 mg/kg, 0.0001 and 1 mg/kg, 0.0001 mg/kg and 0.75 mg/kg, 0.0001 mg/kg and 0.5 mg/kg, 0.0001 mg/kg to 0.25 mg/kg, 0.0001 to 0.15 mg/kg, 0.0001 to 0.10 mg/kg, 0.001 to 0.5 mg/kg, 0.01 to 0.25 mg/kg or 0.01 to 0.10 mg/kg of the patient's body weight. Generally, human antibodies have a longer half-life within the human body than antibodies from other species due to the immune response to the foreign polypeptides. Thus, lower dosages of human antibodies and less frequent administration is often possible. Further, the dosage and frequency of administration of antibodies of the invention or fragments thereof may be reduced by enhancing uptake and tissue penetration of the antibodies by modifications such as, for example, lipidation.

In a specific embodiment, the dosage of the composition of the invention or a prophylactic or therapeutic agent administered to prevent, treat or ameliorate one or more symptoms associated with an autoimmune or inflammatory disorder in a patient is 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.5 μg/kg or less of a patient's body weight. In another embodiment, the dosage of the composition of the invention or a prophylactic or therapeutic agent administered to prevent, treat or ameliorate one or more symptoms associated with an autoimmune or inflammatory disorder in a patient is a unit dose of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In one embodiment, the recommended dosage of ENBREL™ is 0.01 to 10 mg/kg, preferably 0.1 to 10 mg/kg, more preferably 0.1 to 5 mg/kg, and even more preferably 0.5 to 2 mg/kg. In another embodiment of the invention, the recommended dose of ENBREL™ is 0.01 to 10 mg/kg/week, more preferably 0.1 to 5 mg/kg/week, even more preferably 0.5 to 2 mg/kg/week. In a most preferred embodiment, the weekly dose is not to exceed 50 mg/week. In preferred embodiments, ENBREL™ is administrated by subcutaneous injection twice a week.

In a preferred embodiment of the invention, ENBREL™ is administered at a dose of about 1 mg to about 50 mg, more preferably about 10 mg to about 40 mg, most preferably about 20 mg to about 30 mg. In certain embodiments, an antagonist of Integrin α_{V}β₃ is administered in combination with the administration of 0.1 mg to 1 mg, 1 mg to 5 mg, 5 mg to 10 mg, 10 mg to 15 mg, 15 mg to 20 mg, 20 mg to 25 mg, 25 mg to 30 mg, 30 mg to 35 mg,35 mg to 40 mg, 40 mg to 45 mg, 45 mg to 50 mg, 50 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, 95 mg to 100 mg, 100 mg to 105 mg, 105 mg to 110 mg, 110 mg to 115 mg, or 115 mg to 120 mg of ENBREL™ per week. Preferably, ENBREL™ is given twice weekly as a subcutaneous injection. Preferably the injections are administered 72 to 96 hours apart. In an embodiment, the injections are administered 36 to 132 hours apart, preferably 48 to 114 hours apart, more preferably 72 to 96 hours apart, even more preferably about 84 hours apart. In a preferred embodiment, the dosage amounts of ENBREL™ are less than are typical when it is administered alone. See Physicians' Desk Reference (55^{th} ed. 2001). Accordingly, in a preferred embodiment, the administration of an antagonist of Integrin α_{V}β₃ is combined with the administration of no more than 25 mg of ENBREL™. In preferred embodiments, less than 25 mg, less than 20 mg, less than 15 mg, less than 10 mg or less than 5 mg ENBREL™ is administered per dose. According to the methods of the invention, ENBREL™ is administered at doses of 1 mg, 1 mg to 5 mg, 5 mg to 10 mg, 10 mg to 15 mg, 15 mg to 20 mg, 20 mg to 25 mg, or 25 mg, twice weekly. Preferably, the Integrin α_{V}β₃ antagonist is VITAXIN™.

In other embodiments of the invention, an integrin α_{V}β₃ antagonist is administered in combination with anti-TNF-α antibodies. Preferably, the anti-TNF-α antibody is infliximab (REMICADE™). In an embodiment of the invention, a recommended dose of REMICADE™ is 0.1 to 10 mg/kg, more preferably 1 to 7 mg/kg, even more preferably 2 to 6 mg/kg, and most preferably 3 to 5 mg/kg. In a most preferred embodiment, the dose does not exceed 3 mg/kg. In certain preferred embodiments, REMICADE™ is administrated by intravenous infusion followed with an additional dose at 2 and 6 weeks after the first infusion then every 8 weeks thereafter.

In a preferred embodiment of the invention, REMICADE™ is administered at a dose of about lmg to about 600 mg, more preferably about 100 mg to 500 mg, and most preferably about 200 mg to about 400 mg. In certain embodiments of the invention, an integrin α_{V}β₃ antagonist is administered in combination with 1 mg to 10 mg, 10 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, 400 mg to 450 mg, 450 mg to 500 mg, 550 mg to 600 mg, 600 mg to 650 mg, 650 mg to 700 mg, 700 mg to 750 mg, 750 mg to 800 mg, 800 mg to 850 mg, 850 mg to 900 mg, 900 mg to 950 mg, 950 mg to 1000 mg of REMICADE™, initially and at 2 and 6 weeks after the first dose, and then every 8 weeks after. In preferred embodiments, the dosage amounts for REMICADE™ are less than are typical when it is administered alone. See Physicians' Desk Reference (55^{th} ed. 2001). Accordingly, in a preferred embodiment, no more than 600 mg of REMICADE™ is given as an intravenous infusion followed with additional doses at 2 and 6 weeks after the first infusion then every 8 weeks thereafter. In other embodiments, the additional doses are administered at 1 to 12 weeks, preferably 4 to 12 weeks, more preferably 6 to 12 weeks, and even more preferably 8 to 12 weeks. Preferably, the integrin α_{V}β₃ antagonist is VITAXIN™.

In certain embodiments of the invention, an integrin α_{V}β₃ antagonist is administered in combination with the administration of methotrexate alone or in combination with other prophylactic and/or therapeutic agents. In certain embodiments, the recommended dose of methotrexate is 0.01 to 3 mg/kg, more preferably 0.1 to 2 mg/kg and most preferably 0.5 to 1 mg/kg. In certain preferred embodiments, the recommended dose of methotrexate is 0.01 to 3 mg/kg/week, more preferably 0.1 to 2 mg/kg/week and most preferably 0.5 to 1 mg/kg/week. In a most preferred embodiment, the weekly dose does not exceed 20 g/week.

In a preferred embodiment, methotrexate is administered at a dose of about 0.01 mg to about 70 mg, preferably about 1 mg to 60 mg, most preferably about 10 mg to 60 mg. Methotrexate is administered at 0.5 mg to 1 mg, 1 mg to 1.5 mg, 1.5 mg to 2 mg, 2 mg to 2.5 mg, 2.5 mg to 3 mg, 3 mg to 3.5 mg, 3.5 mg to 4 mg, 4 mg to 4.5 mg, 4.5 mg to 5 mg, 5 mg to 5.5 mg, 5.5 gm to 6 mg, 6 mg to 6.5 mg, 6.5 mg to 7 mg, 7 mg to 7.5 mg, 7.5 mg to 8 mg, 8 mg to 8.5 mg, 8.5 mg to 9 mg, 9 mg to 9.5 mg, 9.5 mg to 10 mg, 10 mg to 10.5 mg, 10.5 mg to 11 mg, 11 mg to 12 mg, 12 mg to 13 mg, 13 mg to 14 mg, 14 mg to 15 mg, 15 mg to 20 mg, 20mg to 25 mg, 25 mg to 30 mg, 30 mg to 35 mg, 35 mg to 40 mg, 40 mg to 45 mg, 45 mg to 50 mg, 50 mg to 60 mg, 60 mg to 70 mg, 70 mg to 80 mg. In a preferred embodiment, the dosage amounts of methotrexate administered are less than are typical when it is administered alone. See Physicians' Desk Reference (55^{th} ed. 2001). Accordingly, in a preferred embodiment of the invention, an Integrin α_{V}β₃ antagonist is administered in combination with the concurrent oral or intramuscular administration of no more than 57 mg methotrexate once weekly or no more than 2.5 mg every 12 hours for 3 doses/week. In a more preferable embodiment of the invention, an Integrin α_{V}β₃ antagonist is administered in combination with the concurrent oral or intramuscular administration of no more than 20 mg methotrexate per week. In certain embodiments of the invention, methotrexate is administered 6 to 12 hours apart, 12 to 18 hours apart, 18 to 24 hours part, 24 to 36 hours apart, 36 to 48 hours apart, 48 to 52 hours apart, 52 to 60 hours apart, 60 to 72 hours apart, 72 to 84 hours apart, 84 to 96 hours apart, or 96 to 120 hours apart. In a most preferred embodiment of the invention, an Integrin α_{V}β₃ antagonist is administered in combination with the concurrent oral administration of no more than 15-20 mg methotrexate as one dose per week In other embodiments, methotrexate is administered no more than once per week, once per every two weeks, once per every 3 weeks or once per month.

In certain embodiments, the dose of VITAXIN™ administered to a subject is 0.1 to 10 mg/kg, preferably 1 to 9 mg/kg, more preferably 2 to 8 mg, even more preferably 3 to 7 mg/kg, and most preferably 4 to 6 mg/kg. In other preferred embodiments, the dose of VITAXIN™ administered to a subject is 0.1 to 10 mg/kg/week, preferably 1 to 9 mg/kg/week, more preferably 2 to 8 mg/week, even more preferably 3 to 7 mg/kg/week, and most preferably 4 to 6 mg/kg/week.

In other embodiments, a subject is administered one or more doses of 200 μg/kg or less, 150 μg/kg or less, preferably 125 μg/kg or less, 100 μg/kg or less, 95 μg/kg or less, 90 μg/kg or less, 85 μg/kg or less, 80 μg/kg or less, 75 μg/kg or less, 70 μg/kg or less, 65 μg/kg or less, 60 μg/kg or less, 55 μg/kg or less, 50 μg/kg or less, 45 μg/kg or less, 40 μg/kg or less, 35 μg/kg or less, 30 μg/kg or less, 25 μg/kg or less, 20 μg/kg or less, 15 μg/kg or less, 10 μg/kg or less, 5 μg/kg or less, 2.5 μg/kg or less, 2 μg/kg or less, 1.5 μg/kg or less, 1 μg/kg or less, 0.5 μg/kg or less, or 0.4 μg/kg or less of MEDI-507 to prevent, treat or ameliorate one or more symptoms associated with an autoimmune disorder or inflammatory disorder. Preferably, such doses are administered intravaneously to a subject with an autoimmune disorder or an inflammatory disorder.

In a specific embodiment, a subject is administered one or more unit doses of 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 mg to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.25 mg to 2.5 mg, 1 mg, to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg of MEDI-507 to prevent, treat or ameliorate one or more symptoms associated with an autoimmune disorder or inflammatory disorder. In another embodiment, a subject is administered one or more unit doses of 0.1 mg, 0.25 mg, 0.5 mg, 1 mg, 1.5 mg, 2 mg, 3 mg, 4 mg, 5 mg, 6 mg, 7 mg, 8 mg, 9 mg, 10 mg, 11 mg, 12 mg, 13 mg, 14 mg, 15 mg, or 16 mg of MEDI-507 to prevent, treat or ameliorate one or more symptoms associated with an autoimmune disorder or inflammatory disorder. Preferably, the unit doses of MEDI-507 are administered subcutaneously to a subject with an autoimmune or inflammatory disorder.

In another embodiment, a subject is administered one or more doses of a prophylactically or therapeutically effective amount of MEDI-507, wherein the prophylactically or therapeutically effective amount is not the same for each dose. In another embodiment, a subject, preferably a human, is administered one or more doses of a prophylactically or therapeutically effective amount of MEDI-507, wherein the dose of a prophylactically or therapeutically effective amount MEDI-507 administered to said subject is increased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 μg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In another embodiment, a subject, preferably a human, is administered one or more doses of a prophylactically or therapeutically effective amount of MEDI-507, wherein the dose of a prophylactically or therapeutically effective amount of MEDI-507 administered to said subject is decreased by, e.g., 0.01 μg/kg, 0.02 μg/kg, 0.04 μg/kg, 0.05 μg/kg, 0.06 μg/kg, 0.08 μg/kg, 0.1 μg/kg, 0.2 μg/kg, 0.25 μg/kg, 0.5 μg/kg, 0.75 μg/kg, 1 tg/kg, 1.5 μg/kg, 2 μg/kg, 4 μg/kg, 5 μg/kg, 10 μg/kg, 15 μg/kg, 20 μg/kg, 25 μg/kg, 30 μg/kg, 35 μg/kg, 40 μg/kg, 45 μg/kg, 50 μg/kg, 55 μg/kg, 60 μg/kg, 65 μg/kg, 70 μg/kg, 75 μg/kg, 80 μg/kg, 85 μg/kg, 90 μg/kg, 95 μg/kg, 100 μg/kg, or 125 μg/kg, as treatment progresses.

In yet another embodiment, a subject is administered one or more doses of a prophylactically or therapeutically effective amount of one or more immunomodulatory agents, wherein the dose of a prophylactically or therapeutically effective amount of said agent(s) administered to said subject achieves in said subject a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³. In another embodiment, a subject is administered a dose of a prophylactically or therapeutically effective amount of one of more CD2 binding molecule, wherein administration of the dose to said subject achieves a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³. In a preferred embodiment, a subject is administered a dose of a prophylactically or therapeutically effective amount of MEDI-507, wherein administration of the dose of MEDI-507 to said subject achieves in said subject a mean absolute lymphocyte count of approximately 500 cells/mm³ to below 1500 cells/mm³, preferably below 1400 cells/mm³, below 1300 cells/mm³, below 1250 cells/mm³, below 1200 cells/mm³, below 1100 cells/mm³ or below 1000 cell/mm³.

In other embodiments, a subject is administered one or more doses of a prophylactically or therapeutically effective amount of one or more CD2 binding molecules, wherein the dose of a prophylactically or therapeutically effective amount of said CD2 binding molecules administered achieves at least 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, up to at least 80% of CD2 polypeptide being bound by CD2 binding molecules. In yet other embodiments, a subject is administered one or more doses of a prophylactically or therapeutically effective amount of MEDI-507, wherein the dose of a prophylactically or therapeutically effective amount of MEDI-507 administered achieves at least 20% to 25%, 25% to 30%, 30% to 35%, 35% to 40%, 40% to 45%, 45% to 50%, 50% to 55%, 55% to 60%, 60% to 65%, 65% to 70%, 70% to 75%, 75% to 80%, up to at least 80% of CD2 polypeptide being bound by CD2 binding molecules.

5.4.1 Gene Therapy

In a specific embodiment, nucleic acids comprising sequences encoding one or more prophylactic or therapeutic agents, are administered to treat, prevent or ameliorate one or more symptoms associated with an inflammatory or autoimmune disease, by way of gene therapy. Gene therapy refers to therapy performed by the administration to a subject of an expressed or expressible nucleic acid. In this embodiment of the invention, the nucleic acids produce their encoded prophylactic or therapeutic agent that mediates a prophylactic or therapeutic effect.

Any of the methods for gene therapy available in the art can be used according to the present invention. Exemplary methods are described below.

For general reviews of the methods of gene therapy, see Goldspiel et al., 1993, Clinical Pharmacy 12:488-505; Wu and Wu, 1991, Biotherapy 3:87-95; Tolstoshev, 1993, Ann. Rev. Pharmacol. Toxicol. 32:573-596; Mulligan, Science 260:926-932 (1993); and Morgan and Anderson, 1993, Ann. Rev. Biochem. 62:191-217; May, 1993, TIBTECH 11(5):155-215. Methods commonly known in the art of recombinant DNA technology which can be used are described in Ausubel et al. (eds.), Current Protocols in Molecular Biology, John Wiley & Sons, NY (1993); and Kriegler, Gene Transfer and Expression, A Laboratory Manual, Stockton Press, NY (1990).

In a preferred aspect, a composition of the invention comprises nucleic acids encoding a prophylactic or therapeutic agent, said nucleic acids being part of an expression vector that expresses the prophylactic or therapeutic agent in a suitable host. In particular, such nucleic acids have promoters, preferably heterologous promoters, operably linked to the antibody coding region, said promoter being inducible or constitutive, and, optionally, tissue-specific. In another particular embodiment, nucleic acid molecules are used in which the prophylactic or therapeutic agent coding sequences and any other desired sequences are flanked by regions that promote homologous recombination at a desired site in the genome, thus providing for intrachromosomal expression of the antibody encoding nucleic acids (Koller and Smithies, 1989, Proc. Natl. Acad. Sci. USA 86:8932-8935; Zijlstra et al., 1989, Nature 342:435-438). In certain embodiments, the prophylactic or therapeutic agent expressed. In other embodiments the prophylactic or therapeutic agent expressed is an agent known to be useful for, or has been or is currently being used in the prevention, treatment or amelioration of one or more symptoms associated with an inflammatory or autoimmune disease. In a preferred embodiment, the prophylactic or therapeutic agent expressed is VITAXIN™.

Delivery of the nucleic acids into a subject may be either direct, in which case the subject is directly exposed to the nucleic acid or nucleic acid-carrying vectors, or indirect, in which case, cells are first transformed with the nucleic acids in vitro, then transplanted into the subject. These two approaches are known, respectively, as in vivo or ex vivo gene therapy.

In a specific embodiment, the nucleic acid sequences are directly administered in vivo, where it is expressed to produce the encoded product. This can be accomplished by any of numerous methods known in the art, e.g., by constructing them as part of an appropriate nucleic acid expression vector and administering it so that they become intracellular, e.g., by infection using defective or attenuated retrovirals or other viral vectors (see U.S. Pat. No. 4,980,286), or by direct injection of naked DNA, or by use of microparticle bombardment (e.g., a gene gun; Biolistic, Dupont), or by a matrix with in situ scaffolding in which the nucleic acid sequence is contained (see, e.g., European Patent No. EP 0 741 785 Bi and U.S. Pat. No. 5,962,427), or coating with lipids or cell-surface receptors or transfecting agents, encapsulation in liposomes, microparticles, or microcapsules, or by administering them in linkage to a peptide which is known to enter the nucleus, by administering it in linkage to a ligand subject to receptor-mediated endocytosis (see, e.g., Wu and Wu, 1987, J. Biol. Chem. 262:4429-4432) (which can be used to target cell types specifically expressing the receptors), etc. In another embodiment, nucleic acid-ligand complexes can be formed in which the ligand comprises a fusogenic viral peptide to disrupt endosomes, allowing the nucleic acid to avoid lysosomal degradation. In yet another embodiment, the nucleic acid can be targeted in vivo for cell specific uptake and expression, by targeting a specific receptor (see, e.g., PCT Publications WO 92/06180; WO 92/22635;

W092/203 16; W093/14188, WO 93/20221). Alternatively, the nucleic acid can be introduced intracellularly and incorporated within host cell DNA for expression, by homologous recombination (Koller and Smithies, 1989, Proc. Natl. Acad. Sci. USA 86:8932-8935; and Zijlstra et al., 1989, Nature 342:435-438).

In a specific embodiment, viral vectors that contains nucleic acid sequences encoding a prophylactic or therapeutic agent are used. For example, a retroviral vector can be used (see Miller et al., 1993, Meth. Enzymol. 217:581-599). These retroviral vectors contain the components necessary for the correct packaging of the viral genome and integration into the host cell DNA. The nucleic acid sequences encoding the antibody to be used in gene therapy are cloned into one or more vectors, which facilitates delivery of the gene into a subject. More detail about retroviral vectors can be found in Boesen et al., 1994, Biotherapy 6:291-302, which describes the use of a retroviral vector to deliver the mdr 1 gene to hematopoietic stem cells in order to make the stem cells more resistant to chemotherapy. Other references illustrating the use of retroviral vectors in gene therapy are: Clowes et al., 1994, J. Clin. Invest. 93:644-651; Klein et al., 1994, Blood 83:1467-1473; Salmons and Gunzberg, 1993, Human Gene Therapy 4:129-141; and Grossman and Wilson, 1993, Curr. Opin. in Genetics and Devel. 3:110-114.

Adenoviruses are other viral vectors that can be used in gene therapy. Adenoviruses are especially attractive vehicles for delivering genes to respiratory epithelia. Adenoviruses naturally infect respiratory epithelia where they cause a mild disease. Other targets for adenovirus-based delivery systems are liver, the central nervous system, endothelial cells, and muscle. Adenoviruses have the advantage of being capable of infecting non-dividing cells. Kozarsky and Wilson, 1993, Current Opinion in Genetics and Development 3:499-503 present a review of adenovirus-based gene therapy. Bout et al., 1994, Human Gene Therapy 5:3-10 demonstrated the use of adenovirus vectors to transfer genes to the respiratory epithelia of rhesus monkeys. Other instances of the use of adenoviruses in gene therapy can be found in Rosenfeld et al., 1991, Science 252:431-434; Rosenfeld et al., 1992, Cell 68:143-155; Mastrangeli et al., 1993, J. Clin. Invest. 91:225-234; PCT Publication W094/12649; and Wang et al., 1995, Gene Therapy 2:775-783. In a preferred embodiment, adenovirus vectors are used.

Adeno-associated virus (AAV) has also been proposed for use in gene therapy (Walsh et al., 1993, Proc. Soc. Exp. Biol. Med. 204:289-300; and U.S. Pat. No. 5,436,146).

Another approach to gene therapy involves transferring a gene to cells in tissue culture by such methods as electroporation, lipofection, calcium phosphate mediated transfection, or viral infection. Usually, the method of transfer includes the transfer of a selectable marker to the cells. The cells are then placed under selection to isolate those cells that have taken up and are expressing the transferred gene. Those cells are then delivered to a subject.

In this embodiment, the nucleic acid is introduced into a cell prior to administration in vivo of the resulting recombinant cell. Such introduction can be carried out by any method known in the art, including but not limited to transfection, electroporation, microinjection, infection with a viral or bacteriophage vector containing the nucleic acid sequences, cell fusion, chromosome-mediated gene transfer, microcellmediated gene transfer, spheroplast fusion, etc. Numerous techniques are known in the art for the introduction of foreign genes into cells (see, e.g., Loeffler and Behr, 1993, Meth. Enzymol. 217:599-618; Cohen et al., 1993, Meth. Enzymol. 217:618-644; Clin. Pharma. Ther. 29:69-92 (1985)) and may be used in accordance with the present invention, provided that the necessary developmental and physiological functions of the recipient cells are not disrupted. The technique should provide for the stable transfer of the nucleic acid to the cell, so that the nucleic acid is expressible by the cell and preferably heritable and expressible by its cell progeny.

The resulting recombinant cells can be delivered to a subject by various methods known in the art. Recombinant blood cells (e.g., hematopoietic stem or progenitor cells) are preferably administered intravenously. The amount of cells envisioned for use depends on the desired effect, patient state, etc., and can be determined by one skilled in the art.

Cells into which a nucleic acid can be introduced for purposes of gene therapy encompass any desired, available cell type, and include but are not limited to epithelial cells, endothelial cells, keratinocytes, fibroblasts, muscle cells, hepatocytes; blood cells such as T lymphocytes, B lymphocytes, natural killer (NK) cells, monocytes, macrophages, neutrophils, eosinophils, megakaryocytes, granulocytes; various stem or progenitor cells, in particular hematopoietic stem or progenitor cells, e.g., as obtained from bone marrow, umbilical cord blood, peripheral blood, fetal liver, etc.

In a preferred embodiment, the cell used for gene therapy is autologous to the subject.

In an embodiment in which recombinant cells are used in gene therapy, nucleic acid sequences encoding a prophylactic or therapeutic agent are introduced into the cells such that they are expressible by the cells or their progeny, and the recombinant cells are then administered in vivo for prophylactic or therapeutic effect. In a specific embodiment, stem or progenitor cells are used. Any stem and/or progenitor cells which can be isolated and maintained in vitro can potentially be used in accordance with this embodiment of the present invention (see e.g., PCT Publication WO 94/08598; Stemple and Anderson, 1992, Cell 7 1:973-985; Rheinwald, 1980, Meth. Cell Bio. 21A:229; and Pittelkow and Scott, 1986, Mayo Clinic Proc. 61:771).

In a specific embodiment, the nucleic acid to be introduced for purposes of gene therapy comprises a constitutive, tissue-specific, or inducible promoter operably linked to the coding region. In a preferred embodiment, the nucleic acid to be introduced for purposes of gene therapy comprises an inducible promoter operably linked to the coding region, such that expression of the nucleic acid is controllable by controlling the presence or absence of the appropriate inducer of transcription.

10 5.5. Characterization and Demonstration of Prophylactic or Therapeutic Utility of Combination Therapy

Several aspects of the pharmaceutical compositions or prophylactic or therapeutic agents of the invention are preferably tested in vitro, in a cell culture system, and in an animal model organism, such as a rodent animal model system, for the desired therapeutic activity prior to use in humans. For example, assays which can be used to determine whether administration of a specific pharmaceutical composition is indicated, include cell culture assays in which a patient tissue sample is grown in culture, and exposed to or otherwise contacted with a pharmaceutical composition, and the effect of such composition upon the tissue sample is observed. The tissue sample can be obtained by biopsy from the patient. This test allows the identification of the therapeutically most effective prophylactic or therapeutic molecule(s) for each individual patient. In various specific embodiments, in vitro assays can be carried out with representative cells of cell types involved in an autoimmune or inflammatory disorder (e.g., T cells), to determine if a pharmaceutical composition of the invention has a desired effect upon such cell types.

Combinations of prophylactic and/or therapeutic agents can be tested in suitable animal model systems prior to use in humans. Such animal model systems include, but are not limited to, rats, mice, chicken, cows, monkeys, pigs, dogs, rabbits, etc. Any animal system well-known in the art may be used. In a specific embodiment of the invention, combinations of prophylactic and/or therapeutic agents are tested in a mouse model system. Such model systems are widely used and well-known to the skilled artisan. Prophylactic and/or therapeutic agents can be administered repeatedly. Several aspects of the procedure may vary. Said aspects include the temporal regime of administering the prophylactic and/or therapeutic agents, and whether such agents are administered separately or as an admixture.

The anti-inflammatory activity of the combination therapies of invention can be determined by using various experimental animal models of inflammatory arthritis known in the art and described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993). Experimental and spontaneous animal models of inflammatory arthritis and autoimmune rheumatic diseases can also be used to assess the anti-inflammatory activity of the combination therapies of invention. The following are some assays provided as examples and not by limitation.

The principle animal models for arthritis or inflammatory disease known in the art and widely used include: adjuvant-induced arthritis rat models, collagen-induced arthritis rat and mouse models and antigen-induced arthritis rat, rabbit and hamster models, all described in Crofford L. J. and Wilder R. L., “Arthritis and Autoimmunity in Animals”, in Arthritis and Allied Conditions: A Textbook of Rheumatology, McCarty et al.(eds.), Chapter 30 (Lee and Febiger, 1993), incorporated herein by reference in its entirety.

The anti-inflammatory activity of the combination therapies of invention can be assessed using a carrageenan-induced arthritis rat model. Carrageenan-induced arthritis has also been used in rabbit, dog and pig in studies of chronic arthritis or inflammation. Quantitative histomorphometric assessment is used to determine therapeutic efficacy. The methods for using such a carrageenan-induced arthritis model is described in Hansra P. et al., “Carrageenan-Induced Arthritis in the Rat,” Inflammation, 24(2): 141-155, (2000). Also commonly used are zymosan-induced inflammation animal models as known and described in the art.

The anti-inflammatory activity of the combination therapies of invention can also be assessed by measuring the inhibition of carrageenan-induced paw edema in the rat, using a modification of the method described in Winter C. A. et al., “Carrageenan-Induced Edema in Hind Paw of the Rat as an Assay for Anti-inflammatory Drugs” Proc. Soc. Exp. Biol Med. 111, 544-547, (1962). This assay has been used as a primary in vivo screen for the anti-inflammatory activity of most NSAIDs, and is considered predictive of human efficacy. The anti-inflammatory activity of the test prophylactic or therapeutic agents is expressed as the percent inhibition of the increase in hind paw weight of the test group relative to the vehicle dosed control group.

In a specific embodiment of the invention where the experimental animal model used is adjuvant-induced arthritis rat model, body weight can be measured relative to a control group to determine the anti-inflammatory activity of the combination therapies of invention. Combination therapies tested may include, but are not limited to, combinations comprising any integrin α_{V}β₃ antagonist functionally homologous to VITAXIN™, a TNF-α inhibitor, and a chemotherapeutic agent. RENBREL™, the rat homolog of ENBREL™, which functions as a TNF-α inhibitor, may also be tested in combination therapies in rat models.

Alternatively, the efficacy of the combination therapies of the invention can be assessed using assays that determine bone loss. Animal models such as ovariectomy-induced bone resorption mice, rat and rabbit models are known in the art for obtaining dynamic parameters for bone formation. Using methods such as those described by Yositake et al. or Yamamoto et al., bone volume is measured in vivo by microcomputed tomography analysis and bone histomorphometry analysis. Yoshitake et al., “Osteopontin-Deficient Mice Are Resistant to Ovariectomy-Induced Bone Resorption,” Proc. Natl. Acad. Sci. 96:8156-8160, (1999); Yamamoto et al., “The Integrin Ligand Echistatin Prevents Bone Loss in Ovariectomized Mice and Rats,” Endocrinology 139(3):1411-1419, (1998), both incorporated herein by reference in their entirety.

Additionally, animal models for inflammatory bowel disease can also be used to assess the efficacy of the combination therapies of invention (Kim eta 1., 1992, Scand. J. Gastroentrol. 27:529-537; Strober, 1985, Dig. Dis. Sci. 30(12 Suppl):3S-10S). Ulcerative cholitis and Crohn's disease are human inflammatory bowel diseases that can be induced in animals. Sulfated polysaccharides including, but not limited to amylopectin, carrageen, amylopectin sulfate, and dextran sulfate or chemical irritants including but not limited to trinitrobenzenesulphonic acid (TNBS) and acetic acid can be administered to animals orally to induce inflammatory bowel diseases.

Animal models for asthma can also be used to assess the efficacy of the combination therapies of invention. An example of one such model is the murine adoptive transfer model in which aeroallergen provocation of TH1 or TH2 recipient mice results in TH effector cell migration to the airways and is associated with an intense neutrophilic (TH1) and eosinophilic (TH2) lung mucosal inflammatory response (Cohn et al., 1997, J. Exp.

Med. 1861737-1747).

Animal models for autoimmune disorders can also be used to assess the efficacy of the combination therapies of invention. Animal models for autoimmune disorders such as type 1 diabetes, thyroid autoimmunity, sytemic lupus eruthematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Further, any assays known to those skilled in the art can be used to evaluate the prophylactic and/or therapeutic utility of the combinatorial therapies disclosed herein for autoimmune and/or inflammatory diseases.

The effect of the combination therapies of the invention on peripheral blood lymphocyte counts can be monitored/assessed using standard techniques known to one of skill in the art. Peripheral blood lymphocytes counts in a subject can be determined by, e.g., obtaining a sample of peripheral blood from said subject, separating the lymphocytes from other components of peripheral blood such as plasma using, e.g., Ficoll-Hypaque (Pharmacia) gradient centrifugation, and counting the lymphocytes using trypan blue. Peripheral blood T-cell counts in subject can be determined by, e.g., separating the lymphocytes from other components of peripheral blood such as plasma using, e.g., a use of Ficoll-Hypaque (Pharmacia) gradient centrifugation, labeling the T-cells with an antibody directed to a T-cell antigen such as CD3, CD4, and CD8 which is conjugated to FITC or phycoerythrin, and measuring the number of T-cells by FACS.

The percentage of CD2 polypeptides expressed by peripheral blood T-cells bound by CD2 binding molecules prior or after, or both prior to and after the administration of one or more doses of CD2 binding molecules and/or one or more doses of one or more other prophylactic or therapeutic agents can be assessed using standard techniques known to one of skill in the art. The percentage of CD2 polypeptides expressed by peripheral blood T-cells bound by CD2 binding molecules can be determined by, e.g., obtaining a sample of peripheral blood from a subject, separating the lymphocytes from other components of peripheral blood such as plasma using, e.g., Ficoll-Hypaque (Pharmacia) gradient centrifugation, and labeling the T-cells with an anti-CD2 binding molecule antibody conjugated to FITC and an antibody directed to a T-cell antigen such as CD3, CD4 or CD4 which is conjugated to phycoerythrin, and determining the number of T-cells labeled with anti-CD2 binding molecule antibody relative to the number of T-cells labeled with an antibody directed to a T-cell antigen using FACS.

The toxicity and/or efficacy of the prophylactic and/or therapeutic protocols of the instant invention can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index and it can be expressed as the ratio LD₅₀/ED₅₀. Prophylactic and/or therapeutic agents that exhibit large therapeutic indices are preferred. While prophylactic and/or therapeutic agents that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. The dosage of such agents lies preferably within a range of circulating concentrations that include the ED₅₀ with little or no toxicity. The dosage may vary within this range depending upon the dosage form employed and the route of administration utilized. For any agent used in the method of the invention, the therapeutically effective dose can be estimated initially from cell culture assays. A dose may be formulated in animal models to achieve a circulating plasma concentration range that includes the IC₅₀ (i e., the concentration of the test compound that achieves a half-maximal inhibition of symptoms) as determined in cell culture. Such information can be used to more accurately determine useful doses in humans. Levels in plasma may be measured, for example, by high performance liquid chromatography.

5.6 Methods of Producing Antibodies

The antibodies that immunospecifically bind to an antigen can be produced by any method known in the art for the synthesis of antibodies, in particular, by chemical synthesis or preferably, by recombinant expression techniques.

Polyclonal antibodies immunospecific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a Don-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

Accordingly, the present invention provides methods of generating monoclonal antibodies as well as antibodies produced by the method comprising culturing a hybridoma cell secreting an antibody of the invention wherein, preferably, the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; PCT application No. PCT/GB91/O1 134; PCT publication Nos. WO 90/02809, WO 91/10737, WO 92/01047, WO 92/18619, WO 93/1 1236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743 and 5,969,108; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. Preferably, the vectors for expressing the VH or VL domains comprise an EF-lα promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, e.g., IgG, using techniques known to those of skill in the art.

For some uses, including in vivo use of antibodies in humans and in vitro detection assays, it may be preferable to use human or chimeric antibodies. Completely human antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and PCT publications WO 98/46645, WO 98/50433, WO 98/24893, W098/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., PCT publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules such as antibodies having a variable region derived from a human antibody and a non-human immunoglobulin constant region. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, and 4,8 16397, which are incorporated herein by reference in their entirety. Chimeric antibodies comprising one or more CDRs from human species and framework regions from a non-human immunoglobulin molecule can be produced using a variety of techniques known in the art including, for example, CDR-grafting (EP 239,400; PCT publication No. WO 91/09967; and U.S. Pat. Nos. 5,225,539, 5,530,101, and 5,585,089), veneering or resurfacing (EP 592,106; EP 519,596; Padlan, 1991, Molecular Immunology 28(4/5):489-498; Studnicka et al., 1994, Protein Engineering 7(6):805-814; and Roguska et al., 1994, PNAS 91:969-973), and chain shuffling (U.S. Pat. No. 5,565,332). In a preferred embodiment, chimeric antibodies comprise a human CDR3 having an amino acid sequence of any one of the CDR3 listed in Table 1 or Table 2and non-human framework regions. Often, framework residues in the framework regions will be substituted with the corresponding residue from the CDR donor antibody to alter, preferably improve, antigen binding. These framework substitutions are identified by methods well known in the art, e.g., by modeling of the interactions of the CDR and framework residues to identify framework residues important for antigen binding and sequence comparison to identify unusual framework residues at particular positions. (See, e.g., Queen et al., U.S. Pat. No. 5,585,089; and Riechmann et al., 1988, Nature 332:323, which are incorporated herein by reference in their entireties.)

Further, the antibodies that immunospecifically bind to an antigen (e.g., CD2 polypeptide) can, in turn, be utilized to generate anti-idiotype antibodies that “mimic” an antigen using techniques well known to those skilled in the art. (See, e.g, Greenspan & Bona, 1989, FASEB J. 7(5):437-444; and Nissinoff, 1991, J. Immunol. 147(8):2429-2438

5.6.1 Polynucleotide Sequences Encoding an Antibody

The invention provides polynucleotides comprising a nucleotide sequence encoding an antibody or fragment thereof that immunospecifically binds to an antigen. The invention also encompasses polynucleotides that hybridize under high stringency, intermediate or lower stringency hybridization conditions, e.g., as defined supra, to polynucleotides that encode an antibody of the invention.

The polynucleotides may be obtained, and the nucleotide sequence of the polynucleotides determined, by any method known in the art. The nucleotide sequence of antibodies immunospecific for a desired antigen can be obtained, e.g., from the literature or a database such as GenBank. Since the amino acid sequences of VITAXIN™ is known, nucleotide sequences encoding this antibody can be determined using methods well known in the art, i.e., nucleotide codons known to encode particular amino acids are assembled in such a way to generate a nucleic acid that encodes the antibody. Such a polynucleotide encoding the antibody may be assembled from chemically synthesized oligonucleotides (e.g., as described in Kutmeier et al., 1994, BioTechniques 17:242), which, briefly, involves the synthesis of overlapping oligonucleotides containing portions of the sequence encoding the antibody, annealing and ligating of those oligonucleotides, and then amplification of the ligated oligonucleotides by PCR.

Alternatively, a polynucleotide encoding an antibody may be generated from nucleic acid from a suitable source. If a clone containing a nucleic acid encoding a particular antibody is not available, but the sequence of the antibody molecule is known, a nucleic acid encoding the immunoglobulin may be chemically synthesized or obtained from a suitable source (e.g., an antibody cDNA library, or a cDNA library generated from, or nucleic acid, preferably poly A+RNA, isolated from, any tissue or cells expressing the antibody, such as hybridoma cells selected to express an antibody of the invention) by PCR amplification using synthetic primers hybridizable to the 3′ and 5′ ends of the sequence or by cloning using an oligonucleotide probe specific for the particular gene sequence to identify, e.g., a cDNA clone from a cDNA library that encodes the antibody. Amplified nucleic acids generated by PCR may then be cloned into replicable cloning vectors using any method well known in the art.

Once the nucleotide sequence of the antibody is determined, the nucleotide sequence of the antibody may be manipulated using methods well known in the art for the manipulation of nucleotide sequences, e.g., recombinant DNA techniques, site directed mutagenesis, PCR, etc. (see, for example, the techniques described in Sambrook et al., 1990, Molecular Cloning, A Laboratory Manual, 2d Ed., Cold Spring Harbor Laboratory, Cold Spring Harbor, N.Y. and Ausubel et al., eds., 1998, Current Protocols in Molecular Biology, John Wiley & Sons, NY, which are both incorporated by reference herein in their entireties), to generate antibodies having a different amino acid sequence, for example to create amino acid substitutions, deletions, and/or insertions.

In a specific embodiment, one or more of the CDRs is inserted within framework regions using routine recombinant DNA techniques. The framework regions may be naturally occurring or consensus framework regions, and preferably human framework regions (see, e.g., Chothia et al., 1998, J. Mol. Biol. 278: 457-479 for a listing of human framework regions). Preferably, the polynucleotide generated by the combination of the framework regions and CDRs encodes an antibody that specifically binds to a particular antigen. Preferably, as discussed supra, one or more amino acid substitutions may be made within the framework regions, and, preferably, the amino acid substitutions improve binding of the antibody to its antigen. Additionally, such methods may be used to make amino acid substitutions or deletions of one or more variable region cysteine residues participating in an intrachain disulfide bond to generate antibody molecules lacking one or more intrachain disulfide bonds. Other alterations to the polynucleotide are encompassed by the present invention and within the skill of the art.

5.6.2 Recombinant Expression of an Antibody

Recombinant expression of an antibody that immunospecifically binds to an antigen requires construction of an expression vector containing a polynucleotide that encodes the antibody. Once a polynucleotide encoding an antibody molecule of the invention has been obtained, the vector for the production of the antibody molecule may be produced by recombinant DNA technology using techniques well-known in the art. See, e.g., U.S. Pat. No. 6,331,415, which is incorporated herein by reference in its entirety. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody or a portion thereof, or a heavy or light chain CDR, operably linked to a promoter. Such vectors may include the nucleotide sequence encoding the constant region of the antibody molecule (see, e.g., PCT Publication WO 86/05807; PCT Publication WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or portion thereof, or a single chain antibody of the invention, operably linked to a heterologous promoter. In preferred embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (e.g. E. coli and B. subtilis) transformed with recombinant bacteriophage DNA, plasmid DNA or cosmid DNA expression vectors containing antibody coding sequences; yeast (e.g, Saccharomyces Pichia) transformed with recombinant yeast expression vectors containing antibody coding sequences; insect cell systems infected with recombinant virus expression vectors (e.g., baculovirus) containing antibody coding sequences; plant cell systems infected with recombinant virus expression vectors (e.g., cauliflower mosaic virus, CaMV; tobacco mosaic virus, TMV) or transformed with recombinant plasmid expression vectors (e.g., Ti plasmid) containing antibody coding sequences; or mammalian cell systems (e.g., COS, CHO, BHK, 293, NS0, and 3T3 cells) harboring recombinant expression constructs containing promoters derived from the genome of mammalian cells (e.g., metallothionein promoter) or from mammalian viruses (e.g., the adenovirus late promoter; the vaccinia virus 7.5K promoter). Preferably, bacterial cells such as Escherichia coli, and more preferably, eukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies which immunospecifically bind to one or more antigens is regulated by a constitutive promoter, inducible promoter or tissue specific promoter.

In bacterial systems, a number of expression vectors may be advantageously selected depending upon the use intended for the antibody molecule being expressed. For example, when a large quantity of such a protein is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (e.g., glycosylation) and processing (e.g., cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NS0 (a murine myeloma cell line that does not endogenously produce any immunoglobulin chains), CRL7O3O and HsS78Bst cells.

For long-term, high-yield production of recombinant proteins, stable expression is preferred. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-2 15); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et aL (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol.3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

5.7. Methods of Producing Polypeptides and Fusion Proteins

Polypeptides and fusion proteins can be produced by standard recombinant DNA techniques or by protein synthetic techniques, e.g., by use of a peptide synthesizer. For example, a nucleic acid molecule encoding a polypeptide or a fusion protein can be synthesized by conventional techniques including automated DNA synthesizers. Alternatively, PCR amplification of gene fragments can be carried out using anchor primers which give rise to complementary overhangs between two consecutive gene fragments which can subsequently be annealed and reamplified to generate a chimeric gene sequence (see, e.g., Current Protocols in Molecular Biology, Ausubel et al., eds., John Wiley & Sons, 1992). Moreover, a nucleic acid encoding a bioactive molecule can be cloned into an expression vector containing the Fc domain or a fragment thereof such that the bioactive molecule is linked in-frame to the Fc domain or Fc domain fragment.

Methods for fusing or conjugating polypeptides to the constant regions of antibodies are known in the art. See, e.g., U.S. Pat. Nos. 5,336,603, 5,622,929, 5,359,046, 5,349,053, 5,447,851, 5,723,125, 5,783,181, 5,908,626, 5,844,095, and 5,112,946; EP 307,434; EP 367,166; EP 394,827; PCT publications WO 91/06570, WO 96/04388, WO 96/22024, WO 97/34631, and WO 99/04813; Ashkenazi et al., 1991, Proc. Natl. Acad. Sci. USA 88: 10535-10539; Traunecker et al., 1988, Nature, 331:84-86; Zheng et al., 1995, J. Immunol. 154:5590-5600; and Vil et al., 1992, Proc. Natl. Acad. Sci. USA 89:11337-11341, which are incorporated herein by reference in their entireties.

The nucleotide sequences encoding a bioactive molecule and an Fc domain or fragment thereof may be an be obtained from any information available to those of skill in the art (i.e., from Genbank, the literature, or by routine cloning). The nucleotide sequence coding for a polypeptide a fusion protein can be inserted into an appropriate expression vector, i.e., a vector which contains the necessary elements for the transcription and translation of the inserted protein-coding sequence. A variety of host-vector systems may be utilized in the present invention to express the protein-coding sequence. These include but are not limited to mammalian cell systems infected with virus (e.g., vaccinia virus, adenovirus, etc.); insect cell systems infected with virus (e.g., baculovirus); microorganisms such as yeast containing yeast vectors; or bacteria transformed with bacteriophage, DNA, plasmid DNA, or cosmid DNA. The expression elements of vectors vary in their strengths and specificities. Depending on the host-vector system utilized, any one of a number of suitable transcription and translation elements may be used.

The expression of a polypeptide or a fusion protein may be controlled by any promoter or enhancer element known in the art. Promoters which may be used to control the expression of the gene encoding fusion protein include, but are not limited to, the SV40 early promoter region (Bernoist and Chambon, 1981, Nature 290:304-310), the promoter contained in the 3′ long terminal repeat of Rous sarcoma virus (Yamamoto, et al., 1980, Cell 22:787-797), the herpes thymidine kinase promoter (Wagner et al., 1981, Proc. Natl. Acad. Sci. U.S.A. 78:1441-1445), the regulatory sequences of the metallothionein gene (Brinster et al., 1982, Nature 296:39-42), the tetracycline (Tet) promoter (Gossen et al., 1995, Proc. Nat. Acad. Sci. USA 89:5547-5551); prokaryotic expression vectors such as the β-lactamase promoter (Villa-Kamaroff, et al., 1978, Proc. Natl. Acad. Sci. U.S.A. 75:3727-3731), or the tac promoter (DeBoer, et al., 1983, Proc. Natl. Acad. Sci. U.S.A. 80:21-25; see also “Useful proteins from recombinant bacteria” in Scientific American, 1980, 242:74-94); plant expression vectors comprising the nopaline synthetase promoter region (Herrera-Estrella et al., Nature 303:209-213) or the cauliflower mosaic virus 35S RNA promoter (Gardner, et al., 1981, Nucl. Acids Res. 9:287 1), and the promoter of the photosynthetic enzyme ribulose biphosphate carboxylase (Herrera-Estrella et al., 1984, Nature 310:115-120); promoter elements from yeast or other fungi such as the Gal 4 promoter, the ADC (alcohol dehydrogenase) promoter, PGK (phosphoglycerol kinase) promoter, alkaline phosphatase promoter, and the following animal transcriptional control regions, which exhibit tissue specificity and have been utilized in transgenic animals: elastase I gene control region which is active in pancreatic acinar cells (Swift et al., 1984, Cell 38:639-646; Ornitz et al., 1986, Cold Spring Harbor Symp. Quant. Biol. 50:399-409; MacDonald, 1987, Hepatology 7:425-515); insulin gene control region which is active in pancreatic beta cells (Hanahan, 1985, Nature 315:115-122), immunoglobulin gene control region which is active in lymphoid cells (Grosschedl et al., 1984, Cell 38:647-658; Adames et al., 1985, Nature 318:533-538; Alexander et al., 1987, Mol. Cell. Biol. 7:1436-1444), mouse mammary tumor virus control region which is active in testicular, breast, lymphoid and mast cells (Leder et al., 1986, Cell 45:485-495), albumin gene control region which is active in liver (Pinkert et al., 1987, Genes and Devel. 1:268-276), alpha-fetoprotein gene control region which is active in liver (Krumlauf et al., 1985, Mol. Cell. Biol. 5:1639-1648; Hammer et al., 1987, Science 235:53-58; alpha 1-antitrypsin gene control region which is active in the liver (Kelsey et al., 1987, Genes and Devel. 1:161-171), beta-globin gene control region which is active in myeloid cells (Mogram et al., 1985, Nature 315:338-340; Kollias et al., 1986, Cell 46:89-94; myelin basic protein gene control region which is active in oligodendrocyte cells in the brain (Readhead et al., 1987, Cell 48:703-712); myosin light chain-2 gene control region which is active in skeletal muscle (Sani, 1985, Nature 314:283-286); neuronal-specific enolase (NSE) which is active in neuronal cells (Morelli et al., 1999, Gen. Virol. 80:571-83); brain-derived neurotrophic factor (BDNF) gene control region which is active in neuronal cells (Tabuchi et al., 1998, Biochem. Biophysic. Res. Corn. 253:818-823); glial fibrillary acidic protein (GFAP) promoter which is active in astrocytes (Gomes et al., 1999, Braz J Med Biol Res 32(5):619-631; Morelli et al., 1999, Gen. Virol. 80:571-83) and gonadotropic releasing hormone gene control region which is active in the hypothalamus (Mason et al., 1986, Science 234:1372-1378).

In a specific embodiment, the expression of a polypeptide or a fusion protein is regulated by a constitutive promoter. In another embodiment, the expression of a polypeptide or a fusion protein is regulated by an inducible promoter. In another embodiment, the expression of a polypeptide or a fusion protein is regulated by a tissue-specific promoter.

In a specific embodiment, a vector is used that comprises a promoter operably linked to a polypeptide- or a fusion protein-encoding nucleic acid, one or more origins of replication, and, optionally, one or more selectable markers (e.g., an antibiotic resistance gene).

In mammalian host cells, a number of viral-based expression systems may be utilized. In cases where an adenovirus is used as an expression vector, the polypeptide or fusion protein coding sequence may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 81:355-359). Specific initiation signals may also be required for efficient translation of inserted fusion protein coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see Bittner et al., 1987, Methods in Enzymol. 153:51-544).

Expression vectors containing inserts of a gene encoding a polypeptide or a fusion protein can be identified by three general approaches: (a) nucleic acid hybridization, (b) presence or absence of “marker” gene functions, and (c) expression of inserted sequences. In the first approach, the presence of a gene encoding a polypeptide or a fusion protein in an expression vector can be detected by nucleic acid hybridization using probes comprising sequences that are homologous to an inserted gene encoding the polypeptide or the fusion protein, respectively. In the second approach, the recombinant vector/host system can be identified and selected based upon the presence or absence of certain “marker” gene functions (e.g., thymidine kinase activity, resistance to antibiotics, transformation phenotype, occlusion body formation in baculovirus, etc.) caused by the insertion of a nucleotide sequence encoding a polypeptide or a fusion protein in the vector. For example, if the nucleotide sequence encoding the fusion protein is inserted within the marker gene sequence of the vector, recombinants containing the gene encoding the fusion protein insert can be identified by the absence of the marker gene function. In the third approach, recombinant expression vectors can be identified by assaying the gene product (e.g., fusion protein) expressed by the recombinant. Such assays can be based, for example, on the physical or functional properties of the fusion protein in in vitro assay systems, e.g., binding with anti-bioactive molecule antibody.

In addition, a host cell strain may be chosen which modulates the expression of the inserted sequences, or modifies and processes the gene product in the specific fashion desired. Expression from certain promoters can be elevated in the presence of certain inducers; thus, expression of the genetically engineered fusion protein may be controlled. Furthermore, different host cells have characteristic and specific mechanisms for the translational and post-translational processing and modification (e.g., glycosylation, phosphorylation of proteins). Appropriate cell lines or host systems can be chosen to ensure the desired modification and processing of the foreign protein expressed. For example, expression in a bacterial system will produce an unglycosylated product and expression in yeast will produce a glycosylated product. Eukaryotic host cells which possess the cellular machinery for proper processing of the primary transcript, glycosylation, and phosphorylation of the gene product may be used. Such mammalian host cells include, but are not limited to, CHO, VERY, BHK, Hela, COS, MDCK, 293, 3T3, W138, NSO, and in particular, neuronal cell lines such as, for example, SK-N-AS, SK-N-FI, SK-N-DZ human neuroblastomas (Sugimoto et al., 1984, J. Natl. Cancer Inst. 73: 51-57), SK-N-SH human neuroblastoma (Biochim. Biophys. Acta, 1982, 704: 450-460), Daoy human cerebellar medulloblastoma (He et al., 1992, Cancer Res. 52: 1144-1148) DBTRG-05MG glioblastoma cells (Kruse et al., 1992, In Vitro Cell. Dev. Biol. 28A: 609-614), IMR-32 human neuroblastoma (Cancer Res., 1970, 30: 2110-2118), 1321N1 human astrocytoma (Proc. Natl Acad. Sci. USA ,1977, 74: 4816), MOG-G-CCM human astrocytoma (Br. J. Cancer, 1984, 49: 269), U87MG human glioblastoma-astrocytoma (Acta Pathol. Microbiol. Scand., 1968, 74: 465-486), A172 human glioblastoma (Olopade et al., 1992, Cancer Res. 52: 2523-2529), C6 rat glioma cells (Benda et al., 1968, Science 161: 370-371), Neuro-2a mouse neuroblastoma (Proc. Natl. Acad. Sci. USA, 1970, 65: 129-136), NB41A3 mouse neuroblastoma (Proc. Natl. Acad. Sci. USA, 1962, 48: 1184-1190), SCP sheep choroid plexus (Bolin et al., 1994, J. Virol. Methods 48: 211-221), G355-5, PG-4 Cat normal astrocyte (Haapala et al., 1985, J. Virol. 53: 827-833), Mpf ferret brain (Trowbridge et al., 1982, In Vitro 18: 952-960), and normal cell lines such as, for example, CTX TNA2 rat normal cortex brain (Radany et al., 1992, Proc. Natl. Acad. Sci. USA 89: 6467-6471) such as, for example, CRL7030 and Hs578Bst. Furthermore, different vector/host expression systems may effect processing reactions to different extents.

For long-term, high-yield production of recombinant proteins, stable expression is preferred. For example, cell lines which stably express a polypeptide or a fusion protein may be engineered. Rather than using expression vectors which contain viral origins of replication, host cells can be transformed with DNA controlled by appropriate expression control elements (e.g., promoter, enhancer, sequences, transcription termina-tors, polyadenylation sites, etc.), and a selectable marker. Following the introduction of the foreign DNA, engineered cells may be allowed to grow for 1-2 days in an enriched medium, and then are switched to a selective medium. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express a polypeptide or a fusion protein that immunospecifically binds to a CD2 polypeptide. Such engineered cell lines may be particularly useful in screening and evaluation of compounds that affect the activity of a polypeptide or a fusion protein that immunospecifically binds to a CD2 polypeptide.

A number of selection systems may be used, including but not limited to the herpes simplex virus thymidine kinase (Wigler, et al., 1977, Cell 11:223), hypoxanthine-guanine phosphoribosyltransferase (Szybalska & Szybalski, 1962, Proc. Natl. Acad. Sci. USA 48:2026), and adenine phosphoribosyltransferase (Lowy, et al., 1980, Cell 22:817) genes can be employed in tk-, hgprt- or aprt-cells, respectively. Also, antimetabolite resistance can be used as the basis of selection for dhfr, which confers resistance to methotrexate (Wigler, et al., 1980, Natl. Acad. Sci. USA 77:3567; O'Hare, et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Colberre-Garapin, et al., 1981, J. Mol. Biol. 150:1); and hygro, which confers resistance to hygromycin (Santerre, et al., 1984, Gene 30:147) genes.

Once a polypeptide or a fusion protein of the invention has been produced by recombinant expression, it may be purified by any method known in the art for purification of a protein, for example, by chromatography (e.g., ion exchange, affinity, particularly by affinity for the specific antigen after Protein A, and sizing column chromatography), centrifugation, differential solubility, or by any other standard technique for the purification of proteins.

5.8. Articles of Manufacture

The present invention also encompasses a finished packaged and labeled pharmaceutical product. The present invention provides article of manufactures comprising packaging material and a pharmaceutical composition of the invention in suitable form for administration to a subject contained within said packaging material. In particular, the present invention provides article of manufactures comprising packaging material and a pharmaceutical composition of the invention in suitable form for administration to a subject contained within said packaging material wherein said pharmaceutical composition comprises one or more integrin α_{V}β₃ antagonists, one or more prophylactic or therapeutic agents other than integrin α_{V}β₃ antagonists, and a pharmaceutically acceptable carrier.

In a specific embodiment, an article of manufacture comprises packaging material and a pharmaceutical composition in suitable form for administration to a subject contained within said packaging material, wherein said pharmaceutical composition comprises an integrin α_{V}β₃ antagonist, an anti-inflammatory agent, and a pharmaceutically acceptable carrier. In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, an immunomodulatory agent, and a pharmaceutically acceptable carrier.

In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, a CD2 binding molecule, and a pharmaceutically acceptable carrier. In a preferred embodiment, an article of manufacture comprises packaging material and a pharmaceutical composition in suitable form for administration to a human, preferably a human with an autoimmune or inflammatory disorder, contained within said packaging material, wherein said pharmaceutical composition comprises VITAXIN™ antagonist, MEDI-507, and a pharmaceutically acceptable carrier.

In another embodiment, an article of manufacture comprises packaging material and a pharmaceutical composition in suitable form for administration to a subject, preferably a human, and most preferably a human with an autoimmune or inflammatory disorder, contained within said packaging material, wherein said pharmaceutical composition comprises an integrin α_{V}β₃ antagonist, a TNF-α antagonist, and a pharmaceutically acceptable carrier. In a preferred embodiment, an article of manufacture comprises packaging material and a pharmaceutical composition in suitable form for administration to a human, preferably a human with an autoimmune or inflammatory disorder, contained within said packaging material, wherein said pharmaceutical composition comprises an integrin α_{V}β₃ antagonist, a ENBREL™ or REMICADE™, and a pharmaceutically acceptable carrier.

As with any pharmaceutical product, the packaging material and container of the articles of manufacture of the invention are designed to protect the stability of the product during storage and shipment. More specifically, the invention provides an article of manufacture comprising packaging material, such as a box, bottle, tube, vial, container, sprayer, insufflator, intravenous (i.v.) bag, envelope and the like; and at least one unit dosage form of a pharmaceutical agent contained within said packaging material. The invention also provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. The invention further provides an article of manufacture comprising packaging material, such as a box, bottle, tube, vial, container, sprayer, insufflator, intravenous (i.v.) bag, envelope and the like; and at least one unit dosage form of each pharmaceutical agent contained within said packaging material. This article of manufacture includes the appropriate unit dosage form in an appropriate vessel or container such as a glass vial or other container that is hermetically sealed. In the case of dosage forms suitable for parenteral administration the active ingredient is sterile and suitable for administration as a particulate free solution. In other words, the invention encompasses both parenteral solutions and lyophilized powders, each being sterile, and the latter being suitable for reconstitution prior to injection. Alternatively, the unit dosage form may be a solid suitable for oral, transdermal, topical or mucosal delivery. In a preferred embodiment, the unit dosage form is suitable for intravenous, intramuscular or subcutaneous delivery. Thus, the invention encompasses solutions, preferably sterile, suitable for each delivery route.

The articles of manufacture of the invention may include instructions regarding the use or administration of a pharmaceutical composition, or other informational material that advises the physician, technician or patient on how to appropriately prevent or treat the disease or disorder in question. In other words, the article of manufacture includes instruction means indicating or suggesting a dosing regimen including, but not limited to, actual doses, monitoring procedures, total lymphocyte and T-cell counts and other monitoring information. The present invention provides that the adverse effects that may be reduced or avoided by the methods of the invention are indicated in informational material enclosed in an article of manufacture for use in preventing, treating or ameliorating one or more symptoms associated with an inflammatory or autoimmune disorder. Adverse effects that may be reduced or avoided by the methods of the invention include but are not limited to vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation. Since some of the prophylactic or therapeutic agents used in the accordance with the invention may be immunosuppressive, prolonged immunosuppression may increase the risk of infection, including opportunistic infections. Prolonged and sustained immunosuppression may also result in an increased risk of developing certain types of cancer.

Further, the information material enclosed in an article of manufacture for use in preventing, treating or ameliorating one or more symptoms with an autoimmune or inflammatory disorder can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

The information material can also indicate that cytokine release syndrome is an acute clinical syndrome, temporally associated with the administration of certain activating anti-T cell antibodies. Cytokine release syndrome has been attributed to the release of cytokines by activated lymphocytes or monocytes. The clinical manifestations for cytokine release syndrome have ranged from a more frequently reported mild, self-limited, “flu-like” illness to a less frequently reported severe, life-threatening, shock-like reaction, which may include serious cardiovascular, pulmonary and central nervous system manifestations. The syndrome typically begins approximately 30 to 60 minutes after administration (but may occur later) and may persist for several hours. The frequency and severity of this symptom complex is usually greatest with the first dose. With each successive dose, both the incidence and severity of the syndrome tend to diminish. Increasing the amount of a dose or resuming treatment after a hiatus may result in a reappearance of the syndrome. As mentioned above, the invention encompasses methods of treatment and prevention that avoid or reduce one or more of the adverse effects discussed herein.

The following example is presented by way of illustration and not by way of limitation of the scope of the invention.

### 6. EXAMPLE

### Treatment of Patients with Rheumatoid Arthritis

A phase I, open label, dose escalation study is designed to assess pharmacokinetics and safety of VITAXIN™ in patients with active rheumatoid arthritis. Rheumatoid arthritis that is active is defined as the presence of at least 2 swollen joints involving the hands, wrists, knees or ankles. Rheumatoid arthritis patients currently receive therapy with methotrexate with or without additional anti-rheumatic agents such as etanercept, infliximab, sulfasalazine, or hydroxychloroquine. Patients currently receiving treatment with stable doses of nonsteroidal anti-inflammatory drugs or prednisone (<I0 mg/day) are permitted to continue these medications. Patients currently receiving therapy with cyclosporin A, leflunomide, or gold salts discontinue these drugs at least 4 weeks before beginning VITAXIN™ administration.

Patients are administered a single IV dose and then, beginning 4 weeks later, are analyzed following administration of repeated weekly IV doses at the same dose over a period of 12 weeks. VITAXIN™ safety and potential changes in disease activity over 26 weeks of IV dosing is also be assessed. Different groups of patients are treated and evaluated similarly but receive doses of 1 mg/kg, 2 mg/kg, 4 mg/kg, or 8 mg/kg.

VITAXIN™ is formulated at 5 mg/ml and 10 mg/ml for IV injection. A formulation of 80 mg/ml is required for repeated subcutaneous administration.

Changes in disease activity are assessed through tender and swollen joint counts, patient and physician global scores for pain and disease activity, and the ESR/CRP. Progression of structural joint damage are assessed by quantitative scoring of X-rays of hands, wrists, and feet (Sharp method). Changes in functional status are evaluated using the Health Assessment Questionnaire (HAQ), and quality of life changes are assessed with the SF-36.

VITAXIN™ can be prepared and formulated in accordance with the disclosure of U.S. Ser. No. 09/339,922, filed Jun. 24, 1999 which is herein incorporated by reference in its entirety.

The present invention is not to be limited in scope by the exemplified embodiments, which are intended as illustrations of single aspects of the invention. Indeed, various modifications of the invention in addition to those shown and described herein will become apparent to those skilled in the art from the foregoing description. Such modifications are intended to fall within the scope of the appended claims.

All patents, patent applications and non-patent publications cited herein are incorporated by reference in their entirety to the same extent as if each individual patent, patent application or non-patent publication was specifically and individually indicated to be incorporated herein by reference.

What is claimed is:

1. A method of treating or ameliorating an inflammatory disorder or an autoimmune disorder or one or more symptoms thereof, said method comprising administering to a subject in need thereof a prophylactically or therapeutically effective amount of one or more integrin α_{V}β₃ antagonists and a prophylactically or therapeutically effective amount of one or more immunomodulatory agents.

2. The method of claim 1 wherein said α_{V}β₃ antagonist is VITAXIN™ or an antigen-binding fragment thereof.

3. A method of treating or ameliorating an inflammatory disorder or an autoimmune disorder or one or more symptoms thereof, said method comprising administering to a subject in need thereof a prophylactically or therapeutically effective amount of one or more integrin α_{V}β₃ antagonists and a prophylactically or therapeutically effective amount of one or more anti-inflammatory agents.

4. The method of claim 3 wherein said α_{V}β₃ antagonist is VITAXIN™ or an antigen-binding fragment thereof.

5. A method of treating or ameliorating an inflammatory disorder or an autoimmune disorder or one or more symptoms thereof, said method comprising administering to a subject in need thereof a prophylactically or therapeutically effective amount of one or more integrin α_{V}β₃ antagonists and a prophylactically or therapeutically effective amount of one or more TNF-α antagonists.

6. The method of claim 5 wherein said α_{V}β₃ antagonist is VITAXIN™ or an antigen-binding fragment thereof.

7. A method of treating or ameliorating an inflammatory disorder or an autoimmune disorder or one or more symptoms thereof, said method comprising administering to a subject in need thereof a prophylactically or therapeutically effective amount of one or more integrin α_{V}β₃ antagonists and a prophylactically or therapeutically effective amount of one or more CD2 binding molecules.

8. The method of claim 7 wherein said α_{V}β₃ antagonist is VITAXIN™ or an antigen-binding fragment thereof.

9. The method of claim 1 or 2, wherein at least one immunomodulatory agent is a small organic molecule.

10. The method of claim 1 or 2, wherein at least one immunomodulatory agent is a T cell receptor modulator or a cytokine receptor modulator.

11. The method of claim 9, wherein the small organic molecule is methotrexate, leflunomide, cyclophosphamide, cyclosporine A, FK506, mycophenolate mofetil, rapamycin, mizoribine, deoxyspergualin, brequinar, a malononitriloamide, a steroid or a corticosteriod.

12. The method of claim 10, wherein the T cell receptor modulator is an antibody, peptide or a fusion protein which immunospecifically binds to a T cell receptor.

13. The method of claim 12, wherein the antibody that immunospecifically binds to a T cell receptor is a monoclonal antibody or an antigen-binding fragment thereof.

14. The method of claim 13, wherein the monoclonal antibody is a human or humanized monoclonal antibody.

15. The method of claim 13, wherein the monoclonal antibody is an anti-CD2 monoclonal antibody, an anti-CD4 monoclonal antibody, an anti-CD8 monoclonal antibody or an anti-CD40 monoclonal antibody.

16. The method of claim 12, wherein the fusion protein is CTLA4-Ig.

17. The method of claim 10, wherein the cytokine receptor modulator is a cytokine, a fragment of a cytokine, a fusion protein or an antibody that immunospecifically binds to a cytokine receptor.

18. The method of claim 10, wherein the cytokine receptor modulator is a peptide, polypeptide, fusion protein or an antibody that inmmunospecifically binds to a cytokine.

19. The method of claim 17, wherein the antibody that immunospecifically binds to a cytokine receptor is a monoclonal antibody or an antigen-binding fragment thereof.

20. The method of claim 19, wherein the monoclonal antibody is a human or humanized monoclonal antibody.

21. The method of claim 17, wherein the antibody is an anti-IL-2 receptor antibody and anti-IL-12 receptor antibodies.

22. The method of claim 18, wherein the antibody that immunospecifically binds to a cytokine is a monoclonal antibody or an antigen-binding fragment thereof.

23. The method of claim 22, wherein the monoclonal antibody is a human or humanized monoclonal antibody.

24. The method of claim 18, wherein the antibody is anti-TNF-α antibody, an anti-IL-1β antibody, or an anti-IL-6 antibody.

25. The method of claim 17, wherein the cytokine is IL-4 or IL-10.

26. The method of claim 18, wherein the polypeptide is a fragment of a cytokine receptor that immunospecifically binds to a cytokine.

27. The method of claim 26, wherein the fragment is a portion of the extracellular domain of a TNF-α receptor.

28. The method of claim 3 or 4, wherein at least one anti-inflammatory agent is a non-steroidal anti-inflammatory drug.

29. The method of claim 27, wherein the non-steriodal anti-inflammatory drug is aspirin, ibuprofen, diclofenac, nabumetone, naproxen, or ketoproten.

30. The method of claim 5 or 6, wherein the TNF-α antagonist is ENBREL™ or REMICADE™.

31. The method of claim 5 or 6 further comprising administering to said subject a prophylactically or therapeutically effective amount of methotrexate.

32. The method of claim 7 or 8, wherein the CD2 binding molecule is a peptide, polypeptide, fusion protein or an antibody that immunospecifically binds to a CD2 polypeptide.

33. The method of claim 32, wherein the fusion protein is LFA-3TIP.

34. The method of claim 7 or 8 further comprising administering to said subject a prophylactically or therapeutically effective amount of a non-steriodal anti-inflammatory drug.

35. The method of claim 34, wherein the non-steriodal anti-inflammatory drug is aspirin, ibuprofen, diclofenac, nabumetone, naproxen, or ketoproten.

36. The method of claim 7 or 8 further comprising administering to said subject a prophylactically or therapeutically effective amount of one or more immunomodulatory agents other than a CD2 binding molecule.

37. A method of treating or ameliorating an inflammatory disorder or an autoimmune disorder or one or more symptoms thereof, said method comprising administering to a subject in need thereof a prophylactically or therapeutically effective amount of one or more integrin α_{V}β₃ antagonists and a prophylactically or therapeutically effective amount of MEDI-507 or an antigen-binding fragment thereof.

38. A method of treating or ameliorating an inflammatory disorder or an autoimmune disorder or one or more symptoms thereof, said method comprising administering to a subject in need thereof a prophylactically or therapeutically effective amount of VITAXIN™ or an antigen-binding fragment thereof and a prophylactically or therapeutically effective amount of MEDI-507 or an antigen-binding fragment thereof.

39. The method of claim 37 or 38 further comprising administering to said subject a prophylactically or therapeutically effective amount of one or more TNF-α antagonists.

40. The method of claim 37 or 38 further comprising administering to said subject a prophylactically or therapeutically effective amount of methotrexate.

41. The method of claim 37 or 38 further comprising administering to said subject a prophylactically or therapeutically effective amount of one or more TNF-α antagonists and a prophylactically or therapeutically effective amount of methotrexate.

42. The method of claim 39, wherein at least one TNF-α antagonist is ENBREL™ or REMICADE™.

43. The method of claim 1, 3, 5, 7 or 37, wherein at least one integrin α_{V}β₃ antagonist is an anti-integrin α_{V}β₃ antibody.

44. The method of claim 44, wherein the anti-α_{V}β₃ antibody is a monoclonal antibody or an antigen-binding fragment thereof.

45. The method of claim 45, wherein the monoclonal antibody is a human or humanized monoclonal antibody.

46. The method of claim 1, 2, 3, 4, 5, 6, 7, 8, 37 or 38, wherein the inflammatory disorder is asthma, encephilitis, inflammatory bowel disease, chronic obstructive pulmonary disease (COPD), arthritis, or an allergic disorder.

47. The method of claim 1, 2, 3, 4, 7, 8, 37 or 38, wherein the autoimmune disorder is rheumatoid arthritis, psoriatic arthritis, ankylosing spondylitis, Reiter's Syndrome, inflammatory bowel disease associated arthritis, an undifferentitated spondyloarthropathy, psoriasis, or an undifferentiated arthropathy.

48. The method of claim 1, 2, 3, 4, 5, 6, 7, 8, 37 or 38, wherein the subject is a human.

49. The method of claim 5, 6, 7, 8, 37 or 38, wherein the subject is a human who is or has previously been treated with one or more TNF-α antagonists.

50. The method of claim 5, 6, 7, 8, 37 or 38, wherein the subject is a human who is not currently being treated with a TNF-α antagonist or methotrexate.

51. The method of claim 5, 6, 7, 8, 37 or 38, wherein the subject is a human with an inflammatory disorder that is refractory to treatment with a TNF-α antagonist, a non-steriodal anti-inflammatory agent or methotrexate alone.

52. The method of claim 2, 4, 6, 8 or 38, wherein VITAXIN™ or an antigen-binding fragment thereof is administered orally, topically, intravenously, intramuscularly or subcutaneously to said subject.

53. The method of claim 37 or 38, wherein MEDI-507 or an antigen-binding fragment thereof is administered orally, topically, intravenously, intramuscularly or subcutaneously to said subject.

54. The method of claim 1, 3, 5, 7 or 37, wherein said integrin α_{V}β₃ antagonists are not small organic molecules.

55. The method of claim 1, 3, 5, 7 or 37, wherein at least one integrin α_{V}β₃ antagonist is a small organic molecule.

56. The method of claim 7 or 8, wherein said CD2 binding molecules are not small organic molecules.

57. The method of claim 7 or 8, wherein at least one CD2 binding molecule is a small organic molecule.

58. A method of treating or ameliorating an inflammatory disorder or an autoimmune disorder or one or more symptoms thereof, said method comprising administering to a subject in need thereof a prophylactically or therapeutically effective amount of VITAXIN™ or an antigen-binding fragment thereof, a prophylactically or therapeutically effective amount of REMICADE™ or ENBREL™, and a prophylactically or therapeutically effective amount of methotrexate.

59. The method of claim 58, wherein the amount of VITAXIN™ or an antigen-binding fragment thereof administered to said subject is a dosage of about 0.1 mg/kg to 10 mg/kg.

60. The method of claim 58, wherein the amount of REMICADE™ administered to said subject is a dosage of about 0.1 mg/kg to 10 mg/kg.

61. The method of claim 58, wherein the amount of ENBREL™ administered to said subject is a dosage of about 0.01 mg/kg to 10 mg/kg.

62. The method of claim 58, wherein the methotrexate administered to said subject is a dosage of about 0.01 mg/kg to 3 mg/kg.

63. A pharmaceutical composition comprising an integrin α_{V}β₃ antagonist, a TNF-α antagonist, and a pharmaceutically acceptable carrier.

64. A pharmaceutical composition comprising an integrin α_{V}β₃ antagonist, a CD2 binding molecule, and a pharmaceutically acceptable carrier.

65. The composition of claim 63 further comprising methotrexate.

66. The composition of claim 63 or 65, wherein the integrin α_{V}β₃ antagonist is VITAXIN™ or an antigen-binding fragment thereof.

67. The composition of claim 63, wherein the TNF-α antagonist is REMICADE™ or ENBREL™.

68. The composition of claim 64, wherein the CD2 binding molecule is LFA3TIP, MEDI-507, or antigen-binding fragment of MEDI-507.

69. A pharmaceutical composition comprising VITAXIN™ or an antigen-binding fragment thereof, MEDI-507 or an antigen-binding fragment thereof, and a pharmaceutically acceptable carrier.

70. An article of manufacture comprising packaging material and a pharmaceutical composition in suitable form for administration to a human contained within said packaging material, wherein said pharmaceutical composition comprises VITAXIN™ or an antigen-binding fragment thereof, MEDI-507 or an antigen-binding fragment thereof, a pharmaceutically acceptable carrier, and instructions contained with said packaging material which suggests a dosing regimen for the prevention or treatment of an inflammatory disorder or an autoimmune disorder.
